(19) United States
(12) Patent Application Publication
(10) Pub. No.: US 2006/0069086 A1
(43) Pub. Date: Mar. 30, 2006

(54) Methods for regulating neurotransmitter systems by inducing counteradaptations

(76) Inventor: Alexander Michalow, Bourbonnais, IL (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/234,850

(22) Filed: Sep. 23, 2005

Related U.S. Application Data
(60) Provisional application No. 60/612,155, filed on Sep. 23, 2004.

(51) Int. Cl. A61K 31/551; A61K 31/405; A61K 31/343; A61K 31/137

(52) U.S. Cl. 514/220; 514/469; 514/649; 514/419

(57) ABSTRACT

The present invention relates to methods for regulating neurotransmitter systems by inducing a counteradaptation response. According to one embodiment of the invention, a method for regulating a neurotransmitter includes the step of repeatedly administering a ligand for a receptor in the neurotransmitter system, with a ratio of administration half-life to period between administrations of no greater than 1/2. The methods of the present invention may be used to address a whole host of undesirable mental and neurological conditions.

### CROSS-REFERENCE TO RELATED APPLICATIONS

This application claims the benefit under 35 U.S.C. § 119(e) of U.S. Provisional Patent Application Ser. No. 60/612,155 entitled “COUNTER-ADAPTATION THERAPY FOR TREATMENT OF DEPRESSION AND OTHER MENTAL CONDITIONS,” and filed on Sep. 23, 2004. The above-referenced provisional application is hereby incorporated herein by reference in its entirety.

### BACKGROUND OF THE INVENTION

1. Field of the Invention

The present invention relates generally to neurotransmitter systems associated with undesirable mental and neurological conditions. The present invention relates more particularly to methods for regulating these neurotransmitter systems by inducing counteradaptative responses.

2. Technical Background

Mood, mood disorders and related conditions are a result of a complex web of central nervous system events that interrelate many neurotransmitter systems. A most common mood disorder is depression. Depression is a clinical diagnosis with numerous somatic and mental symptoms, which is due to an alteration of numerous neurotransmitter systems. While the neurotransmitter systems most commonly related with depression are the norepinephrine and serotonin systems, current research indicates that other systems, such as the substance P system, the dynorphin system (kappa receptors), and the endogenous endorphin system (mu and delta opiate receptors) are also involved in depression. Further, these neurotransmitter systems are also related to a whole host of other undesirable mental and neurological conditions, including bipolar disorders, obsessive-compulsive disorders, anxiety, phobias, stress disorders, substance abuse, sexual disorders, eating disorders, motivational disorders and pain disorders.

Conventional strategies for treating neurotransmitter-linked conditions are centered on improving abnormally high or low levels of synaptic neurotransmitters. Conventional therapeutic agents work to directly regulate the functioning of the neurotransmitter systems. Such agents may be anxiolytic agents, hypnotic agents, or selective reuptake inhibitors, and include benzodiazepines (e.g., diazepam, lorazepam, alprazolam, temazepam, flurazepam, and chlodiazepoxide), TCAs, MAOIs, SSRIs (e.g., fluoxetine hydrochloride), NRIs, SNRIs, CRF modulating agents, serotonin pre-synaptic autoreceptor antagonists, 5HT₁ agonist, GABA-A modulating agents, serotonin 5H_{2C} and/or 5H_{2B} modulating agents, beta-3 adrenoceptor agonists, NMDA antagonists, V1B antagonists, GPCR modulating agents, dynorphin antagonists, and substance P antagonists.

Conventional therapeutic agents and methods, while somewhat effective, suffer from a few disadvantages. For example, use of many conventional therapeutic agents is attended by side effects, such as sexual dysfunction, nausea nervousness, fatigue, dry mouth, blurred vision and weight gain. Further, patients can adapt or build up a resistance to conventional therapeutic agents with repeated use, making them lose efficacy over time.

### SUMMARY OF THE INVENTION

One embodiment of the present invention relates to a method of regulating a neurotransmitter system by inducing a counteradaptation in a patient, the neurotransmitter system including a type of receptor linked to an undesirable mental or neurological condition. The method comprises the step of: repeatedly administering to the patient a ligand for the type of receptor, each administration having an administration half-life, thereby causing the ligand to bind receptors of that type during a first time period associated with each administration, thereby inducing a counteradaptation, wherein the counteradaptation causes the regulation of the neurotransmitter system, and wherein the ratio of the administration half-life to the period between administrations is no greater than 1/2.

In another embodiment of the invention, a method is provided for inducing a regulation of a neurotransmitter system in a patient, the neurotransmitter system including a type of receptor linked to an undesirable mental or neurological condition. The method comprising the steps of: inducing a counteradaptation by giving the patient a ligand for the type of receptor; then repeatedly administering to the patient a ligand for the type of receptor, each administration having an administration half-life, thereby causing the ligand to bind receptors of that type during a first time period associated with each administration, thereby maintaining or improving the counteradaptation, wherein the counteradaptation causes the regulation of the neurotransmitter system, and wherein the ratio of the administration half-life to the period between administrations is no greater than 1/2.

In one aspect of the invention, the neurotransmitter system is the SP system; the type of receptor is SP receptors; the ligand is an SP receptor agonist; the undesirable mental or neurological condition is positively linked to the receptors; and the counteradaption causes a down-regulation of the SP system.

In another aspect of the invention, the neurotransmitter system is the endogenous endorphin system; the type of receptor is mu and/or delta opiate receptors; the ligand is a mu and/or delta opiate receptor agonist; the undesirable mental or neurological condition is negatively linked to the receptors; and the counteradaption causes an up-regulation of the endogenous endorphin system.

In yet another aspect of the invention, the neurotransmitter system is the dynorphin system; the type of receptor is kappa receptors; the ligand is a kappa receptor agonist; the undesirable mental or neurological condition is positively linked to the receptors; and the counteradaption causes a down-regulation of the dynorphin system.

In still yet another aspect of the invention, the neurotransmitter system is the serotonin system; and the counteradaption causes an up-regulation of the serotonin system. Thus, in one embodiment of this aspect of the invention, the type of receptor is serotonin pre-synaptic autoreceptors; the ligand is a serotonin pre-synaptic autoreceptor agonist; and the undesirable mental or neurological condition is positively linked to the receptors. In another embodiment of this aspect of the invention the type of receptor is serotonin post-synaptic receptors; the ligand is a serotonin post-synaptic autoreceptor antagonist; and the undesirable mental or neurological condition is negatively linked to the serotonin post-synaptic autoreceptors.

In still yet another aspect of the invention, the neurotransmitter system is the norepinephrine system; and the counteradaption causes an up-regulation of the norepinephrine system. Thus, in one embodiment of this aspect of the invention, the type of receptor is norepinephrine pre-synaptic alpha-2 adrenergic receptors; the ligand is a norepinephrine pre-synaptic alpha-2 adrenergic receptor agonist; and the undesirable mental or neurological condition is positively linked to the receptors. In another embodiment of this aspect of the invention the type of receptor is norepinephrine post-synaptic adrenergic receptors; the ligand is a norepinephrine post-synaptic adrenergic receptor antagonist; and the undesirable mental or neurological condition is negatively linked to the norepinephrine post-synaptic adrenergic receptors.

In yet another embodiment of the invention, a method is provided for inducing a regulation of a neurotransmitter system, the neurotransmitter system including a type of receptors linked to an undesirable mental or neurological condition. The method comprising the step of: repeatedly administering to the patient a ligand for the type of receptor, each administration having an administration half-life, thereby causing the ligand to bind a substantial fraction of receptors of that type during a first time period associated with each administration, thereby inducing a counteradaptation; wherein the counteradaptation causes the regulation of the neurotransmitter system during a second time period associated with each administration, the second time period being subsequent to the first time period.

The methods of the present invention result in a number of advantages over prior art methods. For example, the methods of the present invention can be used to address a whole host of undesirable mental and neurological conditions with reduced side effects. In certain embodiments of the invention, the desired therapeutic benefit can be timed to coincide with a desired time of day or task to be performed by the patient.

Additional features and advantages of the invention will be set forth in the detailed description which follows, and in part will be readily apparent to those skilled in the art from the description or recognized by practicing the invention as described in the written description and claims hereof, as well as in the appended drawings.

It is to be understood that both the foregoing general description and the following detailed description are merely exemplary of the invention, and are intended to provide an overview or framework for understanding the nature and character of the invention as it is claimed.

The accompanying drawings are included to provide a further understanding of the invention, and are incorporated in and constitute a part of this specification. The drawings are not necessarily to scale, and sizes of various elements may be distorted for clarity. The drawings illustrate one or more embodiment(s) of the invention, and together with the description serve to explain the principles and operation of the invention.

### BRIEF DESCRIPTION OF THE DRAWINGS

FIG. 1 is a graph of in vivo ligand concentration (part a) and mood vs. time (part b) according to one embodiment of the invention;

FIG. 2 is a graph of mood vs. time for several administrations of a ligand according to another embodiment of the invention;

FIG. 3 is a graph of in vivo ligand concentration vs. time for the administration via a single injection of a ligand with a relatively long compound half-life;

FIG. 4 is a graph of in vivo ligand concentration vs. time for the administration via time-release transdermal patch of a ligand with a relatively short compound half-life;

FIG. 5 is a graph of in vivo ligand concentration vs. time for the administration via time-release transdermal patch of a ligand with a relatively short compound half-life, when the patch is removed during the administration; and

FIG. 6 is a graph of in vivo ligand concentration (part a) and mood vs. time (part b) according to another embodiment of the invention.

### DETAILED DESCRIPTION OF THE INVENTION

The present invention relates generally to the regulation of neurotransmitter systems by exploiting the patient's response to a pharmaceutical agent (a “counteradaptation”), rather than by relying on the direct effect of the agent for an improved clinical effect. In general, pharmaceutical agents are chosen so that the counteradaptation is beneficial to the patient and eventually provides the desired long-term effect. The methods of the present invention differ from conventional methods in that the direct effect of the agent is a modulation of neurotransmitter receptors that is generally associated with a worsening of symptoms. In response to the direct effect of the agent, however, the brain responds by a counteradaptation, resulting in the desired regulation of the neurotransmitter system when any direct effect of the agent wears off. The regulation may be any change in neurotransmitter functioning, and may be, for example, an up-regulation or a down-regulation. A specific acute response is induced directly in order to generate a desired long-term effect indirectly. In a simple analogy, just as euphoria-stimulating agents such as morphine and cocaine result in depression upon their withdrawal, dysphoria-stimulating agents result in “anti-depression” upon their withdrawal.

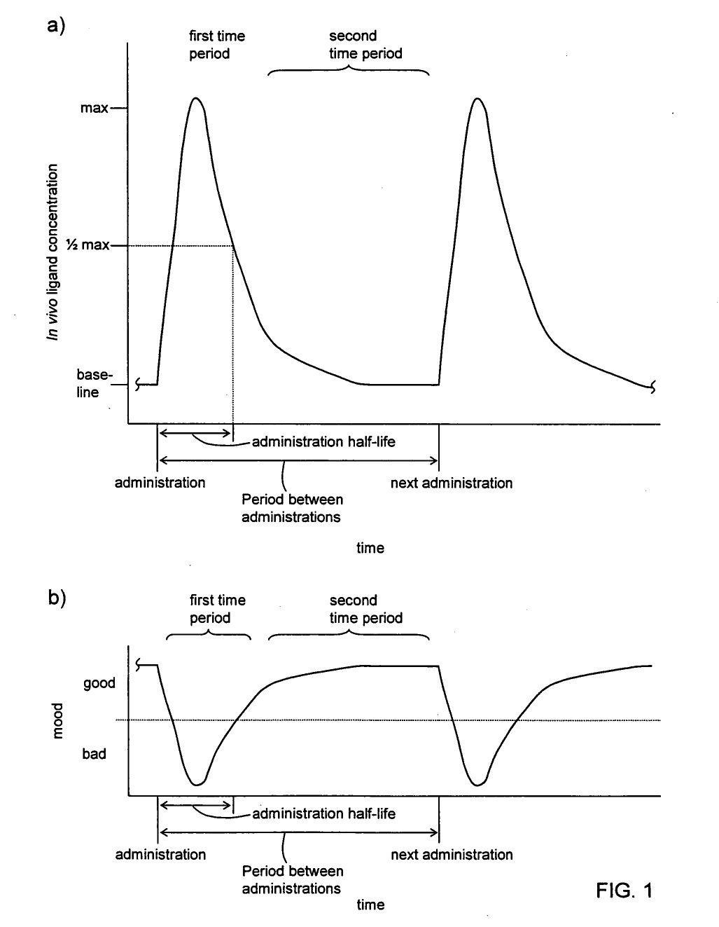

One embodiment of the present invention relates to a method of inducing a regulation of a neurotransmitter system. Generally, a neurotransmitter system is a system of natural neurotransmitter compounds and synaptic receptors that participates in central nervous system signal transmission. The neurotransmitter system includes a type of receptors linked to an undesirable mental or neurological condition. FIG. 1 includes a graph of in vivo ligand concentration versus time for a method according to one embodiment of the invention. As illustrated in FIG. 1, the method includes the step of repeatedly administering to a patient a ligand for the type of receptor, thereby causing the ligand to bind receptors of that type during a first time period associated with each administration. As used herein, a ligand is a compound that binds to (e.g., interacts with in either a covalent or non-covalent fashion) receptors of the type of receptor, and may be, for example, an agonist for the receptor or an antagonist for the receptor. The binding of the ligand to the receptors induces a counteradaptation, which causes the regulation of the neurotransmitter system. FIG. 1 shows a couple of administrations of ligand occurring in the middle of the method, and not the first couple of administrations. Each administration is a single cycle in which the in vivo concentration of the ligand begins at a baseline level, goes up to a maximum level, and drops back down to the baseline level. The graph of FIG. 1 shows two such administrations. Depending on the dosing regimen, each administration of the ligand may be performed, for example, by giving the patient a single unit dose (e.g., pill, capsule) or injection; multiple unit doses or injections; or continuously (e.g., intravenous or slow-release patch).

Examples of types of neurotransmitter systems and types of receptors with which the method may be practiced include the Substance P system, in which the type of receptors may be NK-1, NK-2 and/or NK-3 receptors; the endogenous endorphin system in which the type of receptors may be mu and/or delta opiate receptors; the dynorphin system in which the type of receptors may be kappa receptors; the serotonin system in which the type of receptors may be inhibitory serotonin pre-synaptic autoreceptors (e.g., 5HT_{1A} and/or 5HT_{1B} autoreceptors) and/or serotonin post-synaptic receptors (e.g. 5HT₁, 5HT₂, 5HT₃, 5HT₄, 5HT₅, 5HT₆ and/or 5HT₇ receptors); and the norepinephrine system in which the type of receptors may be inhibitory norepinephrine pre-synaptic alpha-2 adrenergic receptors and/or norepinephrine post-synaptic adrenergic receptors. These neurotransmitter systems and receptor types are linked to various undesirable mental and neurological conditions, as would be appreciated by the skilled artisan.

The undesirable mental or neurological condition is linked to a type of receptor in the neurotransmitter system. If the undesirable mental or neurological condition is exacerbated by the binding of the receptor to its natural neurotransmitter, then it is said to be “positively linked” to that type of receptor. Conversely, if the undesirable mental or neurological condition is improved by the binding of the receptor to its natural neurotransmitter, then it is “negatively linked” to that type of receptor. For example, the undesirable mental or neurological condition of depression is negatively linked to serotonin post-synaptic receptors, because binding of these receptors to their natural neurotransmitter serotonin results in a decrease in the depression. The undesirable mental or neurological condition of depression is positively linked to kappa receptors, because binding of these receptors to their natural neurotransmitter dynorphin results in an increase in the depression.

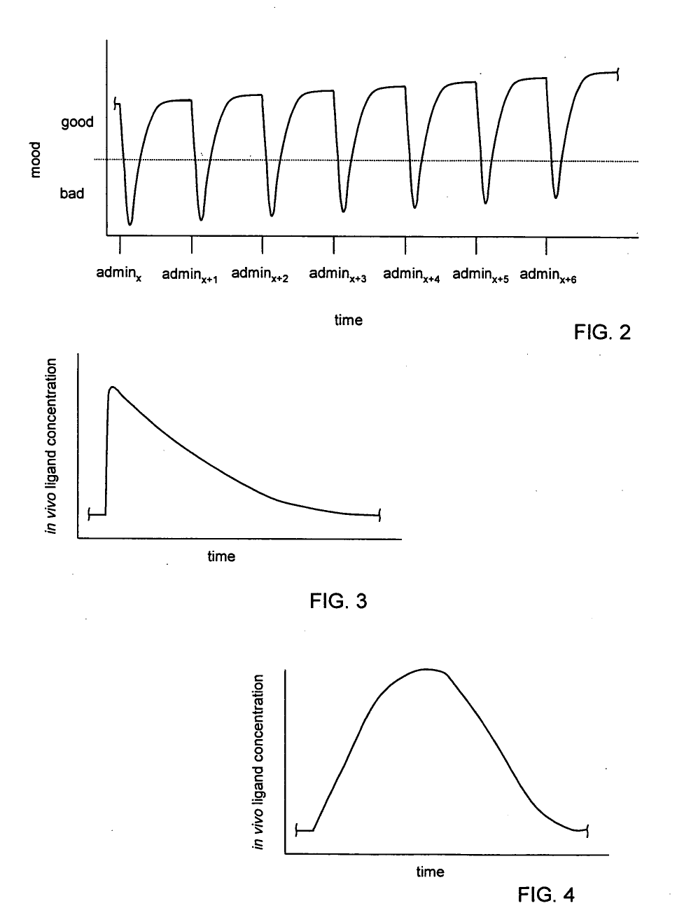

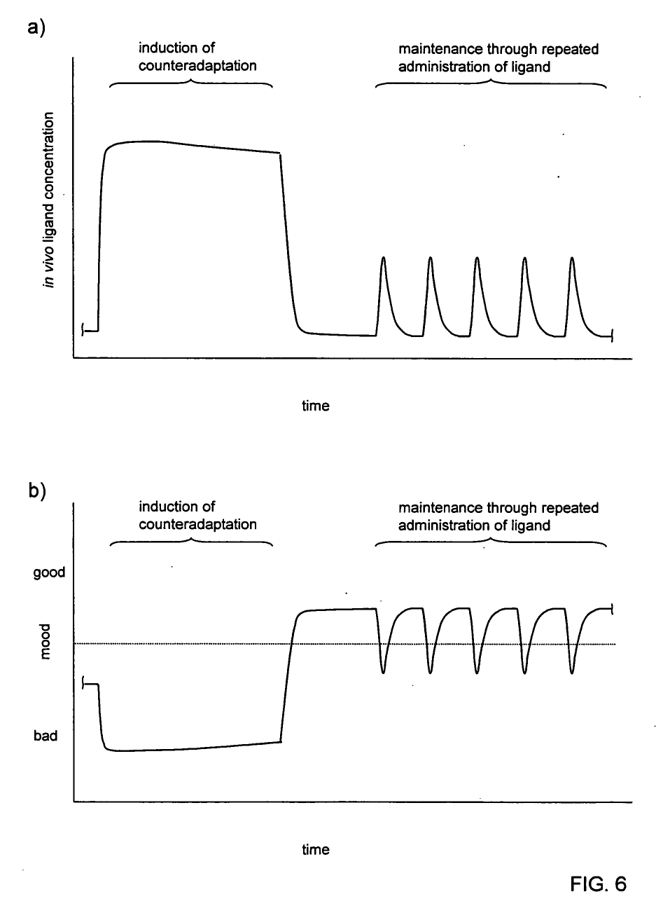

Instead of relying on the direct effect of ligand-receptor binding to regulate the neurotransmitter system, the methods of the present invention exploit the indirect counteradaptive effect to enhance or suppress neurotransmitter systems linked to undesirable mental or neurological conditions. The counteradaptation is the brain's natural response to the binding of the ligand. The initial effect of ligand binding may be a worsening of the undesirable mental or neurological condition. However, because the effects of the counteradaptation last long after the ligand is removed from the system, and can build up over repeated administration of the ligand, the counteradaptation causes an overall desirable regulation of the neurotransmitter system. The regulation of the neurotransmitter system can, in turn, provide a therapeutic benefit with respect to the undesirable mental or neurological condition. The regulation of the neurotransmitter system may be, for example, an increase in the counteradaptive response (as shown in FIG. 2, described below), or a maintenance of an already-induced counteradaptive response (as shown in FIG. 6, described below).

Counteradaptations are a manner by which the central nervous system maintains homeostasis. The counteradaptation is a result of the body's attempt to regulate the neurotransmitter system to its original steady-state level in order to prevent its over- or under-stimulation. Natural neurotransmitters bind with their receptors for only a short time, and are removed almost immediately from the synapse, and therefore do not cause a counteradaptive response. When a ligand interacts with a receptor for a longer period of time (e.g., because the ligand has a longer binding time or is continuously administered), however, cellular mechanisms gradually occur at the receptor/neurotransmitter level that act to counteract the direct effects of the ligand-receptor binding (i.e., the counteradaptation). The counteradaptation may be, for example, a change in the biosynthesis or release of a natural neurotransmitter that binds to the type of receptor, a change in the reuptake of a natural neurotransmitter that binds to the type of receptor, a change in the number of the type of receptors and/or binding sites on receptors of the type of receptor, a change in the sensitivity of receptors of the type of receptor to binding by the natural neurotransmitter and/or receptor agonists, or a combination thereof. Chronic use of a ligand thus induces (i.e., causes) a counteradaptation by stimulating processes that oppose the initial effects of the ligand, which over time results in a decrease in the effect of ligand-receptor binding.

When the ligand is a receptor agonist, the counteradaptation works to reduce the functioning of the neurotransmitter system (i.e., a “down-regulation”). The down-regulation may occur through, for example, a decrease in the biosynthesis or release of a natural neurotransmitter that binds to the type of receptor, an increase in the reuptake of a natural neurotransmitter that binds to the type of receptor, a decrease in the number of the type of receptors and/or binding sites on receptors of the type of receptor, a decrease in the sensitivity of receptors of the type of receptor to binding by the natural neurotransmitter and/or receptor agonists, or a combination thereof. Any of the above-recited counteradaptive responses will work to reduce the functioning of the neurotransmitter system, and can therefore provide a therapeutic benefit with respect to an undesirable mental or neurological condition that is positively linked to the neurotransmitter system.

Conversely, when the ligand is a receptor antagonist, the counteradaptation works to increase the functioning of the neurotransmitter system (i.e., an “up-regulation”). The up-regulation may occur through, for example, an increase in the biosynthesis or release of a natural neurotransmitter that binds to the type of receptor, a decrease in the reuptake of a natural neurotransmitter that binds to the type of receptor, an increase in the number of the type of receptors and/or binding sites on receptors of the type of receptor, an increase in the sensitivity of receptors of the type of receptor to binding by the natural neurotransmitter and/or receptor agonists, or a combination thereof. Any of the above-recited counteradaptive responses will work to increase the functioning of the neurotransmitter system, and therefore provide a therapeutic benefit with respect to an undesirable mental or neurological condition that is negatively linked to the neurotransmitter system.

Receptors in the brain are commonly regulated by a pre-synaptic negative inhibition control loop. Thus, for mood-elevating post-synaptic receptors (i.e., receptors negatively linked to an undesirable mental or neurological condition), it is desirable to use repeated agonist treatment at the associated inhibitory pre-synaptic receptors. Repeated agonist administration at a pre-synaptic inhibitory receptor results in a down-regulation of that receptor, lessening its inhibitory response and thereby increasing neural firing at the mood-elevating post-synaptic receptors and providing an elevation of mood.

An opposite strategy is desired for use with mood-depressing post-synaptic receptors (i.e., receptors positively linked to an undesirable mental or neurological condition). For such receptors, it is desirable to use repeated antagonist treatment at the associated inhibitory pre-synaptic receptors. Repeated antagonist administration at a pre-synaptic inhibitory receptor results in an up-regulation of that receptor, lessening its inhibitory response and thereby decreasing neural firing at the mood-depressing post-synaptic receptors and providing an elevation in mood.

The direct effect of the ligand binding during the first time period will often be an initial exacerbation of the undesirable mental or neurological condition. For example, when the administered ligand is an antagonist for a type of receptor linked negatively to the undesirable mental or neurological condition, the short-term effect of the binding is to block the receptors and prevent them from binding the natural neurotransmitter and firing. Similarly, when the administered ligand is an agonist for a type of receptor positively linked to the undesirable mental or neurological condition, the short term affect of the binding is to cause the receptors to fire. Both the firing of receptors positively linked to the undesirable mental or neurological condition and the prevention of the firing of receptors negatively linked to the undesirable mental or neurological condition can cause an initial worsening of symptoms. When the short-term effect of ligand-receptor binding wears off (e.g., due to the removal of ligand from the system), the counteradaptation remains to provide the desired regulation of the neurotransmitter system. Repeated administration can cause a gradually increasing regulation of neurotransmitter systems. In certain embodiments of the present invention described below, measures are taken to limit the effect on the patient of the direct effect of ligand-receptor binding.

FIG. 1 also includes in part (b) a graph of mood vs. time for administration of an appropriate ligand for a mood-associated receptor. As shown in the example of FIG. 1, the direct effect of ligand administration may be a worsening of mood during each first time period. This worsening of mood tapers off as the in vivo concentration of the ligand falls to its steady state level. After the ligand concentration returns to its low steady state level, the counteradaptation remains in place to provide an overall improvement in mood during a second time period associated with each administration and subsequent to the first time period. FIG. 2 is a graph of mood vs. time for several administrations of a ligand during a method according to the present invention. As evidenced in FIG. 2 by the ever-increasing mood (i.e., the graph generally slants up with time), the strength of the counteradaptation may build up with time, with each administration causing additional counteradaptive response. As such, an increasing therapeutic benefit may be realized with repeated intermittent administration of the ligand.

Each administration of the ligand has an administration half-life. As shown in the graph of part (a) of FIG. 1, the in vivo concentration of the ligand is at a relatively low baseline level at the beginning of the administration (e.g., the swallowing of a pill, the application of a transdermal patch, or the beginning of intravenous administration), then rises to some maximum level. After reaching a maximum, the in vivo concentration of the ligand will decrease back down to the baseline level (e.g., due to metabolism/excretion of the ligand), where it remains until the next administration. As shown in FIG. 1, the administration half-life is measured as the period of time between the beginning of the administration and the half-maximum point of the in vivo concentration as the concentration drops from its maximum level to the baseline level.

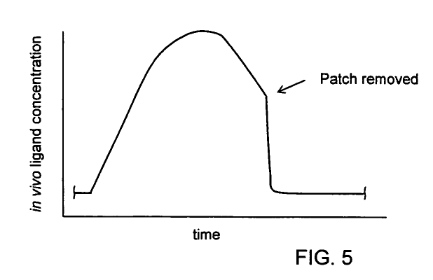

The administration half-life will be a function of the compound half-life (i.e., the half-life in vivo of the ligand compound itself) as well as of the route of administration. For example, FIG. 3 is a graph of in vivo concentration versus time for a single administration via injection of a ligand with a relatively long compound half-life. Because the injection gets the ligand into the bloodstream very quickly, the administration half-life approximates the compound half-life. In the example of FIG. 4, a ligand with a much shorter compound half life (e.g., a peptide) is administered using a time-release transdermal patch. Here, the concentration rises more slowly to a steady state maximum concentration, then falls off as the patch becomes depleted. Were the patch removed before depletion, the in vivo concentration would decrease rapidly down to the baseline level, as shown in FIG. 5. The administration half-life may be, for example, less than about a week, less than about three days, or less than about a day. More desirably, the administration half-life is less than about sixteen hours; less than about twelve hours, less than about eight hours; or less than about four hours. In certain embodiments of the invention, especially those using a ligand having a relatively long compound half-life, the administration half-life may be greater than about four hours; greater than about twelve hours; greater than about sixteen hours; or greater than about thirty hours.

The ligand has a compound half-life, defined as the in vivo half life of the ligand and its active metabolites (i.e., metabolites that are active at receptors of the type of receptor), divorced from any effects due to the route of administration. In certain embodiments of the present invention, it may be desirable to use a compound with a relatively short compound half-life. For example, in certain embodiments of the invention the compound half-life is less than about a week, less than about three days, or less than about a day. More desirably, the compound half-life is less than about sixteen hours; less than about twelve hours, less than about eight hours; or less than about four hours; or less than one hour. Some ligands, however, have relatively longer compound half-lives. For example, in certain embodiments of the invention, the compound half-life of the ligand is greater than about four hours; greater than about twelve hours; greater than about sixteen hours; or greater than about thirty hours.

The period between administrations is desirably selected so as to maximize the counteradaptive response to the ligand while maintaining an acceptably low and tolerable direct effect of ligand-receptor binding. For example, the administration of the ligand may be performed daily. In other embodiments of the invention, the period between administrations is two days or greater; three days or greater; five days or greater; one week or greater; two weeks or greater; or one month or greater. Similarly, the dose of the ligand at each administration is selected to be sufficient to trigger a counteradaptive response, but low enough that direct effects of ligand-receptor binding are low and tolerable to the patient.

When using a ligand having a compound half-life greater than about twelve hours, in order to increase the counteradaptation it may be desirable to repeatedly administer a second ligand for the type of receptor, with each administration of the second ligand having an administration half-life of less than about eight hours. In an example of a method according to the present invention, a ligand having a twenty-four hour compound half-life is administered every three days with a twenty four hour administration half-life, and a second ligand is administered daily with a six hour administration half-life. In such cases, when the ligand is a receptor agonist, the second ligand is desirably a receptor agonist; and when the ligand is a receptor antagonist, the second ligand is desirably a receptor antagonist.

The ratio of administration half-life to the period between administrations is desirably selected to maximize the counteradaptation while keeping any direct effects of ligand binding during the first time period at a low and tolerable level. According to one embodiment of the invention, the ratio of the administration half-life to the period between administrations is no greater than 1/2. Desirably, the ratio of the administration half-life to the period between administrations is no greater than 1/3. In certain embodiments of the invention, the ratio of the administration half-life to the period between administrations is no greater than 1/5; no greater than 1/8; or no greater than 1/12. It may be, however, desirable to administer the ligand relatively often, in order to maintain a desired level of counteradaptation. For example, in certain desirable embodiments of the invention the ratio of administration half-life to the period between administrations may be greater than 1/100; greater than 1/50; greater than 1/24; greater than 1/12; greater than 1/8; greater than 1/5; greater than 1/4; or greater than 1/3.

A substantial fraction of the receptors of the type of receptor are bound to the ligand during the first time period associated with each administration, so as to cause a counteradaptation to the ligand binding. For example, at least about 30%, at least about 50%, at least about 75%, or at least about 90% of the receptors of the type of receptor are bound by the ligand during each first time period.

Similarly, the first time period associated with each administration is desirably long enough to cause a substantial counteradaptation. For example, each first time period is desirably at least about five minutes in duration; at least about thirty minutes in duration; at least about an hour in duration; at least about two hours in duration; or at least about four hours in duration. In certain desirable embodiments of the invention, each first time period is about eight hours in duration. However, in cases where the direct effect of ligand binding is a noticeable worsening in the undesirable condition, it may be desirable to maintain the first time period no longer than necessary to get an acceptable level of counteradaptation. For example, in certain embodiments of the invention the first time period is desirably less than about twenty four hours in duration; less than about sixteen hours in duration; less than about twelve hours in duration; less than about eight hours in duration; or less than about six hours in duration.

In desired embodiments of the invention, a substantial fraction of the receptors remain unbound to the ligand during a second time period associated with each administration and subsequent to the first time period. A low level of ligand-receptor binding allows the patient to enjoy the effects (e.g., the therapeutic benefit) of the counteradaptation without interference from any ill effects of direct ligand binding. For example, desirably no more than about 50%, no more than about 25%, no more than about 10% of the receptors are bound to the ligand during each second time period.

The second time period associated with each administration is the time during which a substantial fraction of the receptors of the type of receptor are unbound to the ligand. During each second time period, the patient may enjoy any therapeutic benefit of the counteradaptation, as no direct ligand-receptor binding effects would remain. As such, each second time period is desirably as long as possible. For example, each second time period is desirably at least about two hours in duration; at least about ten hours in duration; or at least about fifteen hours in duration. However, it may be desirable to keep each second time period relatively short, in order to decrease the period between administrations thereby increase the counteradaptation. For example, in certain embodiments of the invention each second time period is desirably no more than about twenty hours in duration; no more than about thirty hours in duration; or no more than about fifty hours in duration.

In order to build up a counteradaptation over time and to minimize any initial exacerbation of the undesirable mental or neurological condition, it may be desirable to begin the treatment with a relatively low dose of ligand at each administration, and increase the dosage over time. Increasing dosages can also be used to account for any tolerance the patient builds up to the ligand. For the sake of convenience, it may be desirable to increase the dosage intermittently over time (i.e., increase the dosage with a period longer than the period between administrations). For example, in certain embodiments of the invention the dosage is increased with a period between increases of no less than a week; no less than two weeks; no less than three weeks; no less than a month; no less than two months; no less than three months; no less than six months, or no less than one year. At each increase in dosage, the dose is desirably increased by at least about 5%; at least about 10%; at least about 25%; at least about 50%; or at least about 100% of the initial dose. It may, however, be desirable to maintain the maximum dosage within certain limits. For example, in certain embodiments of the invention the maximum dosage may be within three hundred times the initial dosage, within one hundred times the initial dosage, within fifty times the initial dosage, or within twenty times the initial dosage.

In one example of a dosing schedule, low doses of a ligand are given for one, two, or three weeks. These initial doses are high enough to induce a counteradaptive response, but low enough to cause only minimal direct effects due to ligand-receptor binding. The dose is then increased. The increase may be as small as 10%; for more rapid induction of a counteradaptive response, however, it is desirable to at least double the initial dose. After four to six weeks the dosage is again increased. This pattern is followed every one, two, four or six months. The endpoint for the maximum dosage will depend on individual tolerance to the ligand and the development of side effects and direct effects from the larger doses.

To reduce the impact of any direct effects of ligand-receptor binding, it may be desirable to time the administration of the ligand so that the first time period occurs during a time when adverse effects on the patient will be minimized. The patient will not notice a decrease in mood if she is asleep. For example, it may be desirable to time the administration of the ligand so that a substantial fraction of the first time period occurs while the patient is asleep, so that any direct effects of ligand-receptor binding are not noticed. For example, at least 40%; at least 60%; or at least 85% of the first time period desirably occurs while the patient is asleep. In order to achieve such timing, it may be desirable to perform a substantial fraction of the administrations of the ligand within the hour before the patient goes to bed. For example, desirably at least 50%; at least 75%; at least 90%; or at least 95% of the administrations of the ligand are performed within the hour before the patient goes to bed.

There is no contraindication for daytime administration, however, and in other embodiments of the invention, each administration of the ligand is performed more than one hour before the patient goes to bed. In one example of a method according to the present invention, a patient who has been administered a ligand daily for two or three months and has developed a counteradaptation and some associated improvement in mood. If there were a particular time of day the patient wanted to enhance daytime mood, the time of ligand administration could be moved so that the desired time would fall within the second time period associated with that administration. If the patient wanted an elevated mood at 6 p.m., he could administer an appropriate ligand (e.g., naloxone, a mu and/or delta opiate receptor antagonist with a compound half-life of one hour) at 2 p.m. The direct effect of naloxone-receptor binding (a bad mood) would last only a couple of hours, leaving only the good mood caused by the counteradaptation by 6 p.m.

The administration of the ligand is desirably repeated enough to build up a suitably large counteradaptive effect. As such, in the methods of the present invention, the administration is desirably performed at least five times, at least ten times, at least twenty-five times, or at least fifty times.

Each administration of the ligand may be performed orally, transdermally, through inhalation, subcutaneously, intravenously, intramuscularly, intraspinally, intrathecally, transmucosally, or using an osmotic pump, a microcapsule, an implant or a suspension. The skilled artisan will select the route of administration based upon the identity of the ligand, its compound half-life, the desired dose and the desired administration half-life.

It may be desirable to administer the ligand using both a rapidly absorbed loading dose (in order to get a fast ligand-receptor binding), and a gradually absorbed dose (in order to maintain a desired level of ligand-receptor binding over the desired length of the first time period). A rectal suppository having a rapidly-absorbing outer covering and a more slowly absorbing center could be used for such an administration. Alternatively, the loading dose could be given sublingually, and the gradually absorbed dose could be given transdermally via patch.

A carrier in the blood may be used to increase the administration half-life of the ligand once it is in circulation. For example, U.S. Pat. Nos. 6,610,825 and 6,602,981, each of which is incorporated herein by reference in its entirety, describe a method by which ligands are bound to blood cells or proteins in order to extend their administration half-life. Adessi et al (Curr Med Chem, 9(9); May, 2002; 963-978) describe a method by which to stabilize peptide ligands.

The undesirable mental or neurological condition may be any condition linked to the neurotransmitter system. Examples of such conditions include chronic pain, mood disorders, eating disorders, anxiety disorders, motivational and performance problems, inflammatory conditions, nausea, emesis, urinary incontinence, skin rashes, erythema, and eruptions. More examples of undesirable mental or neurological conditions are described below.

It may also be desirable to administer an anxiolytic agent in combination with the ligand, so as to reduce any direct effects of ligand-receptor binding. The anxiolytic agent may especially help mitigate the effects of ligand-receptor binding on the patient's sleep. The anxiolytic agent may, for example, affect a GABA pathway. The anxiolytic agent may be, for example, a benzodiazepine such as diazepam, lorazepam, alprazolam, temazepam, flurazepam, and chlodiazepoxide. Similarly, it may be desirable to administer a hypnotic agent or a selective serotonin reuptake inhibitor in combination with the ligand, so as to reduce any direct effects of ligand-receptor binding. Each of these agents may be administered at the same time as the ligand, or at a different time. It may also be desirable to add tryptophan to the patient's diet, as described in U.S. Pat. Nos. 4,377,595 and 5,958,429, each of which is incorporated herein by reference in its entirety.

It may be desirable to administer conventional pharmaceutical agents in combination (e.g., simultaneously or sequentially) with the ligand. Administration of such an agent is especially desirable when it is an agonist for a type of receptor that has been increased in number and/or sensitivity through a counteradaptation, or is an antagonist for a type of receptor that has been decreased in number and/or sensitivity through a counteradaptation. Examples of conventional pharmaceutical agents that may administered in combination with the ligand include TCAs, MAOIs, SSRIs, NRIs, SNRIs, CRF modulating agents, serotonin pre-synaptic autoreceptor antagonists, 5HT₁ agonist, dynorphin antagonists, GABA-A modulating agents, serotonin 5H_{2C} and/or 5H_{2B} modulating agents, beta-3 adrenoceptor agonists, NMDA antagonists, V1B antagonists, GPCR modulating agents, or substance P antagonists. Desirably, the additional pharmaceutical agent has a relatively short administration half-life, so that it can be administered during the second time period, with its effect substantially absent by the next administration of the ligand. Such an administration regimen maintains a high level of counteradaptation, while maximizing the effect of the pharmaceutical agent during the second time period.

It may also be desirable to take advantage of direct binding of the receptors to provide a desired clinical effect. For example, when the ligand is a receptor agonist, it may be desirable to administer an antagonist for the type of receptor during one or more of the second time periods associated with each administration and subsequent to the first time period. However, the antagonist for the type of receptor is desirably not administered during the first time period associated with each administration. Similarly, when the ligand is a receptor antagonist, it may be desirable to administer an agonist for the type of receptor during one or more of the second time periods associated with each administration and subsequent to the first time period. However, the agonist for the type of receptor is desirably not administered during the first time period associated with each administration. Preferably the antagonist has an in vivo half life of less than 12 hours, less than 8 hours, or less than 6 hours, such that it would not interfere with the subsequent administration of the agonist.

Another embodiment of the present invention is illustrated by the graphs of in vivo ligand concentration (part a) and mood vs. time (part b) of FIG. 6. In this method, a counteradaptation is first induced by giving the patient one or more doses of a ligand for the type of receptor. As shown in FIG. 6, this could be through repeated or continuous administration of high doses of the ligand. Relatively high, long-term doses of the ligand will induce a strong counteradaptive effect, but may cause the patient to suffer marked direct effects from ligand-receptor binding, as shown in the graph of mood vs. time of FIG. 6. In such cases, it may be desirable to keep the patient hospitalized during the initial induction of the counteradaptive response. After the counteradaptive response is induced, it is maintained repeatedly administering the ligand to the patient with a ratio of administration half-life to period between administrations no greater than 1/2. The repeated administration may be performed substantially as described above.

Through regulation of the function of neurotransmitter systems, the methods of the present invention may be used to improve undesirable mental and neurological conditions, even if they are not able to cure them. The methods of the present invention may make undesirable mental and neurological conditions more amenable to conventional therapies. For example, even if clinical depression is not cured, the improved mood caused by the use of the methods of the present invention may help improve the depression. As described above, the use of conventional antidepressants may also be made more efficacious. In another example, even if cancer is not cured, the regulation of the neurotransmitter acts to suppress tumor growth and/or metastasis, and may make conventional cancer therapies and/or the immune system better able to eliminate the cancerous growth. The therapeutic benefits caused by the regulation of the neurotransmitter may be, for example, a decrease in the severity of the symptoms associated with the mental and neurological condition; an eradication of the symptoms associated with the mental and neurological condition; or an increase in a mood that masks the symptoms associated with the mental and neurological condition.

The methods according to the present invention may be used therapeutically to address an undesirable mental or neurological condition in a patient. For example, the methods of the present invention may be used to treat a pre-existing undesirable mental or neurological condition in a patient. The methods may also be used to reduce any future undesirable mental or neurological condition that is anticipated to occur, for example, due to future physical exertion, physical trauma, mental trauma, or medical procedure.

### The Substance P System

According to one embodiment of the invention, the neurotransmitter system is the Substance P (“SP”) system which includes as neurotransmitters the neurokinins Substance P, NKA and NKB. SP is a polypeptide and is known to act as a neurotransmitter and mediator for pain sensations. It is a member of the tachykinin family, which is a set of polypeptides having a similar C-terminal and a varying N-terminals with varying SP-like activity. The SP receptors include NK-1, NK-2 and NK-3 receptors. SP preferentially binds to NK-1 receptors, NKA preferentially binds to NK-2 receptors, and NKB preferentially binds to NK-3 receptors.

SP and its receptors are found primarily in the brain and spinal cord tissue. In the spinal cord, SP receptors are found in an area called the dorsal horn, which is a primary site for pain signals to be transmitted to the brain. In the brain, SP and its receptors are found in large concentrations in the hypothalamus and the amygdala, areas associated with affective behavior, anxiety and response to stress, and pain. In addition, SP is also implicated in nausea and emesis, defensive behavior, cardiovascular tone, salivary secretion, inflammation, smooth muscle contraction and vasodilation, as well as in numerous mental conditions such as schizophrenia, manic depressive psychosis, sexual dysfunction, drug addiction, cognitive disorders, locomotive disorders, and depression.

When the neurotransmitter system is the SP system, the type of receptor is SP receptors, which are positively linked to undesirable mental and neurological conditions, and the ligand is an SP receptor agonist. The counteradaptation causes a down-regulation of the SP system, and may be at least one of a decrease in the biosynthesis or release of SP, NKA and/or NKB at the receptor terminals or by the pituitary gland; a decrease in the number of the receptors and/or binding sites on the receptors; or a decrease in the sensitivity of the receptors to binding by SP receptor agonists and/or SP, NKA and/or NKB.

The SP receptor agonist may be, for example, peptide-based. In certain embodiments of the invention, the SP receptor agonist is an analogue of SP, NKA, and/or NKB, or a pharmaceutically acceptable salt or derivative thereof. For example, the SP receptor agonist may be Substance P; Substance P, free acid; Biotin-Substance P; [Cys^{3,6}, Tyr⁸, Pro⁹]-Substance P; (Disulfide bridge: 3-6), [Cys^{3,6}, Tyr⁸, Pro¹⁰]-Substance P; (Disulfide bridge: 3-6), [4-Chloro-Phe^{7,8}]-Substance P; [4-Benzoyl-Phe⁸]-Substance P; [Succinyl-Asp⁶, N-Me-Phe⁸]-Substance P (6-11)(Senktide); [Tyr⁸]-Substance P; [Tyr⁹]-Substance P; Shark Substance P Peptide; GR73632 [D-Ala-[L-Pro⁹,Me-Leu⁸]substance P(7-11)]; [Sar⁹,Met(O₂)¹¹]SP; GR 73,632 [delta-Aminovaleryl [Pro9, N-Me-Leu10]-substance P(7-11)], [Glu(OBzl)11]substance P and hemokinin 1 (HK-1) (a substance P homolog); or a pharmaceutically acceptable salt or carrier thereof.

In other embodiments of the invention, the SP receptor agonist may be an NKA or NKB analogue having a C-terminal heptapetpide similar to NKA(4-10) or NKB(4-10), or a pharmaceutically acceptable salt or carrier thereof. For example, the SP receptor agonist may be [Gln⁴]-NKA, [Gln⁴]-NKA(4-10), [Phe⁷]-NKA, [Phe⁷]-NKA(4-10), [Ile⁷]-NKA, [Ile7]-NKA(4-10), [Lys⁵,MeLeu⁹,Nle¹⁰]-NKA(4-10), [Nle¹⁰]-NKA(4-10), β-Ala⁸]-NKA(4-10), [Ala⁵]-NKA(4-10), *[Gln⁴]-NKB, [Gln⁴]-NKB(4-10), [Phe⁷]-NKB, [Phe⁷]-NKB(4-10), [Ile⁷]-NKB, [Ile7]-NKB(4-10), [Lys⁵,MeLeu⁹,Nle¹⁰]-NKB(4-10), [Nle¹⁰]-NKB(4-10), β-Ala⁸]-NKB(4-10), [Ala⁵]-NKB(4-10), or a pharmaceutically acceptable salt or carrier thereof. Similarly, the SP receptor agonist may be [Arg]-NKB, an NKA or NKB analogue having Val⁷ replaced with MePhe, or a pharmaceutically accepted salt or carrier thereof.

Other SP receptor agonists that may be used in the present invention are SR 48968, an NK2 receptor antagonist ((S)-N-methyl-N [4-(4-acetylamino-4-[phenyl piperidino)-2-(3,4-dichlorophenyl)-butyl]benzamide]) as well as those described in U.S. Pat. Nos. 4,839,465; 4,472,305; 5,137,873; 4,638,046; 4,680,283; 5,166,136; 5,410,019; and 6,642,233, each of which is incorporated herein by reference in its entirety.

The initial dosage (i.e., the dosage at the first administration) of the SP receptor agonist is desirably high enough to induce a counteradaptive effect, but not so high as to cause intolerable direct effects from ligand-receptor binding. For example, the initial dosage of the SP receptor agonist may be between about 0.5 pmol/kg/min and about 20 pmol/kg/min for continuous dosing during the first time period. In certain desirable embodiments of the invention, the initial dosage of the SP receptor agonist is between 3 pmol/kg/min and 10 pmol/kg/min for continuous dosing during the first time period.

The present invention is not limited to the use of peptide-based SP receptor agonists. Other SP receptor agonists, including substantially or wholly non-peptidic SP receptor agonists (e.g., those described in Chorev et al., Biopolymers, May 1991; 31(6):725-33), which is hereby incorporated herein by reference in its entirety) may be used in the methods of the present invention.

The SP receptor agonist may be administered using any appropriate route. Transmucosal administration is an especially desirable method for administering SP receptor agonists. For example, the administration may be sublingual or via rectal suppository. It may be desirable to administer the SP receptor agonist using both a rapidly absorbed loading dose (in order to get a fast binding of the SP receptors), and a gradually absorbed dose (in order to maintain a desired level of agonist-receptor binding over the desired length of the first time period). A rectal suppository having a rapidly-absorbing outer covering and a more slowly absorbing center could be used for such an administration. Alternatively, the loading dose could be given sublingually, and the gradually absorbed dose could be given transdermally via patch. Other routes include intraspinal or intrathecal administration for pain.

Desirably, an SP receptor antagonist is not administered during the first time period associated with each administration. In certain embodiments of the invention, however, an SP receptor antagonist is administered during one or more of the second time periods. Non-limiting examples of SP receptor antagonists along with suggested dosages are as follows: SR 48968 ((S)-N-methyl-N[4-(4-acetylamino-4-[phenyl piperidino)-2-(3,4-dichlorophenyl)-butyl]benzamide]); Osanetant and compounds described in U.S. Pat. Nos. 5,972,938; 6,576,638; 6,596,692; 6,509,014; 6,642,240; 6,841,551; 6,177,450; 6,518,295; U.S. Pat. No. 6,369,074; AND U.S. Pat. No. 6,586,432; AND WO 95/16679; 95/18124; 95/23798.

Other SP(NK₁) receptor antagonists include: L-760735 ([1-(5-{[(2R,3S)-2-({(1R)-1-[3,5-bis(trifluoromethyl)phenyl]ethyl}oxy)-3-(4-phenyl)morpholin-4-yl]methyl}-2H-1,2,3-triazol-4-yl)-N,N-dimethylmethanamine]) (See Boyce, S, et al. Neuropharmacology. 2001 July; 41(1):130-7); CP-96,345 [(2S,3 S)-cis-2-(diphenylmethyl)-N-[(2-methoxy-phenyl)-methyl]-1-azabicyclo[2.2.2]-octan-3-amine] (See Snider, et al, Science, 1991 Jan. 25; 251(4992):435-7); SSR240600 ([(R)-2-(1-{2-[4-{2-[3,5-bis(trifluoromethyl)phenyl]acetyl}-2-(3,4-dichlorophenyl)-2-morpholinyl]ethyl}-4-piperidinyl)-2-methylpropanamide] (See Steinberg, R. et al., Steinberg, R, et al, J Pharm Exper Ther, 303(3), 1180-1188, December 2002, “SSR240600 [(R)-2-(1-{2-[4-{2-[3,5-Bis(trifluoromethyl)phenyl]acetyl}-2-(3,4-dichlorophenyl)-2-morpholinyl]ethyl}-4-piperidinyl)-2-methylpropanamide], a Centrally Active Nonpeptide Antagonist of the Tachykinin Neurokinin 1 Receptor: II. Neurochemical and Behavioral Characterization”); NKP608 [quinoline-4-carboxylic acid [trans-(2R,4S)-1-(3,5-bis-trifluoromethyl-benzoyl)-2-(4-chloro-benzyl)-piperidin-4-yl]-amide)] (see Spooren W P, et al., Eur J. Pharmacol. 2002 Jan. 25;435(2-3):161-70 and File, S E, Psychopharmacology (Berl). 2000 September; 152(1):105-9, entitled “NKP608, an NK1 receptor antagonist, has an anxiolytic action in the social interaction test in rats.”); L-AT (N-acetyl-L-tryptophan 3,5-bis benzyl ester) (See Crissman, A, et al., Vol. 302, Issue 2, 606-611, August 2002, entitled “Effects of Antidepressants in Rats Trained to Discriminate Centrally Administered Isoproterenol”); MK-869 [Aprepitant] (See Varty, G B, et al., Neuropsychopharmacology (2002) 27 371-379, “The Gerbil Elevated Plus-maze II: Anxiolytic-like Effects of Selective Neurokinin NK1 Receptor Antagonists”); L-742,694 [2(S)-((3,5-bis(Trifluoromethyl)benzyl)-oxy)-3(S)phenyl-4-((3-oxo-1,2,4-triazol-5-yl)methyl)morpholine] (See Varty, et al.); L-733060 [(2S,3S)-3-([3,5-bis(trifluoromethyl)phenyl]methoxy)-2-phenylpiperidine] (See Varty, et al.); CP-99,994 [(+)-(2S,3 S)-3-(2-methoxybenzylamino)-2-phenylpiperidine] (See McLean, et al, J Pharm Exp Ther, Volume 267, Issue 1, pp. 472-479 and Varty, et al.); CP-122,721 [(+)-(2S,3S)-3-(2-methoxy-5-trifluoromethoxybenzyl)amino-2-phenylpiperidine] (See McLean, et al., J Pharm ExpTher, Volume 277, Issue 2, pp. 900-908 and Varty, et al); CP-96,345 [(2S,3 S)-cis-2-(diphenylmethyl)-N-((2-methoxyphenyl)-methyl)-1-azabicyclo(2.2.2.)-octan-3-amine] (see Bang, et al., J Pharmacol Exp Ther. 2003 April; 305(1):31-9); GSK 597599 [Vestipitant]; GSK 679769 (See Hunter et al. U.S. Patent Publication no. U.S. Pat. No. 2,005,0186245); GSK 823296 (See Hunter et al. U.S. Patent Publication no. 20050186245); Saredutant (See Van Schoor, et al., Eur Respir J 1998; 12: 17-23; Talnetant; Osanetant (see Kamali, F, Curr Opin Investig Drugs. 2001 July; 2(7):950-6); SR-489686 (benzamide, N-[4-[4-(acetylamino)-4-phenyl-1-piperidinyl]-2-(3,4-dichloro-phenyl)butyl]-N-methyl-(S)-); SB-223412 (See Hunter et al. U.S. Patent Publication no. 20050186245); SB-235375 (4-quinolinecarboxamide-, 3-hydroxy-2-phenyl-N-[(1S)-1-phenylpropyl]-), UK-226471 (See Hunter et al. U.S. Patent Publication no. 20050186245).

Suitable but non-limiting initial dosages for SP receptor antagonists include about 12 mg/kg/hour/administration for 8 hours of L-760735 (via iv); about 30 ug/kg/hour/administration for 8 hours of CP-96,345 (via iv); between about 0.1 to 10 mg/kg/administration of SSR240600 (via ip or po); between about 0.01 to 0.1 mg/kg/administration of NKP608 (via po); between about 1 to 10 mg/kg/administration of L-AT; between about 0.01 to 3 mg/kg/administration of MK-869; between about 1 to 30 mg/kg of L-742,694; between about 1 to 10 mg/kg/administration of L-733,060; between about 3 to 30 mg/kg/administration of CP-99,994 or CP-122,721; and about 100 mg/administration of Saredutant (via po).

The SP neurotransmitter system is positively linked to a wide variety of undesirable mental and neurological conditions. Examples of such conditions include chronic pain, mood disorders, eating disorders, anxiety disorders, motivational problems, substance abuse disorders, inflammatory conditions, nausea or emesis (e.g., arising from chemotherapy), urinary incontinence, skin rashes, erythema, eruptions, fibromyalgia, chronic fatigue syndrome, chronic back pain, chronic headaches, chronic cancer pain, shingles, reflex sympathetic dystrophy, neuropathy, inflammatory pain, pain that is anticipated to occur in the future (e.g., from a medical procedure or physical exertion), major depressive disorders, post-traumatic depression, temporary depressed mood, manic-depressive disorder, dysthymic disorder, generalized mood disorder, anhedonia, non-organic sexual dysfunction, overeating, obesity, anorexia, bulimia, generalized anxiety state, panic disorders, phobias, obsessive-compulsive disorder, attention deficit hyperactivity disorder, Tourette's Syndrome, hysteria sleep disorders, breathing-related sleep disorders, a lack of motivation due to learning or memory problems, abuse of substances such as narcotics, alcohol, nicotine, stimulants, anxiolytics, CNS depressants, hallucinogens and marijuana, asthma, arthritis, rhinitis, conjunctivitis, inflammatory bowel disease, inflammation of the skin or mucosa, acute pancreatitis. The down-regulation of the SP system desirably causes a therapeutic benefit with respect to the undesirable mental or neurological condition.

Virtually all types of pain, with the exception of acute sharp pain, are associated with the SP system. SP is not involved with the initial pain that is caused by a stabbing wound. The pain that lingers afterwards, however, is due to the SP pathway. In a similar manner the pain that lingers for a period of time after a surgical procedure is mediated by the SP pathway.

Mood is mediated through the SP system. Increased levels of SP are found in clinically depressed patients. Substance abusers have elevated levels of SP and, for those times when they are not on the abused substance, generally have a depressed and/or dysphoric mood. Clinical depression and substance abuse are thus both associated with an up regulation of the SP system. The pleasurable experiences of morphine are absent in mice that lack the SP receptor. Such mice do not become addicted to morphine (Murtra, et al., Nature 405, 180-183, May 11, 2000). Because opiates alone cannot induce euphoria, the Murtra study suggests that the SP system is the final pathway by which opiate euphoria is mediated. The fact that SP antagonists can acutely improve mood is consistent with this finding. Anxiety, response to stress, sexual dysfunction and eating disorders are largely related to mood, and are therefore also affected by the SP system.

The SP system has also been implicated in asthma (Kudlacz E. M., “Combined tachykinin receptor antagonists for the treatment of respiratory diseases”, Expert Opinion on Investigational Drugs, Vol. 7, No. 7, July 1998, pp. 1055-1062) nausea/emesis, cancerous tumor growth and metastasis (Palma, C, et al., Br. J. Cancer, 1999 January; Vol. 79(2): 236-43 and Friess, et al., Lab. Invest. 2003 May; Vol. 83(5):731-42), and urinary incontinence (Andersson K E, Experimental Physiology, Vol. 84(1), 195-213).

Methods of the present invention using SP receptor agonists as ligands may be used to address undesirable mental or neurological conditions in patients. For example the methods of the present embodiment of the invention may be used to address any of the above-listed conditions. The methods according to the present embodiment of the invention may also be used as an adjunct treatment for cancer (e.g., to decrease tumor growth and metastasis).

The methods of the present invention could also be used with an SP agonist in chronic recurring pain situations such as migraine headaches. Similarly, because the SP system is up-regulated in chronic pain syndromes, they may also be treated using the methods of the present invention with an SP agonist. Such chronic pain syndromes include pain due to nerve injury, neuropathies, chronic low back pain, reflex sympathetic dystrophy, cancer pain, shingles and arthritis.

The methods of the present invention can be used with SP agonists in the prophylaxis of pain prior to an event that is associated with pain. The methods of the present invention may be used in order to decrease post-operative pain and also to increase post-operative response to narcotic pain medications, which results in a lower dose of narcotics to obtain an analgesic effect. Similarly, an SP agonist could be used in the methods of the present invention prior to such pain-inducing competitive events such as football, hockey, and boxing. An SP agonist could be used prior to any competitive event, such as long distance running in order to reduce pain perceptions that are inevitable with such muscle and leg overuse activities. A reduced pain response ultimately allows the athlete to push him/her self to a greater extent, resulting in an improved performance.

The methods of the present invention may also be used with SP agonists in order to address anxiety, stress response, sexual dysfunction and eating disorders may be improved with the SP agonist CAT protocol. These conditions are largely related to mood, thus an improvement in conditions such as these are indirectly related to overall mood as opposed to a direct effect.

The methods of the present invention may also be used with SP agonists in order to address any or all addictive disorders. For example, the methods of the present invention can be used to address the abuse of substances such as narcotics, alcohol, nicotine/cigarettes, stimulants, anxiolytics, CNS depressants, hallucinogens and marijuana. Furthermore, gambling and electronic gaming addictions follow the same brain abnormalities as do substance abuse problems, and can also be addressed using the methods of the present invention.

The methods of the present invention may also be used with SP agonists in order to address asthma by decreasing the severity of asthma attacks. An inhalational route of administration may be used in order to concentrate the counteradaptive effect in the lungs where it is most needed. The methods of the present invention may also be used with SP agonists in order to decrease the inflammatory response in any one of a number of inflammatory conditions such as arthritis, rhinitis, conjunctivitis, inflammatory bowel disease, inflammation of the skin and mucosa and acute pancreatitis. The methods of the present invention may also be used with SP agonists in order to address nausea/emesis, especially that associated with chemotherapy for cancer, and urinary incontinence.

### The Endogenous Endorphin System

According to another embodiment of the invention, the neurotransmitter system is the endogenous endorphin system, which includes as neurotransmitters the endorphins that bind preferentially to mu and/or delta opiate receptors. Endorphins are endogenous opiate-like compounds that act through their effects on the binding of opiate receptors. Mu and delta opiate receptors act in unison, and are stimulated by opiate and opiate-like compounds. Mu receptors primarily modulate pain, but also modulate mood. Delta receptors have the opposite focus, primarily modulating mood, but also modulating pain.

When the neurotransmitter is the endogenous endorphin system, the type of receptor is mu and/or delta opiate receptors, which are negatively linked to undesirable mental and neurological conditions. Mu opiate receptors are associated primarily with lower levels of pain when stimulated, while delta opiate receptors are associated primarily with euphoria when stimulated. The ligand is a mu and/or delta opiate receptor antagonist, and the counteradaptation causes an up-regulation of the endogenous endorphin system. The counteradaptation may be, for example, an increase in the biosynthesis or release of endorphins at receptor terminals and/or by the pituitary gland; an increase in the number of the receptors and/or endorphin binding sites on the receptors; an increase in the sensitivity of the receptors to binding by mu and/or delta receptor agonists and/or endorphins; or a combination thereof.

The method according to the present embodiment of the invention may be practiced using a specific mu receptor antagonist or a specific delta receptor antagonist. For example, the method may be practiced using a specific mu receptor antagonist such as clocinnamox mesylate, CTAP, CTOP, etonitazenyl isothiocyanate, β-funaltrexamine hydrochloride, naloxonazine dihydrochloride, Cyprodime, and pharmaceutically acceptable salts, analogues, and derivatives thereof. The method may also be practiced using specific delta receptor antagonists such as naltrindole, N-benzylnaltrindole HCl, BNTX maleate, BNTX, ICI-154,129, ICI-174,864 (N,N-diallyl-Tyr-Aib-Aib-Phe-Leu-OH, where Aib is alpha-amino-isobutyric acid), naltriben mesylate, SDM25N HCl, 7-benzylidenenaltrexone, and pharmaceutically acceptable salts, analogues, and derivatives thereof. The skilled artisan may also employ non-specific mu and/or opiate antagonists, such as naloxone and naltrexone, in the methods according to the present embodiment of the invention. Non-limiting representative examples of non-specific opiate antagonists include Nalorphine, nalbuphine, levallorphin, cyclazocine, diprenorphine

Other mu and/or delta opiate receptor antagonists useable in the methods of the present invention include those described in U.S. Pat. Nos. 5,922,887; 4,518,711; 5,332,818; 6,790,854; 6,770,654; 6,696,457; 6,552,036; 6,514,975; 6,436,959; 6,306,876; 6,271,239; 6,262,104; 5,552,404; 5,574,159; 5,658,908; 5,681,830; 5,464,841; 5,631,263; 5,602.099; 5,411,965; 5,352,680; 5,332,818; 4,910,152; 4,816,586; 4,518,711; 5,872,097; 5,821,219; 5,326,751; 4,421,744; 4,464,358; 4,474,767; 4,476,117; 4,468,383; 6,825,205; 6,455,536; 6,740,659; 6,713,488; 6,838,580; 6,337,319; 5,965,701; 6,303,578; and 4,684,620, each of which is incorporated herein by reference in its entirety.

In certain desirable embodiments of the invention, the mu and/or delta opiate receptor antagonist is naloxone, naltrexone, nalmefene, or nalbuphine, or a pharmaceutically acceptable salt or derivative thereof. Naltrexone is a desirable mu and/or delta receptor antagonist, but may not be usable in all situations due to its long compound half-life (48-72 hours); while naltrexone itself has a half-life of 9-10 hours, its active metabolites (e.g. 6-beta-naltrexol and 2-hydroxy-3-methoxynaltrexol) have much longer half-lives. Naloxone is an especially desirable mu and/or delta receptor antagonist for use in the present embodiment of the invention. Naloxone has a compound half-life of about an hour, but cannot be given orally. Naloxone can be given intravenously or through a transdermal patch, desirably using a time-release formulation. Suitable transdermal patches are described in U.S. Pat. No. 4,573,995, which is hereby incorporated herein by reference in its entirety.

The initial dosage of the mu/and or delta opiate receptor is desirably high enough to induce a counteradaptive effect, but not so high as to cause the patient intolerable direct effects. For example, the initial dosage of the mu and/or delta opiate receptor antagonist may be equivalent to between about 2 mg/administration and about 200 mg/administration of naloxone. In certain desirable embodiments of the invention, the initial dosage of the mu and/or delta opiate receptor antagonist is equivalent to between about 10 mg/administration and about 100 mg/administration of naloxone.

When using naloxone as the mu and/or delta opiate receptor antagonist, the initial dosage may be between 5 and 500 mg/administration. Desirably, the initial dosage is between 10 and 50 mg/administration. In certain embodiments of the invention, each dosage of naloxone is greater than 10 mg/administration; greater than 10.5 mg/administration; greater than 11 mg/administration; or greater than 15 mg/administration. Desirably, the initial dose of naloxone is at least about 30 mg/administration (over 8 hour period), as this amount results in a complete blockade of opiate receptors. Desirably, the maximum dosage of naloxone is no greater than 3000 mg/administration.

In one example of a daily dosing regimen for naloxone, the initial dosage of naloxone is 30 mg/administration over an 8 hour period. After two weeks, the dosage is doubled. After another two weeks, the dosage is increased to 120-160 mg/administration. After another month, the dosage is increased to 300 mg/administration, then to 500-600 mg/administration after another two months. After another two months, the dosage is increased to 1000 mg/administration, then to 1500-2000 mg/administration after another two months. Alternatively, a much larger initial dose (e.g., 100-500 mg/administration) could be used in order to build up a counteradaptation more quickly. A low dose of naltrexone (e.g., 10-25 mg/administration) could be used along with the naloxone to realize an additional counteradaptive effect.

In one example of a dosing regimen for naltrexone, an initial dosage of 10-25 mg naltrexone is given daily. Alternatively, larger doses (e.g., 25-200 mg/administration) are given once, twice, or thrice weekly. With larger doses of naltrexone, the first time period will be relatively long, and may occasionally overlap with the waking hours of the patient.

The mu and/or delta opiate receptor antagonist may be administered orally, transdermally, intraspinally, intrathecally, via inhalation, subcutaneously, intravenously, intramuscularly, or transmucosally, or via osmotic pump, microcapsule, implant, or suspension. In certain embodiments of the invention (e.g., where the mu and/or delta opiate receptor antagonist has a relatively short compound half-life), it may be desirable to administer it using a time-release or slow-release formation, or transdermally (e.g., using a patch) in order to provide an administration half-life of sufficient length. When the mu and/or delta opiate receptor antagonist is administered transdermally or using a time-release or slow-release formulation, it is desirably released over a time period between two and twelve hours in duration; between two and six hours in duration; or between six and twelve hours in duration. In order to provide a high in vivo concentration of the ligand in a short amount of time, it may be desirable to administer the mu and/or delta opiate receptor antagonist using a rapidly absorbed loading dose. To provide a high in vivo concentration of the ligand quickly as well as a desirably long administration half-life, it may be desirable to use both a rapidly absorbed loading dose and transdermal administration or a time-release or slow-release formulation. A transdermal patch for naloxone, naltrexone and nalbuphine is disclosed in U.S. Pat. No. 4,573,995, which is hereby incorporated herein by reference in its entirety.

In certain embodiments of the invention, it may be desirable to administer both a specific mu and/or delta receptor antagonist and a non-specific mu and/or delta opiate receptor antagonist. The two types of antagonist may be administered substantially simultaneously or sequentially. Because the non-specific antagonists generally provide a greater counteradaptive effect than do specific mu or delta opiate antagonists, it is desirable to administer non-specific antagonists in the early stages of the method.

Because the body develops a tolerance to anti-opiates about eight days after first administration, it may be desirable to increase the dosage of the mu and/or delta opiate receptor antagonist with time. For example, it may be desirable to increase the dosage with a period of between a week and two weeks.

Desirably, an endorphin receptor agonist is not administered during the first time period associated with each administration. In certain embodiments of the invention, however, an endorphin receptor agonist is administered during one or more of the second time periods. Suitable but non-limiting examples of endorphin agonists include opiates such morphine, codeine, hydrocodone, fentanyl, and oxycodone. Morphine may be administered at dosages of 1-20-50 mg i.v. or 1-50 mg/hour continuous release via any suitable means such as transdermal, i.v., SQ, IM, or pump; Fentanyl may be administered at dosages of 0.1-0.5 mg gradual release over 8 hours via any suitable means such as transdermal, SQ, IM, or pump; Codeine may be administered at dosages of 10-100 mg p.o. every 4-6 hours; Hydrocodone may be administered at dosages of 5-25 mg p.o. every 4-6 hours; Oxycodone may be administered at dosages of 5-100 mg p.o. every 4 hours by any suitable means such as slow release transdermal, i.m., or SQ over 4-8 hours).

Enkephalins having an amino acid sequence of H-Tyr-Gly-Gly-Phe-Met-OH or H-Tyr-Gly-Gly-PheLeu-OH or any active analogues of these amino acid sequences with pharmacologically accepted carriers. Enkephalins may be administered at dosages of 1.0 μg/hr continuous release (transdermal, i.v., SQ, i.p. i.m. infusion pump).

Beta endorphin (a 31 amino acid peptide) or any and all active analogues, eg., beta-endorphin-(1-26), [D-Ala2]beta-endorphin or [Leu5]beta-endorphin with accepted pharmacologically accepted carriers. Beta endorphins may be administered at dosages of 1.0 μg/hr continuous release (e.g. transdermal, i.v., SQ, i.p. i.m. infusion pump).

Mu selective agonists such as Carfentanil which may be administered at a dosage of 1-25 μg/kg; [D-Ala2, NMe-Phe4, Gly-ol5] enkephalin and any active analogue with pharmacologically accepted carriers. The enkephalins may be administered at a suggested dosage of 1.0 μg/hr continuous release (e.g. i.v., i.m., SQ, pump, or transdermal).

Delta selective agonists such as DPDPE ([D-Pen2,D-Pen5]enkephalin); SB-235863; and SNC 80. DPDPE may be administered at a suggested dosage of 1.0-5.0 μg/hr continuous release (e.g., i.v., i.m., SQ, pump, or transdermal). SB-235863, ([8R-(4bS*,8aα,8aβ, 12bβ)]7,10-Dimethyl-1-methoxy-11-(2-methylpropyl)oxycarbonyl 5,6,7,8,12,12b-hexahydro-(9H)-4,8-methanobenzofuro[3,2-e]pyrrolo[2,3-g]isoquinoline Hydrochloride) may be administered at a dosage of 70 mg/kg p.o. See Paola Petrillo, et al. J. Pharmacology and Experimental Therapeutics, First published on Oct. 9, 2003; DOI: 10.1124/jpet.103.055590. SNC 80 may be administered at a dosage of 50-75 mg/kg slow release over several hours, transdermal, i.p. SQ, pump, etc.) See E J Bilsky, et al., Pharmacology and Experimental Therapeutics, Volume 273, Issue 1, pp. 359-366, 04/01/1995.

The endogenous endorphin system and its mu and/or delta opiate receptors are negatively linked to a wide variety of undesirable mental and neurological conditions. Examples of such conditions include pain, mood disorders, eating disorders, anxiety disorders, motivational problems, substance abuse disorders, insufficient motivation or performance, immune system-related conditions, wounds in need of healing, pain that is expected to occur in the future (e.g., due to a future operation or due to future physical exertion), chronic pain syndromes, acute pain, fibromyalgia, chronic fatigue syndrome, chronic back pain, chronic headaches, shingles, reflex sympathetic dystrophy, neuropathy, inflammatory pain, chronic cancer pain, major depressive disorders, post traumatic depression, temporary depressed mood, manic-depressive disorders, dysthymic disorders, generalized mood disorders, anhedonia, non-organic sexual dysfunction, overeating, obesity, anorexia, bulimia, a generalized anxiety state, panic disorders, Tourette's Syndrome, hysteria sleep disorders, breathing-related sleep disorders, lack of motivation due to learning or memory problems, abuse of a substance such as narcotics, alcohol, nicotine, stimulants, anxiolytics, CNS depressants, hallucinogens and marijuana, insufficient motivation for a desired mental or physical activity (e.g. physical training, athletics, studying or testing), immune-related condition such as infection, AIDS, or cancer, and wounds in need of healing. The up-regulation of the endogenous endorphin system desirably causes a therapeutic benefit with respect to the undesirable mental or neurological condition.

The endogenous endorphin system is implicated in pain because endorphins can bind to pain-mediating opiate receptors and decrease the synthesis of SP, a pain-inducing substance. The endogenous endorphin system has also been implicated in stress (U.S. Pat. Nos. 5,922,361 and 5,175,144), wound healing (Vinogradov V A, Spevak S E, et al, Bi and U.S. Pat. No. 5,395,398), substance abuse, eating disorders (Full & fulfilled: the science of eating to your soul's satisfaction, by Nan Allison; Carol Beck, Publisher: Nashville, T N: A & B Books,® 1998, ISBN: 0965911799), motivational problems (Tejedor-Real, P, et al, Eur J. Pharmacol. 1998 Jul. 31;354(1):1-7); immune response (Wybran, Fed Proc. 1985 January; 44(1 Pt 1):92-4, and U.S. Pat. No. 5,817,628) and cancer (Zagon, I S, et al., Cancer Lett, 1997; 112:167-175; U.S. Pat. Nos. 6,737,397; 6,136,780; and 4,801,614).

The endogenous endorphin system is also implicated in mood. Euphoria is the most recognizable emotional effect of opioids, which gives one an elevated feeling of well-being and care-free. Euphoria is modulated by endogenous endorphins. Endorphins are released with pleasurable experiences such as eating, exercise, winning an event, romantic encounters. It is thought that the endorphin release generates a feeling of well-being as a ‘reward’, which acts as a motivational mechanism in order to inspire an individual to fulfill nutritional and reproductive requirements. Another function of the endogenous endorphin system with respect to mood is to decrease anxiety, especially with regards to stress response. Rang H. P. (1995). Peptides as Mediators. In H. P. Rang & M. M. Dale, Pharmacology, Churchill Livingstone, N.Y.) demonstrates that endorphins are released at times of emotional stress, which acts to induce euphoria in order that anxiety is reduced.

Both endogenous endorphins and synthetic opiates may induce euphoria. The difference is that endogenous endorphins are rapidly degraded at their synapse and receptor sites, such that the effect is short term. With a short term effect there is no development of tolerance or dependency. Synthetic opiates, such as narcotics, have a much longer reactive time, thus they are associated with the development of dependency. Synthetic opiates have not been developed that have both, a strong analgesic effect and little or no potential for the development of dependency. Because endogenous endorphins have a similar euphoria-inducing capability as do opiates it is advantageous to use endogenous endorphins for inducing an elevated mood. However, because the administration of relatively large and prolonged doses of synthetic endorphins may be associated with the development of tolerance and dependency, they are not desirable long-term treatment agents.

Both mu and delta opiate receptors are involved to some degree with mood. Mu receptors primarily mediate pain perception, but also induce euphoria when these receptors are bound by endorphin/opiate compounds. The role of delta receptors in pain modulation is not clear, whereas they are likely more closely related to euphoria. Delta receptor agonists demonstrate anti-depressant activity in rats in the forced swim assay. Furthermore, evidence from animal studies demonstrates that delta-opioid receptors are involved in motivational activities. Their preferential involvement is through enkephalin-controlled behavior. Broom, et al. (Jpn J. Pharmacol. 2002 September; 90(1): 1-6) demonstrate that the delta opiate receptor plays a significant role in depression.

Methods of the present invention using mu and/or delta receptor antagonists as ligands may be used to address undesirable mental or neurological conditions in patients. For example the methods of the present embodiment of the invention may be used to address any of the above-listed conditions. The methods according to the present embodiment of the invention may also be used as an adjunct treatment for cancer.

Methods of the present invention using mu and/or delta receptor antagonists may be used to address pain that is anticipated to occur in the future. For example, if a patient is scheduled for elective surgery in, e.g., one month then the method of the present invention can be practiced with a mu and/or delta opiate receptor, using high night-time dosing for the intervening pre-operative period of time. After surgery the patient will have an enhanced response to pain due to the up-regulated endogenous endorphin system. In addition, the patient will require lower overall doses of narcotic pain medications post-operatively due to enhanced sensitivity of mu and/or delta opiate receptors. The method would likely best interrupted immediately after surgery so that post-operative pain would not increase due to the direct effects of receptor antagonism. It could be restarted in a few days or so, once the pain had subsided, in order to maintain the counteradaptive response.

In an example of a pre-operative treatment according to the present invention, a 49 year old male is scheduled for reconstructive surgery on his knee in 6 weeks. He is begun on a naloxone patch, 200 mg, which is formulated to be rapidly absorbed over 6-8 hours as described above, on a nightly basis. To reduce the anxiety that this induces he is given an anxiolytic agent, diazepam (1-5 mg) at night along with the naloxone patch. After 2 weeks of this dose, the naloxone is increased to 400 mg on a nightly basis. The anxiolytic agent is used if needed. After yet an additional 2 weeks the naloxone is increased to 600-800 mg on a nightly basis. On the night of surgery and for several nights in the peri-operative period no naloxone is given. The patient is given only standard post-operative pain medications such as morphine and codeine. The doses of these substances are significantly reduced compared to the average individual undergoing this type of surgery, due to the up regulation of this patient's endorphin system. In an alternative method, after the first 2 weeks of naloxone treatment, the same patient is given a specific mu receptor antagonist along with the increasing dose of naloxone, in order to enhance the up regulation of pain-modulating mu receptors.

The methods of the invention may be used with mu and/or delta antagonists to elevate a patient's mood in the treatment of depression and related conditions. At first, non-specific opiate receptor antagonists (e.g., naloxone) may be administered to induce a counteradaptive response. Later in the treatment, it may be desirable to administer a specific delta opiate receptor antagonist because delta opiate receptors are strongly linked to mood. Of course, mu opiate receptor antagonists could be used, especially when chronic pain is associated with the depressed mood. When treating an already-depressed patient, the skilled artisan will closely monitor the patient for ill effects due to any acute worsening of mood due to antagonist-receptor binding.

In an example of a method of treating a depressed patient using the methods of the present invention, a 35 year old male with a diagnosis of clinical depression has had poor response and side effects with conventional antidepressant agents. He is especially consulted on the potential for temporary worsening of the depressed state, including suicidal tendencies. In-patient treatment in a hospital or appropriate mental institution is considered at the onset of therapy for higher risk potentially suicidal patients. After this is worked out, he is started on counteradaptation therapy protocol with the non-specific opiate antagonist naloxone. A transmucosal naloxone formulation is started prior to going to sleep, using a loading dose of 20 mg. A 30 mg transdermal dose, formulated to be absorbed over 6 hours, is applied at the same time. This 50 mg per 8 hour dose is given for two weeks. At two weeks the transmucosal dose is increased to 50 mg. The 6 hour transdermal dose is 50 mg, for a total of 100 mg. This dose is given for one month. Now, at 6 weeks after treatment had begun, the loading dose is 100 mg transmucosal and 100 mg transdermal over 6 hours. After another 4-6 weeks this is increased to 250 mg loading dose and 250 mg over 6 hours for a total 500 mg. After another on to two months this is increased to 500 mg loading dose and 500 mg over the next 6 hours. After another one or two or three months this is increased to a 1000 mg loading dose and a 1000 mg 6 hour transdermal dose. The maximal can stay for a long period of time at this 2000 mg total dose. Or it can continue to increase to 2,500, or 3,000 or 4,000 mg over the ensuing year or more. The maximal dose comes to a plateau once there is a good clinical response or once the side effects become too great or if there is an elevation of liver function enzymes on a blood test. The maximum tolerable dose is then given for an extended period of time for maintenance therapy. If and when therapy is stopped the patient is carefully monitored for any signs of recurrence of the mood disorder.

An option for the above-described patient is to add a delta opiate receptor antagonist, along with the naloxone after the first 6 weeks to 3 months of treatment. The naloxone dose may continue to be increased or it may level off earlier when combined with the delta antagonist. A non-peptide delta opiate receptor antagonist, such as naltrindole, natriben, or one of the agents discussed above, could be used. A peptide delta antagonist, such as ICI-154,129 or ICI-174,864 peptide, could also be used. The starting dose for naltrindole is larger than that for naloxone. It may be as high as 10 mg/kg/administration. Naltrindole may be given as a transdermal compound or using any other effective formulation.

The main consideration is the dosing for people with significant depression who may be at risk for suicide if the initial doses are too large. In a desirable embodiment of the invention, people with clinical depression, because they are suicidal risks, should either not be treated or treated at an in-patient hospital or appropriate institution in order to better monitor the patient. These patients are dosed at relatively lower doses at the beginning of treatment and that the increase in dose is done at a slower rate. Thus, for the depressed patients treatment may need to be started with a loading of only 10 mg of naloxone, with 10 or 20 mg to be absorbed over the ensuing 6 hours, for a total starting dose of 30 mg. Similarly, the increase in dose after 2 weeks is more gradual than for the example above. At 2 weeks one would give 20 mg as a loading dose and 20-40 mg over the ensuing 6 hours. This gradual increase is continued for as many months as is needed to obtain a maximal clinical response.

### The Dynorphin System

According to another embodiment of the invention, the neurotransmitter system is the dynorphin system, which includes dynorphins as neurotransmitters. Dynorphins are a class of endorphin compounds that bind preferentially to kappa receptors. Dynorphins generally have the opposite effect from the endorphins; their binding to kappa receptors is associated with a worsening of mood.

When the neurotransmitter system is the dynorphin system, the type of receptor is kappa receptors, which are positively linked to undesirable mental and neurological conditions. Kappa receptors are associated primarily with dysphoria when stimulated. The ligand is a kappa receptor agonist, and the counteradaptation causes a down-regulation of the dynorphin system. The counteradaptation may be, for example, a decrease in the biosynthesis or release of dynorphins at receptor terminals and/or by the pituitary gland; a decrease in the number of the receptors and/or dynorphin binding sites on the receptors; a decrease in the sensitivity of the receptors to binding by mu and/or delta receptor agonists and/or dynorphins; or a combination thereof. The counteradaptation may also up-regulate D2 (dopamine) receptors, which are negatively linked to depression.

A variety of kappa receptor agonists may be used in the present invention. For example, the kappa receptor agonist may be a peptide-based agonist, such as dynorphin [Dynorphin [A 1-17], H-TYR-GLY-GLY-PHE-LEU-ARG-ARG-ILE8-ARG-PRO-LYS-LEU-LYS-TRP-ASP-ASN-GLN-OH] and all active peptide fragments and analogues thereof or a pharmaceutically acceptable salt, or carrier thereof. For example, the kappa receptor agonist may be the active C-terminal fragment of dynorphin A(1-8), or a pharmaceutically accepted salt or carrier thereof.

The kappa receptor agonist may also be non-peptidic. For example, the kappa receptor agonist may be a nonbenzomorphan; enadoline; PD117302; CAM569; PD123497; GR 89,696; U69,593; TRK-820; trans-3,4-dichloro-N-methyl-N-[1-(1-pyrrolidinyl)cyclohexyl]benzene-acetamide; asimadoline (EMD-61753); benzeneacetamide; thiomorpholine; piperidine; benzo[b]thiophene-4-acetamide; trans-(+/−)-(PD-117302); 4-benzofuranacetamide (PD-129190); 2,6-methano-3-bezazocin-8-ol (MR-1268); morphinan-3-ol (KT-90); GR-45809; 1-piperazinecarboxylic acid (GR-89696); GR-103545; piperzaine; GR-94839; xorphanl; benzeneacetamide (RU-49679); fedotozine; benzeneacetamide (DuP-747); HN-11608; apadoline (RP-60180); spiradoline mesylate; benzeneacetamide trans-U-50488 methane sulfate; 3FLB; FE200665; FE200666; an analogue of MPCB-GRR1 or MPCB-RR1; benzomorphan kappa opioids, such as bremazocine and ethylketocyclazocine; or a pharmaceutically-accepted salt or carrier thereof.

The kappa receptor agonist may be U50,488 (trans-3,4-dichloro-N-[2-(1-pyrrolidinyl) cyclohexyl]benzeacetamide) and spiradoline (U62,066E). Enadoline and PD117302 {Enadoline [(5R)-5α, 7x, 8β-N-methyl-N-[7-(1-pyrrolidinyl)-1-oxzspiro[4,51 dec-8-yl]-4-benzofuranacetamide monohydrochloride], PD117302 [(±)-trans-N-methyl-N-[2-(1-pyrrolidinyl)-cyclohexyl]benzo[b]thiophene-4-acetamide monohydrochloride]and their respective (+)-isomers (CAM569 and PD123497) (Parke-Davis Research Unit, Cambridge, UK)} are highly selective arylacetamide kappa opioids. GR89,696 (4-[(3,4-dichloro-phenyl) acetyl]-3-(1-pyrrolidinylmethyl)-1-piperazinecarboxylic acid methyl ester fumarate) is a prototypical arylacetamide developed from the structure of U50,488H. It has high efficacy as a K₁ agonist. U69,593 [(5 alpha, 7 alpha, 8 beta)-(+)-N-methyl-N-(7-(1-pyrrolidinyl)-1-oxaspiro(4,5)dec-8-yl)benzeneacetamide]is also a kappa agonist with K₁ selectivity. TRK-820 ((−)-17-cyclopropylmethyl-3,14b-dihydroxy-4,5a-epoxy-6b-[N-methyl-trans-3-(3-furyl) acrylamide]morphinan hydrochloride) (Toray Industries, Inc. Japan) is a potent kappa agonist with pharmacological properties different from those produced by K₁ receptor agonists. Tifluadom is a benzodiazepine kappa agonist (Sandoz, Inc., Princeton, N.J.). U.S. Pat. No. 4,758,562 also describes the kappa agonist: trans-3,4-dichloro-N-methyl-N-[1-(1-pyrrolidinyl)cyclohexyl]benzeneacetamide.

Kappa receptor agonists are also described in U.S. Pat. Nos. 5,051,428; 5,965,701; 6,146,835; 6,191,126; 6,624,313; 6,174,891; 6,316,461; 6,440,987; 4,758,562; 6,583,151, each of which is incorporated herein by reference in its entirety.

The initial dosage of the kappa receptor agonist is desirably high enough to induce a counteradaptive effect, but not so high as to cause the patient intolerable direct effects. For example, the initial dosage of the kappa receptor agonist may be equivalent to between 0.0005 and 0.05 mg/kg/administration of dynorphin; between 5 and 700 mg/administration of enadoline; between 1 and 500 μg/administration of FE 20665; between 0.5 and 100 μg/administration; between 0.01 and 1 mg/kg/administration of U69,593; between 0.05 and 5 mg/kg/administration of TRK 820; between 0.01 and 1 mg/kg/administration U 50 488 or between 0.01 and 1 mg/kg/administration of PD 117302. Desirably, the initial dosage of the kappa receptor agonist is equivalent to between 0.005 and 0.02 mg/kg/administration of dynorphin; between 100 and 500 mg/administration of enadoline; between 3 and 100 μg/administration of FE 20665; between 1 and 80 μg/administration of FE 20666; between 0.1 and 0.7 mg/kg/administration of U69,593; between 0.5 and 3 mg/kg/administration of TRK 820; between 0.5 and 7 mg/kg/administration U 50 488 or between 0.1 and 0.7 mg/kg/administration of PD 117302.

In another embodiment of the invention, the kappa receptor agonist is Salvinorin A. Salvinorin A is a neoclerodane diterpene compound, which is a very powerful hallucinogen that has recently been found to have kappa agonist activity. It represents the only known non-nitrogenous kappa agonist compound. It is the main active ingredient of the plant S. divinorum (Diviner's sage), a rare member of the mint family. It has been used for many centuries by the Mazatec people of Oaxaca, Mexico in traditional spiritual practices. The initial dose of Salvinorin A is desirably between 5 and 50 ug/administration, and the maximum dose is desirably 5000 ug/administration. The Salvornin A may be administered transmucosally, or as a slow-release formulation, desirably over a period between two and six hours in duration.

In certain embodiments of the invention, it may be desirable to administer both a peptidic kappa receptor agonist and a non-peptidic kappa receptor agonist. The two types of agonist may be administered substantially simultaneously, or sequentially.

Peptidic kappa receptor agonists may be administered, for example, intravenously, transdermally, or transmucosally, as described above with respect to other peptidic ligands. As described above with respect to naloxone, it may be desirable to use transmucosal administration (to achieve a high level of ligand-receptor binding quickly) along with transdermal administration (to provide extended ligand-receptor binding).

Because the body develops a tolerance to anti-opiates about eight days after first administration, it may be desirable to increase the dosage of the kappa receptor agonist with time. For example, it may be desirable to increase the dosage with a period of between a week and two weeks.

In an example of a method of the present invention using Salvinorum A, the initial dose of Salvinorum A is low in order to decrease potential side effects. A dose between 5 μg-50 μg is the starting dose. After 2-4 weeks this is increased by a certain percent. The increase could be as small as 5-10% or 50-100% or more. Generally, a doubling of the initial dose is recommended. Thus, after 2-4 weeks the individual is given 20-100 μg of Salvinorum A. This increase in dose is continued every two, four, six or eight weeks. It may also continue to increase on a quarterly, semiannual or annual basis. Doses of 200 μg may produce increasing dysphoric effects. This occurs with acute administration. With chronic gradual increase in dose the side effects would be gradually muted. With chronic gradual increase in dosing the maximum dose of Salvinorum A is 1000 μg to 5000 μg or more.

In an example of the method of the present invention using a dynorphin analogue, a rectal suppository (transmucosal) formulation is used. The initial dose is high enough in order to induce a counteradaptive response, but low enough to minimize dysphoric effects of agonist-receptor binding. There is a two-part construct of the suppository. The outer covering is rapidly dissolved and allows for an initial rapid absorption of the kappa receptor agonist compound. The second layer is gradually broken down in order to slowly release additional kappa receptor agonist, which is gradually absorbed. This results in a continuous, slow-release absorption of the peptide kappa receptor agonist compound. It is designed to last for 6-8 hours of gradual absorption such that there is 6-8 hours of kappa receptor binding, at which time the counteradaptive response is induced. This rectal suppository is given on a daily (nightly) basis. After 2-4 weeks the dose is doubled. This dose is then given for an additional 2-4-6-8 weeks. The dose is intermittently increased until the development of side effects prevents a further increase. As the dose is increased the time interval for increasing the dose is lengthened, such that several months may pass before increasing the dose. In addition, once higher doses are used the increase is less dramatic, such that only 5-10% increases are given, rather than the initial doubling of the dose.

Enadoline is a non-peptidic kappa receptor agonist. It has pharmaceutical activity when taken as an oral dose at 1-10 mg/kg. In an example of a method of the present invention using enadoline, an initial dose of 100-200 mg is administered daily just prior to the patient's going to bed. After 2-4 weeks the dose is increase to 200-500 mg. After another 2-4 weeks the dose is increased to 500-1000 mg. After another two, four, eight weeks or more, it is increased to 1500-2000 mg. The dose is increased as long as side effects do not become uncontrollable.

Desirably, a kappa receptor antagonist is not administered during the first time period associated with each administration. In certain embodiments of the invention, however, a kappa receptor antagonist is administered during one or more of the second time periods. Representative kappa receptor antagonists include the compounds described in U.S. Pat. Nos. 5,025,018; 5,922,887; and 6,284,769. For the compounds described in 5,025,018, a suitable dosage includes 0.1 to 10 mg/administration per day; for U.S. Pat. No. 6,284,769, suitable dosages include 0.1 to 500 mg/administration.

The dynorphin neurotransmitter system and its kappa receptors are positively linked to a wide variety of undesirable mental and neurological conditions. Examples of such conditions include pain, mood disorders, eating disorders, anxiety disorders, motivational problem, substance abuse disorders, insufficient motivation or performance, pain that is expected to occur in the future (e.g., due to a future operation or future physical exertion), chronic pain syndromes, acute pain, fibromyalgia, chronic fatigue syndrome, chronic back pain, chronic headaches, shingles, reflex sympathetic dystrophy, neuropathy, inflammatory pain, chronic cancer pain, major depressive disorders, post traumatic depression, temporary depressed mood, manic-depressive disorders, dysthymic disorders, generalized mood disorders, anhedonia, non-organic sexual dysfunction, overeating, obesity, anorexia, bulimia, generalized anxiety state, panic disorders, Tourette's Syndrome, hysteria sleep disorders, breathing-related sleep disorders, lack of motivation due to learning or memory problems, abuse of substances such as narcotics, alcohol, nicotine, stimulants, anxiolytics, CNS depressants, hallucinogens and marijuana, and insufficient motivation for a desired mental or physical activity such as physical training, athletics, studying or testing. The down-regulation of the dynorphin system desirably causes a therapeutic benefit with respect to the undesirable mental or neurological condition.

### The Serotonin System

According to another embodiment of the invention, the neurotransmitter system is the serotonin system which includes serotonin as a neurotransmitter. Serotonin is a monoamine neurotransmitter. Low serotonin levels are associated with depression. The counteradaptation causes an up-regulation of the serotonin system.

Numerous serotonin receptors (at least 14) have been identified. The greatest concentration of serotonin (90%) are located in the gastrointestinal tract. Most of the remainder of the body's serotonin is found in platelets and the central nervous system (CNS). The effects of serotonin are noted in the cardiovascular system, the respiratory system and the intestines. Vasoconstriction is a typical response to serotonin.

The function of serotonin is exerted upon its interaction with specific receptors. Several serotonin receptors have been cloned and are identified as 5HT₁, 5HT₂, 5HT₃, 5HT₄, 5HT₅, 5HT₆, and 5HT₇. Within the 5HT₁ group there are subtypes 5HT_{1A}, 5HT_{1B}, 5HT_{1D}, 5HT_{1E}, and 5HT_{1F}. There are three 5HT₂ subtypes, 5HT_{2A}, 5HT_{2B}, and 5HT_{2C} as well as two 5HT₅ subtypes, 5HT₅ₐ and 5HT_{5B}. Most of these receptors are coupled to G-proteins that affect the activities of either adenylate cyclase or phospholipase Cg. The 5HT₃ class of receptors are ion channels

Some serotonin receptors are presynaptic and others postsynaptic. The 5HT_{2A} receptors mediate platelet aggregation and smooth muscle contraction. The 5HT_{2C} receptors are suspected in control of food intake as mice lacking this gene become obese from increased food intake and are also subject to fatal seizures. The 5HT₃ receptors are present in the gastrointestinal tract and are related to vomiting. Also present in the gastrointestinal tract are 5HT₄ receptors where they function in secretion and peristalsis. The 5HT₆ and 5HT₇ receptors are distributed throughout the limbic system of the brain and the 5HT₆ receptors have high affinity for antidepressant drugs.

The most common serotonin receptors that are associated with mood and depression are the 1^{st} and 2^{nd} ones, most especially the 5HT_{1A} receptors.

When a serotonin neuron is stimulated to fire, serotonin is released into the synapse. Some serotonin molecules cross the synapse and bind to the post-synaptic receptor, which then causes firing of the post-synaptic serotonin neuron. Binding of serotonin to the post-synaptic serotonin neuron causes its activation, which leads to a series of neural events that is associated with a generally good mood.

When serotonin is released into the synaptic cleft only a portion of the serotonin actually binds to post-synaptic receptors. The majority of serotonin molecules are removed from the synapse by a reuptake mechanism. Some of this serotonin is degraded by monoamine oxidases, enzymes that degrade both serotonin and norepinephrine.

The third target of serotonin molecules are the pre-synaptic auto-receptors. The pre-synaptic autoreceptors are inhibitory receptors. The pre-synaptic autoreceptors act in a feedback inhibition loop that functions as a control mechanism for neurotransmitter release. A feedback inhibition loop is a common manner by which the body controls the activation of neurons. When they are bound by sertonin, or an agonist, they inhibit the further release of sertonin into the synapse. Pre-synaptic autoreceptors are termed 5HT_{1A} and 5HT_{1B} pre-synaptic autoreceptors. 5HT_{1A} autoreceptors inhibit the tonic release of serotonin. 5HT_{1B} autoreceptors are thought to inhibit the evoked release and synthesis of serotonin.

When the neurotransmitter system is the serotonin system, the type of receptor may be, for example, serotonin pre-synaptic autoreceptors such as 5HT_{1A} autoreceptors or 5HT_{1B} autoreceptors. In such cases, the ligand is a serotonin pre-synaptic autoreceptor agonist, and the undesirable mental or neurological condition is positively linked to the receptors. The counteradaptation may be, for example, an increase in the biosynthesis and/or release of serotonin at the synaptic cleft; a decrease in the reuptake of serotonin; a decrease in the number of serotonin pre-synaptic autoreceptors; a decrease in the sensitivity of the serotonin pre-synaptic autoreceptors to serotonin and/or serotonin pre-synaptic autoreceptor agonists; an increase in the number of serotonin post-synaptic receptors; an increase in the sensitivity of the serotonin post-synaptic receptors to serotonin or serotonin post-synaptic receptor agonists; or a combination thereof.

A variety of serotonin pre-synanptic autoreceptor agonists may be used in the methods of the present invention. For example, the serotonin pre-synaptic autoreceptor agonist may be EMD-68843, buspirone, gepirone, ipsapirone, tandospirone, Lesopitron, zalospirone, MDL-73005EF, or BP-554.

The initial dosage of the serotonin pre-synaptic autoreceptor agonist is desirably high enough to induce a counteradaptive effect, but not so high as to cause the patient intolerable direct effects. For example, the initial dosage of the serotonin pre-synaptic autoreceptor agonist may be equivalent to between 1 and 400 mg/administration of EMD-68843, between 1 and 500 mg/administration buspirone, between 1 and 500 mg/administration lesopitron, between 1 and 500 mg/administration gepirone, between 5 and 500 mg tandospirone, or between 1 and 200 mg zalospirone. Desirably, the initial dosage of the serotonin pre-synaptic autoreceptor agonist is equivalent to between 10 and 100 mg/administration of EMD-68843, between 10 and 100 mg/administration buspirone, between 10 and 200 mg/administration lesopitron, between 10 and 100 mg/administration gepirone, between 20 and 200 mg tandospirone, or between 10 and 100 mg zalospirone.

Desirably, a serotonin pre-synaptic autoreceptor antagonist is not administered during the first time period associated with each administration. In certain embodiments of the invention, however, a serotonin pre-synaptic autoreceptor antagonist is administered during one or more of the second time periods. Representative serotonin pre-synaptic autoreceptor 5HT1A agonists and antagonists include Elazonan, AR-A2 (AstraZeneca, London, UK); AZD-1134 [AstraZeneca, London, UK); Pindolol, as well as compounds described in U.S. Pat. Nos. 6,462,048; 6,451,803; 6,627,627; 6,602,874; 6,277,852; and 6,166,020, incorporated by reference in their entirety.

In another embodiment of the invention, the type of receptor is serotonin post-synaptic receptors, such as are 5HT₁ receptors; 5HT₂ receptors; 5HT₃ receptors; 5HT₄ receptors; 5HT₅ receptors; 5HT₆ receptors; 5HT₇ receptors; or receptors of a subtype thereof. The ligand is a serotonin post-synaptic receptor antagonist, and the undesirable mental or neurological condition is negatively linked with the receptors. The counteradaptation may be an increase in the biosynthesis and/or release of serotonin at the synaptic cleft; a decrease in the reuptake of serotonin; an increase in the number of serotonin post-synaptic receptors; an increase in the sensitivity of the serotonin post-synaptic receptors to serotonin and/or serotonin post-synaptic receptor agonists; a decrease in the number of serotonin pre-synaptic autoreceptors; a decrease in the sensitivity of the serotonin pre-synaptic autoreceptors to serotonin and/or serotonin pre-synaptic autoreceptor agonists; or a combination thereof.

A variety of compounds may be used as the serotonin post-synaptic receptor antagonists in the methods of the present invention. For example, the serotonin post-synaptic receptor antagonists may be (S)-WAY-100135, WAY-100635, buspirone, gepirone, ipsapirone, tandospirone, Lesopitron, zalospirone, MDL-73005EF, or BP-554. If desired, an SSRI maybe administered either simultaneously or sequentially with the aforementioned serotonin modulating agents. This is advantageous as both SSR1 and agonist pre-synaptic counteradaptive therapy result in a down regulation of the pre-synaptic receptors. The SSR1 effect is thus magnified by such a counteradaptive effect. Second, any down regulation of the post synaptic serotonin receptors that may occur with SSR1 therapy is counterbalanced by post synaptic antagonist counteradaptive therapy.

The initial dosage of the serotonin post-synaptic antagonist is desirably high enough to induce a counteradaptive effect, but not so high as to cause the patient intolerable direct effects. For example, the initial dosage of the serotonin post-synaptic receptor antagonist is equivalent to between about 0.01 and 5 mg/kg/administration of WAY-100635. Desirably, the initial dosage of the serotonin post-synaptic receptor antagonist is equivalent to between about 0.025 and 1 mg/kg/administration of WAY-100635.

The serotonin post-synaptic receptor antagonist may be administered in combination with a serotonin pre-synaptic autoreceptor agonist, such as those described above. Further, when conventional anti-depressant agents that bind at the serotonin post-synaptic receptors are given in combination with a serotonin pre-synaptic autoreceptor agonist, its efficacy can be greatly increased because the serotonin post-synaptic receptors have increased in number and/sensitivity through the counteradaptation.

In certain desirable embodiments of the invention, the serotonin post-synaptic antagonist itself is also a serotonin pre-synaptic autoreceptor agonist. It may also be desirable to administer a norepinephrine pre-synaptic alpha-2 adrenergic receptor agonist and/or a norepinephrine post-synaptic adrenergic receptor antagonist (as described below) in combination with the serotonin post-synaptic antagonist or serotonin pre-synaptic autoreceptor agonist.

Desirably, a serotonin post-synaptic receptor agonist is not administered during the first time period associated with each administration. In certain embodiments of the invention, however, a serotonin post-synaptic receptor agonist is administered during one or more of the second time periods. Representative serotonin post-synaptic receptor agonists include BIMT 17 (1-[2-[4-(3-trifluoromethyl phenyl) piperazin-1-yl]ethyl] benzimidazol-[1H]-2-one), dose: 1-10 mg/kg (i.v. or transdermal, SQ, etc.). See Borsini, F, et al., Archives of Pharmacology, 352(3); September, 1995: 283-290.]A suitable dosage range includes 1 to 10 mg/kg/administration of BIMT 17 (via iv, transdermal, or SQ).

Serotonin post-synaptic receptors are negatively linked, and serotonin pre-synaptic autoreceptors are positively linked to a wide variety of undesirable mental and neurological conditions. Examples of such conditions include pain, mood disorders, eating disorders, anxiety disorders, obsessive-compulsive disorders, motivational problem, substance abuse disorders, insufficient motivation or performance, pain that is expected to occur in the future (e.g., due to a future operation or future physical exertion), chronic pain syndromes, acute pain, fibromyalgia, chronic fatigue syndrome, chronic back pain, chronic headaches, shingles, reflex sympathetic dystrophy, neuropathy, inflammatory pain, chronic cancer pain, major depressive disorders, post traumatic depression, temporary depressed mood, manic-depressive disorders, dysthymic disorders, generalized mood disorders, anhedonia, non-organic sexual dysfunction, overeating, obesity, anorexia, bulimia, generalized anxiety state, panic disorders, Tourette's Syndrome, hysteria sleep disorders, breathing-related sleep disorders, lack of motivation due to learning or memory problems, abuse of substances such as narcotics, alcohol, nicotine, stimulants, anxiolytics, CNS depressants, hallucinogens and marijuana, and insufficient motivation for a desired mental or physical activity such as physical training, athletics, studying or testing. The up-regulation of the serotonin system desirably causes a therapeutic benefit with respect to the undesirable mental or neurological condition.

### The Norepinephrine System

In another embodiment of the invention, the neurotransmitter system is the norepinephrine system which includes norepinephrine as a neurotransmitter, and the counteradaptation causes an up-regulation of the norepinephine system.

Norepinephrine is a catecholamine that, along with epinephrine, acts as a neurotransmitter in the central nervous system. There are two types of adrenoreceptors, alpha and beta. There are in addition, at least ten different subtypes of adrenoreceptors. Norepinephrine generally is more potent at sites where sympathetic neurotransmission is excitatory and is mediated through alpha receptors. Alpha receptors have two main subclasses, alpha1 and alpha2.

Norepinephrine acts a neuromodulator in the central nervous system. The central nervous system actions of NE are most notable when it modulates excitatory or inhibitory inputs, rather than its effects on the activity of post-synaptic targets, in the absence of other inputs. Norepinephrine transmission and control is similar to that for serotonin. A reuptake mechanism is present that removes the majority of norepinephrine after its release into the noradrenergic synapse. There are pre-synaptic inhibitory autoreceptors known as alpha-2 adrenergic receptors.

When the neurotransmitter system is the norepinephrine system, the type of receptor may be, for example, norepinephrine pre-synaptic alpha-2 adrenergic receptors. In such cases, the ligand is a norepinephrine pre-synaptic alpha-2 adrenergic receptor agonist, and the undesirable mental or neurological condition is positively linked to the receptors. The counteradaptation may be an increase in the biosynthesis and/or release of norepinephrine at the synaptic cleft; a decrease in reuptake of norepinephrine; a decrease in the number of norepinephrine pre-synaptic alpha-2 adrenergic receptors; a decrease in the sensitivity of the norepinephrine pre-synaptic alpha-2 adrenergic receptors to norepinephrine and/or norepinephrine pre-synaptic alpha-2 adrenergic receptor agonists; an increase in the number of norepinephrine post-synaptic adrenergic receptors; an increase in the sensitivity of the norepinephrine post-synaptic adrenergic receptors to norepinephrine and/or norepinephrine post-synaptic adrenergic receptor agonists; or a combination thereof.

A variety of compounds may be used as the norepinephrine pre-synaptic alpha-2 adrenergic receptor agonists in the methods of the present invention. For example, the norepinephrine pre-synaptic alpha-2 adrenergic receptor agonist may be clonidine, guanfacine, lofexidine, detomidine, dexmedetomidine, mivazerol, or alpha-methylnoradreniline.

The initial dosage of the norepinephrine pre-synaptic alpha-2 adrenergic receptor agonist is desirably high enough to induce a counteradaptive effect, but not so high as to cause the patient intolerable direct effects. For example, the initial dosage may be equivalent to between 0.1 and 10 μg/kg/administration of clonidine, between 0.01 and 10 mg/administration guanfacine, between 0.01 and 1 mg/administration lofexidine, between 1 and 100 μg/kg/administration detomidine, between 0.05 and 5 μg/kg/administration dexmedetomidine, between 0.05 and 10 μg/kg/administration mivazerol, or between 5 and 500 ng/kg/administration of alpha-methylnoradreniline. Desirably, the initial dosage is equivalent to between 0.1 and 0.5 mg/administration of clonidine, between 0.1 and 5 mg/administration guanfacine, between 0.05 and 0.5 mg/administration lofexidine, between 10 and 80 μg/kg/administration detomidine, between 0.1 and 3 μg/kg/administration dexmedetomidine, between 0.5 and 5 μg/kg/administration of mivazerol, or between 10 and 100 ng/kg/administration of alpha-methylnoradreniline.

Desirably, a norepinephrine pre-synaptic alpha-2 adrenergic receptor antagonist is not administered during the first time period associated with each administration. In certain embodiments of the invention, however, an norepinephrine pre-synaptic alpha-2 adrenergic receptor antagonist is administered during one or more of the second time periods. A suitable non-limiting example of a pre and postsynaptic A2AR antagonist includes mirtazapine.

According to another embodiment of the invention, the type of receptor is norepinephrine post-synaptic adrenergic receptors, such as alpha receptors, beta receptors, or receptors of a subtype thereof. In such cases, the ligand is a norepinephrine post-synaptic adrenergic receptor antagonist, and the undesirable mental or neurological condition is negatively linked to the norepinephrine post-synaptic adrenergic receptors. The counteradaptation may be an increase in the biosynthesis or release of norepinephrine at the synaptic cleft; a decrease in the reuptake of norepinephrine; an increase in the number of norepinephrine post-synaptic adrenergic receptors; an increase in the sensitivity of the norepinephrine post-synaptic adrenergic receptors to norepinephrine and/or norepinephrine post-synaptic adrenergic receptor agonists; a decrease in the number of norepinephrine pre-synaptic alpha-2 adrenergic receptors; a decrease in the sensitivity of the norepinephrine pre-synaptic alpha-2 adrenergic receptors to norepinephrine and/or norepinephrine pre-synaptic alpha-2 adrenergic receptor agonists; or a combination thereof.

A variety of compounds may be used as the norepinephrine post-synaptic adrenergic receptor antagonists in the methods of the present invention. For example, the norepinephrine post-synaptic adrenergic receptor antagonist may be idazoxan, SKF 104078, or SKF 104856. The initial dosage of the norepinephrine post-synaptic adrenergic receptor antagonist is desirably high enough to induce a counteradaptive effect, but not so high as to cause the patient intolerable direct effects. For example, the initial dosage may be equivalent to between 0.5 and 100 mg/administration of idazoxan. Desirably, the initial dosage is equivalent to between 5 and 50 mg/administration of idazoxan.

Desirably, a norepinephrine post-synaptic adrenergic receptor agonist is not administered during the first time period associated with each administration. In certain embodiments of the invention, however, a norepinephrine post-synaptic adrenergic receptor agonist is administered during one or more of the second time periods.

The norepinephrine post-synaptic adrenergic receptor antagonist may be administered in combination with a norepinephrine pre-synaptic alpha-2 adrenergic receptor agonist, such as those described above. Further, when conventional anti-depressant agents that bind at the norepinephrine post-synaptic adrenergic receptors are given in combination with a norepinephrine pre-synaptic alpha-2 adrenergic receptor agonist, its efficacy can be greatly increased because the norepinephrine post-synaptic adrenergic receptors have increased in number and/sensitivity through the counteradaptation.

In certain desirable embodiments of the invention, the norepinephrine post-synaptic adrenergic receptor antagonist itself is also an norepinephrine pre-synaptic alpha-2 adrenergic receptor agonist. It may also be desirable to administer a serotonin post-synaptic antagonist and/or a serotonin pre-synaptic autoreceptor agonist (as described above) in combination with the norepinephrine pre-synaptic alpha-2 adrenergic receptor agonist or norepinephrine post-synaptic adrenergic receptor antagonist.

Norepinephrine post-synaptic adrenergic receptors are negatively linked, and norepinephrine pre-synaptic alpha-2 adrenergic receptors are positively linked to a wide variety of undesirable mental and neurological conditions. Examples of such conditions include pain, mood disorders, eating disorders, anxiety disorders, obsessive-compulsive disorders, motivational problem, substance abuse disorders, insufficient motivation or performance, pain that is expected to occur in the future (e.g., due to a future operation or future physical exertion), chronic pain syndromes, acute pain, fibromyalgia, chronic fatigue syndrome, chronic back pain, chronic headaches, shingles, reflex sympathetic dystrophy, neuropathy, inflammatory pain, chronic cancer pain, major depressive disorders, post traumatic depression, temporary depressed mood, manic-depressive disorders, dysthymic disorders, generalized mood disorders, anhedonia, non-organic sexual dysfunction, overeating, obesity, anorexia, bulimia, generalized anxiety state, panic disorders, Tourette's Syndrome, hysteria sleep disorders, breathing-related sleep disorders, lack of motivation due to learning or memory problems, abuse of substances such as narcotics, alcohol, nicotine, stimulants, anxiolytics, CNS depressants, hallucinogens and marijuana, and insufficient motivation for a desired mental or physical activity such as physical training, athletics, studying or testing. The up-regulation of the norepinephrine system desirably causes a therapeutic benefit with respect to the undesirable mental or neurological condition.

As the skilled artisan will appreciate, different ligands for different receptors can be administered in combination either sequentially or simultaneously. For example, repeated administration of a mu and/or delta opiate antagonist can be followed by (or performed simultaneously with) repeated administration of an SP receptor antagonist. If desired, an NRI maybe administered either simultaneously or sequentially with the aforementioned NE modulating agents. Simultaneous or sequential co-administration is desirable as opiate and SP systems overlap with both serotonin and NE systems. Any increased sensitivity of the opiate and/or SP systems also has an effect on serotonin and NE systems. The enhanced sensitivity of the serotonin or NE systems that is a result of counteradaptive therapy generates an enhanced response to either SSRI or NRI therapy.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents. All references cited herein are hereby incorporated by reference in their entirety.

### REFERENCES

U.S. Patent References:

U.S. Pat. No. 3,980,641 September, 1976 Monkovic Process for the preparation of 14-hydroxymorph . . .
U.S. Pat. No. 4,059,693 November, 1977 Stewart Analgesic action of substance P
U.S. Pat. No. 4,217,353 August, 1980 Smith Method for inducing anorexia
U.S. Pat. No. 4,361,553 November, 1982 Loh Therapeutic uses of dynorphin
U.S. Pat. No. 4,377,595 March, 1983 Wurtman Process for reducing depression
U.S. Pat. No. 4,421,744 December, 1983 Gormley Aminoacyl derivatives
U.S. Pat. No. 4,462,941 July, 1984 Lee Dynorphin amide analogs
U.S. Pat. No. 4,464,358 August, 1984 Cox Homocyclic derivatives
U.S. Pat. No. 4,468,383 August, 1984 Rodbard Dimeric enkephalins
U.S. Pat. No. 4,472,305 September, 1984 Hansen Hexapeptide amides
U.S. Pat. No. 4,474,767 October, 1984 Cotton Peptide and pseudopeptide derivatives
U.S. Pat. No. 4,474,799 October, 1984 Greenberg Enkephalinase inhibitors
U.S. Pat. No. 4,476,117 October, 1984 Morley Dimers of peptide amides
U.S. Pat. No. 4,477,457 October, 1984 Smith Method for inducing anorexia using nalmetrene
U.S. Pat. No. 4,478,840 October, 1984 Smith Appetite suppressing compositions and methods
U.S. Pat. No. 4,481,191 November, 1984 Wei Method for controlling blood pressure
U.S. Pat. No. 4,504,492 March, 1985 Wilkinson Pharmaceutical amides, and preparation, . . .
U.S. Pat. No. 4,518,711 May, 1985 Hruby Conformationally constrained cyclic enkephalin . . .
U.S. Pat. No. 4,537,878 August, 1985 Plotnikoff Process for using endogenous enkephalins and . . .
U.S. Pat. No. 4,546,103 October, 1985 Huebner Method of treating weight loss disorders
U.S. Pat. No. 4,567,185 January, 1986 Sackner Endorphin blockage
U.S. Pat. No. 4,573,995 March, 1986 Chen Transdermal therapeutic systems . . .
U.S. Pat. No. 4,600,718 July, 1986 Huebner Method of treating weight loss disorders
U.S. Pat. No. 4,638,046 January, 1987 Verdini Retro-inverso C-terminal hexapeptide analogues . . .
U.S. Pat. No. 4,680,283 July, 1987 Veber Analogs of substance P and eledoisin
U.S. Pat. No. 4,684,620 August, 1987 Hruby Cyclic polypeptides having muspecificity
U.S. Pat. No. 4,684,624 August, 1987 Hosobuchi Method of treating cerebral ischemia
U.S. Pat. No. 4,689,332 August, 1876 McLaughlin Growth regulation and related applications . . .
U.S. Pat. No. 4,757,049 July, 1988 Plotnikoff Process for using endogenous enkephalins and . . .
U.S. Pat. No. 4,758,562 July, 1988 Adler Method for inducing hypothermia and/or . . .
U.S. Pat. No. 4,761,429 August, 1988 Blum Enkephalinase and endorphinase inhibitors as . . .
U.S. Pat. No. 4,767,764 August, 1988 Ciganek Alkoxyalkyl and alkylmercaptoalkyl substituted . . .
U.S. Pat. No. 4,801,614 January, 1989 Plotnikoff Process for using endogenous enkephalins and . . .
U.S. Pat. No. 4,816,586 March, 1989 Portoghese Delta opioid receptor antagonists
U.S. Pat. No. 4,839,465 June, 1989 Singh Di-(D-tryptophyl and/or tetrahydropyridoindol . . .
U.S. Pat. No. 4,857,533 August, 1989 Sherman Method of treatment for autoimmune diseases
U.S. Pat. No. 4,888,346 December, 1989 Bihari Method for the treatment of persons . . .
U.S. Pat. No. 4,910,152 March, 1990 Meyers Method for binding opioid receptors
U.S. Pat. No. 5,013,739 May, 1991 Bihari Method of treating chronic fatigue syndrome . . .
U.S. Pat. No. 5,013,740 May, 1991 Glover Method for treating emotional numbness . . .
U.S. Pat. No. 5,017,689 May, 1991 Hruby Dynorphin analogs specific for kappa opioid . . .
U.S. Pat. No. 5,025,018 June, 1991 Faden Central nervous system injury treatment . . .
U.S. Pat. No. 5,028,612 July, 1991 Glover Method for treating emotional numbness
U.S. Pat. No. 5,051,428 September, 1991 Horwell 2-amino-4 or 5-methoxycyclohexyl amides . . .
U.S. Pat. No. 5,096,715 March, 1992 Sinclair Method and means for treating alcoholism by . . .
U.S. Pat. No. 5,137,873 August, 1992 Yankner Substance P and tachykinin agonists . . .
U.S. Pat. No. 5,141,962 August, 1992 Cosquer Amine compounds
U.S. Pat. No. 5,166,136 November, 1992 Ward Spirolactam containing peptides
U.S. Pat. No. 5,175,144 December, 1992 Walser Method of retarding the progression of chronic . . .
U.S. Pat. No. 5,266,574 November, 1993 Zagon Growth regulation and related applications . . .
U.S. Pat. No. 5,326,751 July, 1994 Haaseth Enkephalin analogs
U.S. Pat. No. 5,332,818 July, 1994 Nagase Immunosuppressive agent and process . . .
U.S. Pat. No. 5,352,680 October, 1994 Portoghese Delta opioid receptor antagonist to block opioid
U.S. Pat. No. 5,356,900 October, 1994 Bihari Method of treating chronic herpes virus . . .
U.S. Pat. No. 5,376,662 December, 1994 Ockert Method of attenuating nerve injury induced pain
U.S. Pat. No. 5,395,398 March, 1995 Rogozinski Microelectric apparatus for the antisepsis, . . .
U.S. Pat. No. 5,410,019 April, 1995 Coy Therapeutic peptides
U.S. Pat. No. 5,411,965 May, 1995 Reid Use of delta opioid receptor antagonists to treat . . .
U.S. Pat. No. 5,426,112 June, 1995 Zagon Growth regulation and related applications . . .
U.S. Pat. No. 5,464,841 November, 1995 Portoghese Use of delta opioid receptor antagonists to treat . . .
U.S. Pat. No. 5,512,593 April, 1996 Dante Composition and method of treating depression . . .
U.S. Pat. No. 5,552,404 September, 1996 Chang Opioid compounds and methods for using same
U.S. Pat. No. 5,574,159 November, 1996 Chang Opioid compounds and methods for making . . .
U.S. Pat. No. 5,602,099 February, 1997 Schiller delta. opioid receptor antagonists
U.S. Pat. No. 5,610,271 March, 1997 Dooley Kappa receptor selective opioid peptides
U.S. Pat. No. 5,616,562 April, 1997 Murphy Methods and compositions using substance P to . . .
U.S. Pat. No. 5,631,263 May, 1997 Portoghese Use of delta opioid receptor antagonists to treat . . .
U.S. Pat. No. 5,658,908 August, 1997 Chang Opioid diarylmethylpiperazines and piperdines
U.S. Pat. No. 5,681,830 October, 1997 Chang Opioid compounds
U.S. Pat. No. 5,807,827 September, 1998 Lee Des-Tyr dynorphin analogues
U.S. Pat. No. 5,817,628 October, 1998 Kreek Dynorphin a suppression of natural killer activity
U.S. Pat. No. 5,821,219 October, 1998 Grandy Opioid antagonists and methods of their use
U.S. Pat. No. 5,852,032 December, 1998 Mason Method of treating nicotine dependence
U.S. Pat. No. 5,872,097 February, 1999 Fholenhag Oligopeptides with affinity to opioid receptors
U.S. Pat. No. 5,891,842 April, 1999 Kream Methodology for eliciting an analgesic response
U.S. Pat. No. 5,922,361 July, 1999 Bieser Folic acid enriched dietary supplement . . .
U.S. Pat. No. 5,922,887 July, 1999 Dondio Diaryldiamine derivatives and their use as delta
U.S. Pat. No. 5,945,508 August, 1999 Witten Substance P treatment for immunostimulation
U.S. Pat. No. 5,958,429 September, 1999 Wong Potentiation of serotonin response
U.S. Pat. No. 5,965,701 October, 1999 Junien Kappa receptor opioid peptides
U.S. Pat. No. 5,972,938 October, 1999 Rupniak Method of preventing psychoimmunological . . .
U.S. Pat. No. 5,990,125 November, 1999 Howard NK-1 receptor antagonists for the treatment . . .
U.S. Pat. No. 5,998,376 December, 1999 Witten Substance P treatment for immunostimulation
U.S. Pat. No. 6,063,758 May, 2000 Lappi Substance P-Saporin (SP-SAP) conjugates and . . .
U.S. Pat. No. 6,096,513 August, 2000 Bell Polynucleotides encoding KAPPA opioid . . .
U.S. Pat. No. 6,136,780 October, 2000 Zagon Control of cancer growth through the interaction . . .
U.S. Pat. No. 6,146,835 November, 2000 Kieffer Human kappa opioid receptor, nucleic acids . . .
U.S. Pat. No. 6,159,970 December, 2000 Berg Combination of a monoamine oxidase inhibitor . . .
U.S. Pat. No. 6,159,971 December, 2000 Berg Combination of a 5-HT reuptake inhibitor . . .
U.S. Pat. No. 6,159,972 December, 2000 Berg Combination of a selective 5-HT₁A antagonist . . .
U.S. Pat. No. 6,166,020 December, 2000 Howard Aryl and heteroaryl alkoxynaphthalene . . .
U.S. Pat. No. 6,174,891 January, 2001 Nagase Antipruritic agent
U.S. Pat. No. 6,177,450 January, 2001 Garret Synergizing combination having an antagonist . . .
U.S. Pat. No. 6,191,126 February, 2001 Gamache Topical use of .kappa. opioid agonists to treat . . .
U.S. Pat. No. 6,221,878 April, 2001 Tyers Method for treatment of depression
U.S. Pat. No. 6,262,104 July, 2001 Dondio Diarylalkenylamine derivatives
U.S. Pat. No. 6,271,239 August, 2001 Porotghese Delta opioid receptor-selective benzylidene- . . .
U.S. Pat. No. 6,277,852 August, 2001 Howard piperazinyl 5-HT1 agonists and antagonists
U.S. Pat. No. 6,284,769 September, 2001 Dunn Nonpeptide kappa receptor antagonists
U.S. Pat. No. 6,288,074 September, 2001 Bihari Method of treating lymphoproliferative syndrome
U.S. Pat. No. 6,294,520 September, 2001 Naito Material for passage through the blood-brain . . .
U.S. Pat. No. 6,303,578 October, 2001 Zadina Mu-opiate receptor peptides
U.S. Pat. No. 6,306,876 October, 2001 Carson 4-[aryl(8-azabicyclo[3.2.1]octan-3-yl)]amino . . .
U.S. Pat. No. 6,316,461 November, 2001 Nagase Antipruritic
U.S. Pat. No. 6,337,319 January, 2002 Wang mu.-Selective opiate peptides
U.S. Pat. No. 6,369,074 April, 2002 Howard Aminomethylene substituted non-aromatic . . .
U.S. Pat. No. 6,384,044 May, 2002 Bihari Method of treating cancer of the prostate
U.S. Pat. No. 6,410,773 June, 2002 Yasohara Sulfonic acid ester derivatives, method for . . .
U.S. Pat. No. 6,436,959 August, 2002 Carson 4-[aryl(piperidin-4-yl)]aminobenzamides
U.S. Pat. No. 6,437,093 August, 2002 Lieberman Methods of treatment comprising administration
U.S. Pat. No. 6,440,987 August, 2002 Nagase Antipruritic
U.S. Pat. No. 6,451,803 September, 2002 Howard N-acyl and n-aroyl aralkyl amides useful in . . .
U.S. Pat. No. 6,451,806 September, 2002 Farrar Methods and compositions involving opioids . . .
U.S. Pat. No. 6,455,536 September, 2002 Brotchie Treatment of dyskinesia
U.S. Pat. No. 6,462,048 October, 2002 Howard Benzyl(idene)-lactam derivatives . . .
U.S. Pat. No. 6,500,818 December, 2002 Bernstein Naphthalenecarboxamides as tachykinin receptor . . .
U.S. Pat. No. 6,500,918 December, 2002 Ezrin Conjugate comprising an antinociceptive agent . . .
U.S. Pat. No. 6,509,014 January, 2003 De Lacharriere Therapeutic/cosmetic compositions comprising . . .
U.S. Pat. No. 6,514,975 February, 2003 Maw Anti-inflammatory piperazinyl-benzyl-tetrazole . . .
U.S. Pat. No. 6,518,295 February, 2003 Merce-Vidal Utilization of aryl(or heteroaryl)azolylcarbinol . . .
U.S. Pat. No. 6,525,062 February, 2003 Levine Method of treating pain using nalbuphine and . . .
U.S. Pat. No. 6,528,518 March, 2003 Carlezon Treatment of depression with kappa receptor . . .
U.S. Pat. No. 6,534,514 March, 2003 Portoghese Kappa opioid receptor antagonists
U.S. Pat. No. 6,541,478 April, 2003 O'Malley Smoking cessation treatments using naltrexone . . .
U.S. Pat. No. 6,552,036 April, 2003 Boyd 3-(Diarylmethylene)-8-azabicyclo[3.2.1]octane . . .
U.S. Pat. No. 6,559,159 May, 2003 Carroll Kappa opioid receptor ligands
U.S. Pat. No. 6,576,638 June, 2003 Pompei Use of substance P antagonists in the treatment . . .
U.S. Pat. No. 6,576,762 June, 2003 Hoffman Heteroaromatic substituted amides with . . .
U.S. Pat. No. 6,583,151 June, 2003 Nagase Remedies for drug addiction
U.S. Pat. No. 6,586,432 July, 2003 Bernstein Neurokinin-1 receptor antagonists
U.S. Pat. No. 6,586,443 July, 2003 Bihari Method of treating multiple sclerosis
U.S. Pat. No. 6,596,692 July, 2003 Burman Substance P analogs for the treatment of cancer
U.S. Pat. No. 6,602,874 August, 2003 Howard Heterocyclic carboxamides
U.S. Pat. No. 6,602,981 August, 2003 Ezrin Antinociceptive agent derivative
U.S. Pat. No. 6,610,825 August, 2003 Ezrin Method for alleviating pain or providing . . .
U.S. Pat. No. 6,624,313 September, 2003 DeVries Process for preparing hydroxypyrrolidinyl . . .
U.S. Pat. No. 6,627,627 September, 2003 Howard Aralkyl and aralkylidene heterocyclic lactams . . .
U.S. Pat. No. 6,642,233 November, 2003 Ducoux 1-Phenacyl-3-phenyl-3-(piperidylethyl) . . .
U.S. Pat. No. 6,642,240 November, 2003 Alvaro Treating emesis in a mammal
U.S. Pat. No. 6,696,457 February, 2004 Clarke Morphinoid compounds
U.S. Pat. No. 6,713,488 March, 2004 Sadee Neutral antagonists and use thereof in treating . . .
U.S. Pat. No. 6,716,449 April, 2004 Oshlack Controlled release compositions containing . . .
U.S. Pat. No. 6,737,397 May, 2004 Zagon Control of cancer growth . . .
U.S. Pat. No. 6,740,659 May, 2004 Brotchie Treatment of dyskinesia
U.S. Pat. No. 6,770,654 August, 2004 Sakami Indole derivatives and use thereof in medicines
U.S. Pat. No. 6,790,854 September, 2004 Tsushima Diphenylalkylamine derivatives useful as opioid . . .
U.S. Pat. No. 6,825,205 November, 2004 Kyle N-substituted hydromorphones and the use . . .
U.S. Pat. No. 6,838,580 January, 2005 Okada Opioid derivative
U.S. Pat. No. 6,841,551 January, 2005 Nimmo Brain, spinal, and nerve injury treatment
   Published International Applications:
WO 95/16679
WO 95/18124
WP 95/23798
http://neurotransmitter.net/newdrugs.html “Treatments for Depression and Anxiety”
http://anaspec.com/products/productcategory.asp?id=192 “Substance P and analogues, from AnaSpec (San Jose, Calif.).
http://www.acnp.org/g4/GN 401000110/CH108.html, Post, R M, et al., “the Neurobiology of Treatment-Resistant Disorders—Cocaine Induced Behavioral Sensitization and Its Cross Sensitization To Stress”.
http://osiris.sunderland.ac.uk/autism/naltrexo.htm Naltrexone—A Short Overview.
http://www.komabiotech.co.kr/product/neuro/categorv/mu_Receptor.htm Category>Opioid Receptor Ligands>μReceptor
http://www.komabiotech.co.kr/product/neuro/categorv/delta_Receptor.htm Category>Opioid Receptor Ligands>Delta Receptor
Adessi, C., et al., “Converting a peptide into a drug: Strategies to improve stability and bioavailability”, Curr Med Chem, 2002 May; 9(9): 963-978.
Alcaraz, C, et al., “Chronic naloxone treatment induces supersensitivity to a mu but not to a kappa agonist at the hypothalamus-pituitary-adrenocortical axis level” J Pharmacol Exp Ther. 1993 Sep. 1; 266(3): 1602-1606.
Andersson, K E, “Advances in the pharmacological control of the bladder” Exper Physiol. 1999; 84(1): 195-213.
Arango, V, et al., “Serotonin 1A receptors, serotonin transporter binding and serotonin transporter mRNA expression in the brainstem of depressed suicide victims” Neuropsychopharmacology. 2001 December; 25(6): 892-903.
Argyropoulos, S V, et al., “Substance P antagonists: novel agents in the treatment of depression” Exper Opin Investig Drugs. 2000 August; 0(8): 1871-1875.
Arnsten, A F, et al., “Postsynaptic alpha-2 receptor stimulation improves memory in aged monkeys: indirect effects of yohimbe versus direct effects of clonidine” Neurobiol Aging. 1993 November-December; 14(6): 597-603.
Artigas, F, et al., “Acceleration of the effect of selected antidepressant drugs in major depression by 5-HT1A antagonists.” Trends Neurosci. 1996 September; 19(9): 378-83.
Artigas, F, “Role of 5-HT_{1A} and 5-HT_{2A} Serotonergic Receptors in the Treatment of Depression and Schizophrenia” Methods and Findings in Experimental and Clinical “Pharmacology, 2004; 25(Suppl A).
Avidor-Reiss, T, et al., “Adenylylcyclase Supersensitization in μ-Opioid Receptor-transfected Chinese hamster Ovary Cells Following Chronic Opioid Treatment” J Biol. Chem. 1995 Dec. 15; 270(50): 29732-29738.
Baby, S, et al., “Substance P antagonists: the next breakthrough in treating depression?” REVIEW Article. J Clin Pharmacy & Therapeutics. 1999 December; 24(6): 461-9.
Badgery-Parker, T, et al., “A novel, selective radioligand, [1251]-Lys5,Tyr(I2)7,MeLeu9,Nle10]-NKA(4-10), for the tachykinin NK-2 receptor” Regul Pept. 1993 Jul. 2; 46(1-2): 455-7.
Bailey, A, et al., “Quantitative autoradiography of adenosine receptors in brains of chronic naltrexone-treated mice” British J. Pharmacol. 139: 1187-1195.
Bang, R et al., “Neurokinin-1 receptor antagonists CP-96,345 and L-733,060 protect mice from cytokine-mediated liver injury” J Pharmacol Exp Ther, 2003 April; 305(1): 31-9.
Belcheva, M M, et al., “Antagonist-induced transient down-regulation of delta-opioid receptors in NG108-15 cells” Molecular Pharmacol. 1992; 42(3): 445-452.
Bennett, V J, et al., “Analysis of fluorescently labeled substance P analogs: binding, imaging and receptor activation” BMC Chemical Biology. 2001; 1:1.
Bennett, V J, et al., “A Novel Mechanism of Neurokinin-1 Receptor Resensitization” J Pharmcol Exp Ther. 2002; 303(3): 1155-1162.
Bernard, J M, et al., “Comparison of intravenous and epidural clonidine for postoperative patient-controlled analgesia” Anesthesia & Analgesia; 81: 706-712.
Bhatia, M, et al., “Tachykinins as Therapeutic Targets in Inflammation” Curr Med Chem—Anti-Imflammatory & Anti-Allergy Agetns. 2003 March; 2(1): 12-27.
Bhatia, P, et al., “Dexmedetomidine: A New Agent In Anaesthesia & Critical Care Practice” from: The Indian Anaesthetists'Forum, 2002 April—web address: http://www.theiaforum.org/apr2002.htm.
Bidlack, J M, “Detection and Function of Opioid Receptors on Cells from the Immune system” Clinical & Diagnostic Lab Immunology. 2000 September; 7(5): 719-723.
Bihari, B., et al., “Low Dose Naltrexone in the Treatment of Acquired Immune Deficiency Syndrome”, June, 1988, Oral presentation at the IV International AIDS Conference, Stockholm, Sweden.
Bilkei-Gorzo, et al., “Diminished Anxiety- and Depression-Related Behaviors in Mice with Selective Deletion of the Tac1 Gene” J. Neurosci. 2002 Nov. 15; 22(22): 10046-52.
Bilsky, E J., et al., “SNC 80, a selective, nonpeptidic and systemically active opioid delta agonist” Pharmacology & Experimental Therapeutics, 1995 Apr. 1; 273(1): 359-366.
Binder, W, PhD, et al., “Analgesic and Antiinflammatory Effects of Two Novel [kappa]-Opioid Peptides” Anesthesiology. 2001; 94(6): 1034-1044.
Biver, F, et al., “Serotonin 5-HT2 receptor imaging in major depression: focal changes in orbito-insular cortex” Br J Psychiatry. 1997 November; 171: 444-8.
Blier, P, et al., “Selective activation of postsynaptic 5-HT1A receptors induces rapid antidepressant response.” Neurosychopharmacol. 1977 May, 16(5): 333-8.
Blier, P, et al., “Is there a role for 5-HT1A agonists in the treatment of depression?” Biol Psychiatry. 2003 Feb. 1; 53(3): 193-2036.
Blier, P, et al., “The pharmacology of putative early-onset antipdressant strategies” Eur Neuropsychopharmacol. 2003 March; 13(2): 57-66.
Blier, P, et al., “Electrophysiological investigation of the adaptive response of the 5-HT system fo the administration of 5-HT1A receptor agonists” J Cardiovasc Pharmacol. 1990; 15 Suppl 7: S42-8.
Blier, P, “Pharmacology of rapid-onset antidepressant treatment strategies” J Clin Psychiatry. 2001; 62 Suppl 15: 12-7.
Bodkin, J A, “Buprenorphine treatment of refractory depression” J Clin Psychopharmacol. 1995 February; 15(1): 49-57.
Boehm S, “Presynaptic alpha2-adrenoceptors control excitatory, but not inhibitory, transmission at rat hippocampal synapses” J Physiol (Lond). 1999 Sep. 1; 519(Pt 2): 439-449.
Bondy, B, et al., “Substance P serum levels are increased in major depression: preliminary results” Psychiatry. 2003 Mar. 15; 53(6): 538-42.
Borsini, F., et al., “BINT 17, a 5-HT_{1A} receptor agonist/5-HT_{2A} receptor antagonist, directly activates postsynaptic 5HT inhibitory responses in the rat cerebral cortex” Naunyn-Schmiedeberg's Archives of Pharmacology (Historical Archive), 1995 September; 352(3): 283-290.
Boyce, S., et al., “Intra-amygdala injection of the substance P[NK(1) receptor] antagonist L-760735 inhibits neonatal vocalizations in guinea-pigs” Neuropharmacology, 2001 July; 41(1): 130-7.
Briner, K, et al., “New approaches to rapid-onset antidepressants” Curr Pharm Des. 1998 August; 4(4): 291-302.
Broom, D C, et al., “BU48: A Novel Buprenorphine Analog That Exhibits delta-Opioid-Mediated Convulsions but No delta-Opioid-Mediated Antinociception in mice” J Pharmacol Exp her. 2000; 294: 1195-1200.
Broom, D C, et al., “Behavioral effects of delta-opioid receptor agonists: potential andepressants?” Jpn J Pharmacol, 2002 September; 90(1): 106.
Burnett, F E, “The effect of naloxone on adrenocorticotropin and cortisol release: evidence for a reduced response in depression” J Affect Disord. 1999 June; 53(3): 263-8.
Butelman, E R, et al., “The plant-derived hallucinogen, salvinorin A, produces kappa-opioid agonist-like discriminative effects in rhesus monkeys” Psychopharmacol. 2003, Oct. 30.
Cadieux, R J, “Practical Management of Treratmment-Resistant Depression” American Family Physician. 1998 December: pg 3-4.
Cao, T, et al., “Neurokinin-1 Receptor Agonists Are Involved in Mediating Neutrophil Acculumation in the Inflamed, But Not Normal, Cutaneous Microvasculature: An In Vivo Study using Neurokinin-Receptor Knockout Mice” J Immunology. 2000; 164: 5424-5429.
Chandrashekar, I R, et al., “Tachykinin Peptide Neurokinin A Bound to Lipid Micelles” Biophysical Journal. 2004; 85: 4002-4011.
Charney, D S, et al., “Alpha-2 adrenergic receptor sensitivity and the mechanism of action of antidepressant therapy. The effect of long-term amitriptyline treatment” Br J Psychiatry. 1983; 142: 265-275.
Chavkin, C, et al., “Salvinorin A, an Active Component of the Hallucinogenic Sage Salvia divinorum is a Highly Efficacious K-Opioid Receptor Agonist: Structural and Functional Considerations” J Pharmacol Exp Ther. 2004; 308(3): 1197-1203.
Chorev, M, et al., “Toward nonpeptidal substance P mimetic analogues: design, synthesis and biological activity” Biopolymers. 1991; 31(6): 725-33.
Cleare, A J, et al., “Do noradrenergic reuptake inhibitors affedt serotonergic function in depression?” Psychopharmacol (Berl). 1997 December; 134(4): 406-10.
Cohen, M R, et al., “High dose naloxone in depression” Biol Psychiatry. 1984 June; 19(6): 825-32.
Cote, T E, et al., “Naltrexone-induced upregulation of mu opioid receptors on 7315c cell and brain membraines: enhancement of opioid efficacy in inhibiting adenylyl cyclase” J Pharmacol Exp Ther. 1993 Oct. 1; 267(1): 238-244.
Coull, J T, “Pharmacological manipulations of the alpha 2-noradrenergic system. Effects on cognition” Drugs Aging. 1994 August; 5(2): 116-26
Courteix, C, et al., “RP-67580, a specific tachykinin NK1 receptor antagonist, relieves chronic hyperalgesia in diabetic rats” Eur J. Pharmacol. 1993 Sep. 14; 241(2-3): 267-70.
Crabtree, B L, “Review of naltrexone, a long-acting opiate antagonist” Clin Pharm. 1984 May-June; 3(3): 273-80.
Crowley, T J, et al., “Naltrexone-induced dysphoria in former opioid addicts” Am J Psychiatry. 1985 September; 142(9): 1081-4.
Dalman, F C, et al., “Kappa-Opioid tolerance and dependence in cultures of dopaminergic midbrain neurons” J. Neurosci. 1999 Jul. 15; 19(14): 5750-7.
De Groote L, et al., “Role of extracellular serotonin levels in the effect of 5-HT(1B) receptor blockade” Psychopharmacol (Berl). 2003 May; 167(2): 153-8.
Del Campo, M, “The use of the plasma concentration-effect relationship as a tool for the study of the mechanism of action of naloxone effects on mood and endocrine function” J Psychopharmacol (Oxf). 1997; 11(4): 361-5.
Del Campo, M, et al., “Diurnal variations in endocrine and psychological responses to 0.2 mg/kg naloxone administration in patients with major depressive disorder and matched controls” J Affect Disord. 2000 January-March; 57(1-3): 37-47.
Delgado, P L, et al., “Role of norepinephrine in depression” J Clin Psychiatry. 2000; 61 Suppl 1: 5-12.
DeVane, C Lindsey, “Substance P: A New Era, a New Role” Pharmacotherapy. 2001; 21(9): 1061-1069.
De Vry, J, “5HT-1A receptor agonists: recent developments and controversial issues” Psychopharmacol (Berl). 1995 September; 121(1): 1-26.
Diener, H-C, “RPR 100893, a substance-P antagonist, is not effective in the treatment of migraine attacks” Cephalgia. 2004 April; 23(3): 183-185.
Drapeau, G, et al., “Selective agonists for substance P and neurokinin receptors” Neuropeptides. 1987 July; 10(1); 43-54.
Dzivenu, O K, et al., “Cancer and the Immune System: The Vital Connection” Cancer Research Institute. http://cancerresearch.org/immunology/immuneindex.html. Copyright ®Cancer Research Institute 2003.
Eikelboom, R “Naloxone, naltrexone and body temperature” Life Sci. 1987 Mar. 16; 40(11): 1027-32.
Ellen, M, et al., “Chronic opioid antagonist administration upregulates mu opioid receptor binding without altering mu opioid receptor mRNA levels” Molecular Brain Res. 1995 November; 33(2): 351-355.
Extein, I, et al., “Methadone and Morphine in Depression” (Fair Oaks Hospital, Summit, N.J.); from http://opioids.com/antidepressant/.
Fagergren, Pernilla, “Cocaine effects on striatal dynorphin and CART neuropeptides: Association of mood disorder” from Doktorsavhandling vid Karolinska Institutet—Fredagen den 6 Juni 2003, kl. 9.00. Leksells auditorium, Medicinhistoriska museet, Karolinska Sjukhuset. ISBN: 91-7349-515-8.
Fava, G A, “Can long-term treatment with antidepressant drugs worsen the course of depression?” J Clin Psychiatry 2003 February; 64(2): 123-33.
Fawcett, J, et al., “Review of the results from clinical studies on the efficacy, safety and tolerability of mirtazapine for the treatment of patients with major depression” J Affect Disord. 1998 December; 51(3): 267-85.
File, SE. “NKP608, an NK1 receptor antagonist, has an anxiolytic action in the social interaction test in rats” Psychopharmacology (Berl) 2000 September; 152(1): 105-9.
Flaumenhaft, R, “Platelets feel your pain” Blood. 2004; 104(4): 913.
Fletcher, A, et al., “Electrophysiological, biochemical, neurohormonal and behavioural studies with WAY-100635, a potent, selective and silent 5-HT1A receptor antagonist” Behav Brain Res. 1996; 73(1-2): 337-53.
Fornal, C A, et al., “Effects of the putative 5-hydroxytryptamine1A antagonists BMY 7378, NAN 190 and (−)-propanolol on serotonergic dorsal raphe unit activity in behaving cats” J Pharmacol Exp Ther. 1994 Sep. 1; 270(3): 1359-1366.
Fornal, C A, et al., “WAY-100635, a potent and selective 5-hydroxytryptamine1A antagonist, increases serotonergic neuronal activity I behaving cats: comparison with (S)-WAY-100135” 1996 Aug. 1; 278(2): 752-762.
Friess, H., et al., “Neurokinin-1 receptor expression and its potential effects on tumor growth in human pancreatic cancer” Lab Invest, 2003 May; 83(5): 731-42.
Gardier, A M, et al., “5-HT1B serotonin receptors and antidepressant effects of selective serotonin reuptake” C R Acad Sci III. 2001 May; 324(5): 433-41.
Gaveriaux-Ruff, C, et al., “Immunosuppression byδ-Opioid Antagonist Naltrindole: δ- and Tripleμ/δ/κ-Opioid Receptor Knockout Mice Reveal a Nonopioid Activity” J Pharmacol Exp Ther. 2001 September; 298(3): 1193-1198.
Gobert, A, et al., “Alpha2-adrenergic receptor blockade markedly potentiates duloxetine- and fluoxetine-induced increases in noradrenaline, dopamine, and serotonin levels in the frontal cortex of freely moving rats” J. Neurochem. 1997 December; 69(6): 2616-9.
Gobert, A, et al., “Buspirone modulates basal and fluoxetine-sitmulated dialysate levels of dopamine, noradrenaline and serotonin in the frontal cortex of freely moving rats: activation of serotonin1A receptors and blockade of alpha2-adrenergic receptors underlie its actions” Neurosci. 1999; 93(4): 1251-62.
Gorman, J M, et al., “Noradrenergic approaches to antidepressant therapy” J Clin Psychiatry. 2000; 61 Suppl 1: 13-16.
Greenwald, M., et al., “Human Pharmacology of the Opioid Neuropeptide Dynorphin A(1-13)” Pharmacol Exper Ther. 1997; 281(3): 1154-1163.
Haddjeri, N, et al., “Effect of the alpha-2 adrenoceptor antagonist mirtazapine on the 5-hydroxytryptamine system in the rat brian.” Pharmacol Exp Ther. 1996 May 1; 277(2): 861-871.
Haddjeri, H, et al., “Long-Term Antidepressant Treatments Result in a Tonic Activation of Forebrain 5-HT_{1A} Receptors” J Neuroscience. 1998 Dec. 1; 18(23): 10150-10156.
Hay, D, “Tachykinin Antagonists” from New Drugs for Asthma, Allergy and COPD.
Hansel T T, Barnes P J (eds): Prog Respir Res. Basel, Karger, 2001, vol. 31: 145-150.
Hayes, R H, et al., “The selectivity of the opioid antagonist, naltrindole, for delta-opioid receptors” J Pharm Pharmacol. 1990 March; 42(5): 358-9.
Heidi, M B, et al., “Receptor-selective changes in μ, δ- and κ-opioid receptors after chronic naltrexone treatment in mice” Eur J. Neurosci. 2003 March; 17(4): 1006.
Helmstetter, F J, et al., “Strain Differences in Reversal of Conditional Analgesia by Opioid Antagonists” Behavioral Neurosci. 1987; 101(5): 735-737.
Hensler, J G, et al., “Effect of chronic Serotonin-2 Receptor Agonist or Antagonist Administration on Serotonin 1A Receptor Sensitivity.” Neuropsychopharmacology. 1998; 19: 354-364.
Hirschfeld, R M, “History and evolution of the monoamine hypothesis of depression” J Clin Psychiatry. 2000; 61 Suppl 6: 4-6.
Hjorth, S, et al., “Serotonin autoreceptor function and antidepressant drug action” J Psychopharmacol. 2000 June; 14(2): 177-85.
Hollt, V, “Multiple endogenous opioid peptides” Trends in Neuroscience. 1983; 16:24-26
Holter, S M, et al., “Effects of opiate antagonist treatmente on the alcohol deprivation effect in Ion-term ethanol-experienced rats” Psychopharmacol. 1999 August; 145(4): 360-9.
Hurd, Y. “The Dynorphin/Kapp Mesolimbic System in Heroin Abuse” R01 DA 15446-01 NIDA from Karolinska Institute. From web page: http://www.varimed.hu/bridges/abs_{—}8_{—}4.htm.
Hurd, Y L, “Subjects with major depression or bipolar disorder show reduction of prodynorphin mRNA expression in discrete nuclei of the amygdaloid complex” Mol Psychiatry. 2002; 7(1): 75-81.
Jang, T S, et al., “Structure-Contractile Activity Relationships of Neurokinin A on Guinea Pig Tracheal Smooth Muscle” Bull Korean Chem Soc. 1999; 20(2); 1-4.
Jenab, S, et al., “Ethanol and naloxone differentially upregulate delta opioid receptor gene expression in neuroblastoma hybrid (NG108-15) cells” Molecular Brain Res. 1994; 27(1): 95-102.
Joca, S R, et al., “Activation of post-synaptic 5-HT(1A) receptors in the dorsal hippocampus prevents learned helplessness development” Brain Res. 2003 Jul. 18; 978(1-2): 177-84.
Jouvet, M, et al., “Noradrenaline and cerebral aging’ Encephale. 1991 May-June; 17(3): 187-95.
Kamali, F. “Osanetant Sanofi-Synthelabo.” Curr Opin Investig Drugs. 2001 July; 2(7): 950-6.
Kennett, G A, (Assistant Director, Neurobehavioural Research, SmithKline Beecham Pharmaceuticals, New Frontiers Science Park, Third Avenue, Harlow, Essex, CM19 5AW, UK.) “Serotonin Receptors and their Function”
Kidd, B L, et al., “Inhibition of inflammation and hyperalgesia in NK-1 receptor knock-out mice” Neuroreport. 2003 Dec. 2; 14(17): 2189-92.
Koek, W, et al., “F 11440, a potent, selective, high efficacy 5-HT1A receptor agonist with marked anxiolytic and antidepressant potential” J Pharmacol Exp Ther. 1998 October; 237(1): 266-83.
Koek, W, et al., “5-HT(1A) receptor activation and antidepressant-like effects. F 13714 has high efficacy and marked antidepressant potential” Eur J. Pharmacol. 2001 May 25; 420(2-3): 103-112.
Kondo, I, et al., “Spinal Morphine Withdrawal-Induced Hyperalgesia Is Mediated by Increased Release of Substance P and Activaton of NK1 Receptors” Anesthesiology. 2003; 99: A1095.
Kramer, T, “Substance P Research in Psychiatry” XXIInd Congress of the Collegium Innternationale Neuro-Psychopharmacologicum—Depression and Anxiety Disorders.
Kreek, M J, et al., “Dynorphin A (1-13) Analgesia in Opioid-Treated Patients with Chronic Pain: A Controlled Pilot Study” Clin Drug Investig. 1999 January; 17(1): 33-42.
Kshirsagar, T A, et al., “14-Desoxy Analogues of Naltrindole and 7-Spiroindanyloxymorphone: The Role of the 14-Hydroxy Group at 6 Opioid Receptors” J Med. Chem. 1998; 41: 2657-2660.
Kudlacz E M, et al., “Combined tachykinin receptor antagonists for the treatment of respiratory diseases” Expert Opinion on Invest Drugs, 1998 July; 7(7): 1055-1062.
Lai, J, et al., “Neuropathic Pain: The Paradox of Dynorphin: Review” Molecular Interventions. 2001; 1: 160-167.
Laughlin, T M, et al., “Mechanisms of Induction of Persistent Nociception by Dynorphin” J Pharmacol Exp Ther. 2001 October; 299(1): 6-11.
Lecci, A, “Pharmacology and Function of Tachykinin Receptors” Tocris Reviews. 2003 September; 24: 1-12.
Lepaul, E., et al., “Early desensitization of somato-dendritic 5-HT1A autoreceptors in rats treated with fluoxetine or paroxetine” Naunyn Schmiedebergs Arch Pharmacol. 1995 August; 352(2): 141-8.
Li, X, et al., “Opioid-Induced Hyperalgesia and Incisional Pain” Anesth Analg. 2001; 93: 204-209.
Lieb, K, et al., “Effects of the Neuropeptide Substance P on Sleep, Mood and Neuroendocrine Measurers in Healthy Young Men” Neuropsychopharmacology. 2002; 27: 1041-1049.
Ling, G S, et al., “Naloxonazine actions in vivo” 1986 Sep. 23; 129(1-2): 33-8.
Linner, L, et al., “Locus coeruleus neuronal activity and noradrenaline availability in the frontal cortex of rats chronically treated with imipramine: effect of alpha 2-adrenoceptor blockade” Biol Psychiatry. 1999 Sep. 15; 46(6): 766-74.
Lopez, J F, et al., “Regulation of 5-HT receptors and the hypothalamic-pituitary-adrenal axis. Implications for the neurobiology of suicide” Ann NY Acad. Sci. 1997 Dec. 29; 836: 106-34.
Lucki, I, “Behavioral studies of serotonin receptor agonists as antidepressant drugs” J Clin Psychiatry. 1991 December; 52 (Suppl): 24-31.
Malberg, Jessica, et al., “Sustained Use of Anti-Depressants Increases Cell Growth and Protects Cells in the Brain”, 2000 Dec. 15, from: http://www.sciencedaily.com/releases/2000/12/001215081931.htm.
Malison, R T, et al., “Reduced brain serotonin transporter availability in major depression as measured by [1231]-2 beta-carbomethoxy-3 beta-(4-iodophenyl) tropane and single photon emission computed tomography.” 1998 Dec. 1; 44(11): 1090-8.
Marki, A, et al., “Mu-opioid receptor specific antagonist cyprodime: characterization by in vitro radioligand and [³⁵S]GTPgammaS binding assays” Eur J. Pharmacol. 1999 Oct. 27; 383(2): 209-214.
Marriott, I, et al., “IL-4 and IFN-γ Up-Regulate Substance P Receptor Expression in murine Peritoneal Macrophages” J Immunology. 2000; 165: 182-191.
Masand, P S, et al., “Long-term side effects of newer-generaiton antidepressants: SSRIs, venlafaxine, nefazodone, bupropion, and mirtazapine” Ann Clin Psychiatr. 2002 September; 14(3): 175-82.
Mehta, A, et al., “Endomorphin-1: Induction of Motor Behavior and Lack of Receptor Desensitization” J. Neurosci. 2001 Jun. 15; 21(12): 4436-4442.
Meini, S, et al., “GR 73,632 and [Glu(OBzl)11]substance P are selective agonists for the septide-sensitive tachykinin NK1 receptor in the rat urinary bladder” Neuropeptides. 1995 February; 28(2): 99-106.
Millan, M J, et al., “Antagonist-induced opioid receptor up-regulation. I. Characterization of supersensitivity to selective mu and kappa agonists” J Pharmacol Exp Ther. 1988 Nov. 1; 247(2): 721-728.
Minabe, Y, et al., “Effect of the acute and chronic administration of the selective neurokinin2 receptor antagonist SR 48968 on midbrain dopamine neurons in the rat: An in vivo extracellular single cell study” Synapse. 1997; 25: 196-204.
Morris, B J, et al., “Antagonist-induced opioid receptor up-regulation. II. Regionally specific modulation of mu, delta and kappa binding sites in rat brain revealed by quantitative autoradiography” J Pharmacol Exp Ther. 1988 Nov. 1; 247(2): 729-736.
Morteau, O, et al., “Hemokinin 1 is a full agonist at the substance P receptor” Nature Immunology. 2002; 2: 1988.
Murtra, P, et al., “Rewarding effects of opiates are absent in mice lacking the receptor for substance P.” Nature, 2000 May 11; 405: 180-183.
Nakagawa, Y, et al., “The 5-HT3 receptor agonist attenuates the action of antidepressants in the forced swim test in rats” Jpn J. Pharmacol. 1999 July; 80(3): 185-9.
Negus, Stevens, S, et al., “Behavioral Effects of the Delta-Selective Opioid Agonist SNC80 and Related Compounds in Rhesus Monkeys” J Pharmacol Exp Ther. 1998 July; 286(1): 362-375.
Nelson, J C, et al., “Combining norepinephrine and serotonin reuptake inhibition mechanism for treatment of depression: a double-blind, randomized study: Biol Psychiatry. 2004 Feb. 1; 55(3): 296-300.
Nutt, D J, et al., “Mechanism of actions of selective serotonin reuptake inhibitors in the treatment of psychiatric disorders” Eur Neuropsychopharmacol. 1999 July; 9 Suppl 3: S81-6.
O'Neill, W M, et al., “The cognitive and psychomotor effects of morphine in healthy subjects: a randomized controlled trial of repeated (four) oral doses of dextro-propoxyphene, morphine, lorazepam and placebo” Pain. 2000 March; 85(1-2): 209-15.
Pacher, P, et al., “Current trends in the development of new antidepressants” Curr Med. Chem. 2001 February; 8(2): 89-100.
Palma, C, et al., “Substance P activates responses correlated with tumour growth in human glioma cell lines bearing tachykinin NK 1 receptors” Br J Cancer, January 1999; 79(2): 236-43.
Paronis, C A, et al., “Increased analgesic potency of mu agonists after continuous naloxone infusion in rats” J Pharmacol Exp Ther. 1991 Nov. 1; 259(2): 582-589.
Patel, A, et al., “Inhibitors of enkephalin-degrading enzymes as potential therapeutic agents” Prog Med. Chem. 1993; 30: 327-78.
Perez, V, et al., “A Double-blind, Randomized, Placebo-Controlled Trial of Pindolol Augmentation in Depressive Patients Resistant to Serotonin Reuptake Inhibitors” Arch Gen ‘Psychiatry. 1999; 56: 375-379.
Petrillo, P., et al., “Evidence for a Selective Role of the δ-Opioid Agonist ([8R-(4bS*,8aα,8aβ, 12bβ)]7,10-Dimethyl-1-methoxy-11-(2-methylpropyl)oxycarbonyl 5,6,7,8,12,12b-hexahydro-(9H)-4,8-methanobenzofuro[3,2-e]pyrrolo[2,3-g]isoquinoline Hydrochloride (SB235863) in Blocking Hyperalgesia Associated with Inflammatory and Neuropathic Pain Responses” J Pharmacology, First published on 2003 Oct. 9; DOI: 10.1124/jpet.103.055590.
Pomeranz, Bruce, PhD, “Acupuncture and the Raison D'Etre for Alternative Medicine” In: Alternative Therapies in Health and Medicine, 1996 November; 2(6): 85-91.
Porter, S J, et al., “In vivo and in vitro potency studies of 6beta-naltrexol, the major human metabolite of naltrexone” Addict Biol. 2002 April; 7(2): 219-225.
Portoghese, P S, “Molecular recognition at kappa opioid receptors” Pure Appl Chem. 2001; 73(9): 1387-1391.
Puttfarcken, P F, et al., “Effects of chronic morphine exposure on opioid inhibition of adenylyl cyclase in 7315c cell membranes: a useful model for the study of tolerance at mu opioid receptors” Molecular Pharmacol. 1988; 33(5): 520-527.
Ramboz, et al., Proc Natl Acad Sci, USA. 1998; 95: 14480-14481.
Rasmussen, K, et al., “The Novel 5-Hydroxytryptamine_{1A} Antagonist LY 426965: Effects on Nicotine Withdrawal and Interactions with Fluoxetine” J Pharmacol Exp Ther. 2000 August; 294(2): 688-700.
Ripley, T L, et al., “Lack of self-administration and behavioural sensitization to morphine, but not cocaine, in mice lacking NK1 receptors” Neuropharmacol. 2002 December; 43(8): 1258-68.
Roberts, C, et al., “SB-236057-A: A Selective 5-HT(1B) Receptor Inverse Agonist” CNS Drug Rev. 2001 Winter; 7(4): 433-444.
Robinson, D S, et al., “Clinical effects of the 5-HT1A partial agonists in depression: a composite analysis of buspirone in the treatment of depression” J Clin Psychopharmacol. 1990 June; 10(3 Suppl): 65S-76S.
Rogoz, Z, et al., “Effect of repeated treatment with reboxetine on the central alpha1-adrenergic and dopamine D2/D3 receptors” Pol J Pharmacol 2002 November-December; 54(6): 593-603.
Romero, L, et al., “Effect of pindolol on the function of pre-synaptic 5-HT1A receptors: in vivo microdialysis and electrophysiological studies in the rat brain.” Neuropsychopharmacology. 1996 October; 15(4): 349-60.
Ronsisvalle G, et al., “Nonpeptide analogues of dynorphin A(1-8): design, synthesis, and pharmacological evaluation of kappa-selective agonists” J Med. Chem. 2000 Aug. 10; 43(16): 2992-3004
Roth, B L, et al., “Salvinorin A: A potent naturally occurring normitrogenous κ opioid selective agonist” PNAS. 2002 Sep. 3; 99(18): 11934-11939.
Rupniak, N M, “Elucidating the antidepressant actions of substance P (NK1 receptor) antagonists” Pharmacotherapy. 2001 September; 21(9): 1061-9.
Sakurada, C, et al., “Spinal actions of GR73632, a novel tachykinin NK1 receptor agonist” Peptides. 1999; 20(2): 301-4.
Sarnyai, Z, et al., “Impaired hippocampal-dependent learning and funcgtinal abnormalities in the hippocampus in mice lacking serotonin1A receptors” Proc Natl Acad Sci, Neurobiology. 2000 Dec. 19; 97(26): 14731-14736.
Sax, D S, et al., “Lack of hepatotoxicity with naltrexone treatment” J Clin Pharmacol. 1994 September; 34(9): 898-901.
Schatzberg A F, “Clinical efficacy of reboxetine in major depression” J Clin Psychiatry. 2000; 61 Suppl 10: 31-8.
Schindler, C W, et al., “Pharmacological specificity of enhanced sensitivity to naltrexone in rats” Psychopharmacol (Berl). 1993; 110(1-2); 60-8.
Schlicker E, et al., “Interactions between the presynaptic alpha2-autoreceptor and presynaptic inhibitory heteroreceptors on noradrenergic neurone’ Brain Res Bull. 1998 Sep. 15; 47(2): 129-32.
Schmidhammer, H, et al., “Synthesis and biological evaluation of 14-alkoxymorphinans. 3. Extensive study on cyprodime-related compounds” J Med. Chem. 1990 April; 33(4): 1200-6.
Schuh, K J, “Onset, magnitude and duration of opioid blockade produced by buprenorphine and naltrexone in humans” Psychopharmacology (Berl). 1999 July; 145(2); 162-74.
Serres, F, et al., “Coadministration of 5-Hydroxytryptamine_{1A} Antagonist WAY-100635 Prevents Fluoxetine-Induced Desensitization of Postsynaptic 5-Hydroxytryptamine_{1A} Receptors in Hypothalamus” Pharmacology. 2000 July; 294(1): 296-301.
Sheffler, D J, et al., “Salvinorin A: the ‘magic mint’ hallucinogen finds a molecular target in the kappa opioid receptor” TRENDS Pharmacological Sci. 2003 March; 24(3): 107-109.
Shirayama, Y, et al., “Stress increases dynorphin immunoreactivity in limbic brain regions and dynorphin antagonism produces antidepressant-like effects” J Neurochemistry. 2004 September; 90(5): 1258.
Silva, R M, et al., “Dynorphin A₁₋₁₇-Induced Feeding: Pharmacological Characterization Using Selective Opioid Antagonists and Antisense Probes in Rats” J Pharmacol Exp Ther. 2002 May; 301(2): 513-518.
Singh, A, et al., “Antidepressant-like activity of compounds with varying efficacy at 5-HT1A receptors” Neuropharmacology. 1993 April; 32(4): 331-40.
Slassi, A, “Recent Advances in 5-HT1B/1D Receptor Antagonists and Agonists and their Potential Therapeutic Applications” Curr Top Med. Chem. 2002 June; 2(6): 559-74.
Snider, R M, et al., “A potent nonpeptide antagonist of the substance P (NK1) receptor” Science. 1991 Jan. 25; 251(4992): 435-7.
Sofuoglu, M., et al., “Differential antagonism of delta opioid agonists by naltrindole and its benzofuran analog (NTB) in mice; evidence for delta opioid receptor subyypes” J Pharmacol Exp Ther. 1991 May 1; 257(1): 676-680.
Spevak, S E, et al., “The opioid component of the effect of the lacrimal glands on wound healing” Biull Eksp Biol Med. 1987 December; 104(12): 730-3.
Spinks, D., et al., “Serotonin Reuptake Inhibition: An Update on Current Research Strategies” Curr Med. Chem. 2002 April; 9(8): 799-810.
Spreekmeester, E, et al., “Selective mu and delta, but not kappa, opiate receptor antagonists inhibit the habituation of novelty-induced hypoalgesia in the rat” Psychopharmacol (Berl). 2000 January; 148(1): 99-105.
Spooren, W P, et al., “Pharmacological and endocrinological characterization of stress-induced hyperthermia in singly housed mice using classical and candidate anxiolytics (LY314582, MPEP and NKP608)” Eur J. Pharmacol. 2002 Jan. 25; 435(2-3): 161-70.
Sramek, J J, et al., “Establishing the Maximum Tolerated Dose of Lesopitron in Patients with Generalized anxiety Disorder: A Bridging Study” J Clin Psychopharmacol, 1996 December; 16(6): 454-458.
Steinberg, R, et al., “Selective blockade of neurokinin-2 receptors produces antidepressant-like effects associated with reduced corticotropin-releasing factor function” J Pharmacol Exp Ther. 2001 November; 299(2): 449-58.
Stenfors, Carina, “The search of a rapid onset of action through 5-HT presynaptic receptors” Bioscience, Local Discovery, AstraZeneca R&DD Sodertalje, S-15185 Sodertalje, Sweden from: http://www.pasteur.fr/applications/euroconf/depression/stenfors.pdf.
Stockmeier, C A, et al., “Increase in serotonin-1A autoreceptors in the midbrain of suicide victims with major depression—postmortem evidence for decreased serotonin activity” J. Neurosci. 1998 Sep. 15; 18(8): 7394-401.
Suzuki, T., et al., “Opioid receptor types and dependence” Nippon Yakurigaku Zasshi. 1997 April; 109(4): 165-7.
Tejedor-Real, P, et al., “Effect of mixed (RB 38A) and selective (RB 38B) inhibitors of enkephalin degrading enzymes on a model of depression in the rat” Biol Psychiatry. 1993 Jul. 1-15; 34(1-2): 100-7.
Tejedor-Real, P, et al., “Implication of endogenous opioid system in the learned helplessness model of depression” Pharmacol Biochem Behav. 1995 September; 52(1): 145-52.
Tejedor-Real, P., et al., “Involvement of delta-opioid receptors in the effects induced by endogenous enkephalins on learned helplessness model.” Eur J. Pharmacol. 1998 Jul. 31: 354(1): 1-7.
Tempel, A, et al., “Neurochemical and functional correlates of naltrexone-induced opiate receptor up-regulation” J Pharmacol Exp Ther. 1985 Feb. 1; 232(2): 439-444.
Todtenkopf, M F, et al., “Effects of kappa-opioid receptor ligands on intracranial self-stimulation in rats” Psychopharmacol (Berl). 2004 April; 172(4): 463-70.
Tome, M B, et al., “Paroxetine and pindolol: a randomized trial of serotonergic autoreceptor blockade in the reduction of antidepressant latency.” Int Clin Psychopharmacol. 1997 March; 12(2): 81-9.
Tottori, K, et al., “Antidepressant-like responses to the combined sigma and 5-HT1A receptor agonist OPC-14523” Neuropharmacol. 2001 December; 41(8): 976-988.
Valles, J, et al., “Oral idazoxan bioavailability in rat. Relevance of intestinal and hepatic first-pass effect.” Drug Metab Dispos. 1989 November-December; 17(6): 673-6.
Vanderah, T, et al., “Dynorphin Promotes Abnormal Pain and Spinal Opioid Antinociceptive Tolerance” J. Neurosci. 2000 Sep. 15; 20(18): 7074-7079.
Van Schoor, J., et al., “The effect of the NK2 tachykinin receptor antagonist SR 48968 (saredutant) on neurokinin A-induced bronchoconstriction in asthmatics” Eur Respir J, 1998; 12: 17-23.
Varty, G B, et al., “The Geril Elevated Plus-maze II: Anxiolytic-like Effects of Selective Neurokinin NK1 Receptor Antagonists” Neuropsychopharmacology 2002; 27: 371-379.
Vinogradov, V A., et al., “Opioid activity of peptides and wound healing of the skin” [Article in Russian] Biull Eksp Biol Med. 1987 July; 104(7): 89-91
Wang, Y, et al., “Comparison of Pharmacological Activities of Three Distinct κ-ligands (Salvinorin A, TRK-820 and 3 FLB) on κOpioid Receptors in vitro and their Antipruritic and Antinociceptive Activities in vivo” J Pharmacol Exp Ther. 2004 Sep. 21; 10.1124
Wang, Z, et al., “Pronociceptive Actions of Dynorphin Maintain Chronic Neuropathic Pain” J. Neurosci. 2001 March; 21(5): 1779-1786.
Wen, H L, et al., “Treatment of pain in cancer patients by intrathecal administration of dynorphin” Peptides. 1987 January-February; 8(1): 191-3.
Wybran, J, “Enkephalins and endorphins as modifiers of the immune system: present and future.” Fed Proc. 1985 January; 44(1 Pt 1): 92-4.
Xu, M, et al., “Kappa opioid receptor activation in the spinal cord dorsal horn following partial sciatic nerve ligation in mice” Annual Meeting, American Pain Society, B. Systems (Physiology, Anatomy, Animal Models) B 17-Spinal Nociceptive Mechanisms, Poster #695.
Yamamoto, T, et al., REGIONAL ANESTHESIA AND PAIN MANAGEMENT, “The Role of Spinal Opioid Receptor Like 1 Receptor, the NK-1 Receptor, and Cyclooxygenase-2 in Maintaining Postoperative Pain in the Rat” Anesth Analg. 1999; 89: 120
Yasuno, F, et al., “Inhibitory Effect of Hippocampal 5-HT1A Receptors on Human Explicit Memory” Am J Psychiatry. 2003 February; 160: 334-340.
Ybema, C E, et al., “Discriminative stimulus effect of flesinoxan: effect of 5-HT1A antagonists and PCPA” Pharmacol Biochem Behav. 1994 April; 47(4): 957-62.
Yilmaz, B, et al., “Delta opioid modulation of hypothalamic serotonergic neurotransmission in the ovariectomized and steroid-primed rat” Neuroendocrinology Letters. 2000; 21(2): 115-120.
Zadina, J E, et al., “Mu opiate receptor down-regulation by morphine and up-regulation by naloxone in SH-SY5Y human neuroblastoma cells” J Pharmacol Exp Ther. 1993 April; 265(1): 254-262.
Zadina, J E, et al., “Differential regulation of mu and delta opiate receptors by morphine, selective agonists and antagonists and differentiating agents in SH-SY5Y human neuroblastoma cells” J Pharmacol Exp Ther. 1994 September; 270(3): 1086-96.
Zernig, G, et al., “In vivo determination of mu opioid receptor turnover in rhesus monkeys after irreversible blockade with clocinnamox” J Pharmacol Exp Ther. 1994 Apr. 1; 269(1) 57-65.
Zurita, A, et al., “An endogenous opiate mechanism seems to be involved in stress-induced anhedonia” Eur J. Pharmacol. 1996 Mar. 28; 299(1-3): 1-7.

What is claimed is:

1. A method of regulating a neurotransmitter system by inducing a counteradaptation in a patient, the neurotransmitter system including a type of receptor linked to an undesirable mental or neurological condition, the method comprising the step of:
repeatedly administering to the patient a ligand for the type of receptor, each administration having an administration half-life, thereby causing the ligand to bind receptors of that type during a first time period associated with each administration, thereby inducing a counteradaptation,
wherein the counteradaptation causes the regulation of the neurotransmitter system, and
wherein the ratio of the administration half-life to the period between administrations is no greater than 1/2.

2. A method of inducing a regulation of a neurotransmitter system in a patient, the neurotransmitter system including a type of receptor linked to an undesirable mental or neurological condition, the method comprising the steps of:
inducing a counteradaptation by giving the patient a ligand for the type of receptor; then
repeatedly administering to the patient a ligand for the type of receptor, each administration having an administration half-life, thereby causing the ligand to bind receptors of that type during a first time period associated with each administration, thereby maintaining or improving the counteradaptation,
wherein the counteradaptation causes the regulation of the neurotransmitter system, and
wherein the ratio of the administration half-life to the period between administrations is no greater than 1/2.

3. The method of claim 1 or claim 2, wherein the regulation of the neurotransmitter system causes a therapeutic benefit with respect to the undesirable mental or neurological condition.

4. The method of any one of claims 1-3, wherein the undesirable mental or neurological condition is positively linked to the type of receptor, the ligand is a receptor agonist and the regulation is a down-regulation of the neurotransmitter system.

5. The method of claim 4, wherein the counteradaptation is at least one of:
a decrease in the biosynthesis or release of a neurotransmitter binding to receptors of the type of receptor;
an increase in the reuptake of a neurotransmitter binding to receptors of the type of receptor;
a decrease in the number of the type of receptors and/or binding sites on receptors of the type of receptor; and
a decrease in the sensitivity of receptors of the type of receptor to binding by natural neurotransmitter and/or receptor agonists.

6. The method of any one of claims 4-5, wherein an antagonist for the type of receptor is not administered during the first time period associated with each administration.

7. The method of any one of claims 4-6, wherein each administration has a second time period associated therewith, the second time period being subsequent to the first time period associated with the administration, and wherein an antagonist for the type of receptor is administered during one or more of the second time periods.

8. The method of any one of claims 1-3, wherein if the undesirable mental or neurological condition is negatively linked to the type of receptor, the ligand is an antagonist and the regulation is an up-regulation of the neurotransmitter system.

9. The method of claim 8, wherein the counteradaptation is at least one of:
an increase in the biosynthesis or release of a neurotransmitter binding to receptors of the type of receptor;
a decrease in the reuptake of a neurotransmitter binding to receptors of the type of receptor;
an increase in the number of the type of receptors and/or binding sites on receptors of the type of receptors; and
an increase in the sensitivity of the receptors to binding by ligands and/or receptor agonists.

10. The method of any one of claims 8-9, wherein an agonist for the type of receptor is not administered during the first time period associated with each administration.

11. The method of any one of claims 8-10, wherein each administration has a second time period associated therewith, the second time period being subsequent to the first time period associated with the administration, and wherein an agonist for the type of receptor is administered during one or more of the second time periods.

12. The method of any one of claims 1-11, wherein a substantial fraction of the receptors of the type of receptor are bound by the ligand during each first time period.

13. The method of claim 12, wherein at least about 30%, at least about 50%, at least about 75% or at least about 90% of the receptors are bound by the ligand during each first time period.

14. The method of any one of claims 1-13, wherein each first time period is at least about five minutes in duration; at least about thirty minutes in duration; at least about an hour in duration; at least about two hours in duration; or at least about four hours in duration.

15. The method of any one of claims 1-14, wherein each first time period is less than about twenty four hours in duration; less than about sixteen hours in duration; less than about twelve hours in duration; less than about eight hours in duration; or less than about six hours in duration.

16. The method of any one of claims 1-16, wherein each administration has a second time period associated therewith, the second time period being subsequent to the first time period associated with the administration, and wherein a substantial fraction of the receptors remain unbound to the ligand during each second time period.

17. The method of claim 16, wherein no more than about 50%, no more than about 25%, or no more than about 10% of the receptors are bound to the ligand during each second time period.

18. The method of any one of claims 7, 11 and 16-17, wherein each second time period is at least about two hours in duration; at least about ten hours in duration; or at least about fifteen hours in duration.

19. The method of any one of claims 7, 11 and 16-18, wherein each second time period is no more than about twenty hours in duration; no more than about thirty hours in duration; or no more than about fifty hours in duration.

20. The method of any one of claims 1-19, wherein the ratio of the administration half-life to the period between administrations is no greater than 1/3.

21. The method of any one of claims 1-20, wherein the ratio of the administration half-life of the ligand to the period between administrations is no greater than 1/5; no greater than 1/8; or no greater than 1/12.

22. The method of any one of claims 1-21, wherein the ratio of the administration half-life of the ligand to the period between administrations is greater than 1/24; greater than 1/12; greater than 1/8; greater than 1/5; greater than 1/4; or greater than 1/3.

23. The method of any one of claims 1-22, wherein the dose of ligand at each administration is increased over time.

24. The method of any one of claims 1-23, wherein the dose of ligand at each administration is increased intermittently over time.

25. The method of any one of claims 1-24, wherein the dose is increased with a period between increases of no less than a week; no less than two weeks; no less than three weeks; no less than a month; no less than two months; no less than three months; no less than six months, or no less than one year.

26. The method of any one of claims 1-25, wherein at each increase in dosage, the dose is increased by at least 5%; at least 10%; at least 25%; at least 50%; or at least 100% of the initial dose.

27. The method of any one of claims 1-26, wherein the maximum dosage is within three hundred times the initial dosage, within one hundred times the initial dosage, within fifty times the initial dosage, or within twenty times the initial dosage.

28. The method of any one of claims 1-27, wherein the administration of the ligand is performed daily.

29. The method of any one of claims 1-28, wherein the period between administrations is two days or greater; three days or greater; five days or greater; one week or greater; two weeks or greater; or one month or greater.

30. The method of any one of claims 1-29, wherein the administration half-life is less than about sixteen hours; less than about twelve hours; less than about eight hours; or less than about four hours.

31. The method of any one of claims 1-30, wherein the administration half-life is greater than about four hours; greater than about twelve hours; greater than about sixteen hours; or
greater than about thirty hours.

32. The method of any one of claims 1-31, wherein the compound half-life of the ligand is less than about sixteen hours; less than about twelve hours; less than about eight hours; or less than about four hours.

33. The method of any one of claims 1-32, wherein the compound half-life of the ligand is greater than about four hours; greater than about twelve hours; greater than about sixteen hours; or greater than about thirty hours.

34. The method of claim 33, wherein the compound half-life of the ligand is greater than about twelve hours, and wherein the method further comprises the step of
administering repeatedly and with a period of less than every two days a second ligand for the type of receptor, each administration of the second ligand having an administration half-life of less than about eight hours.

35. The method of claim 34, wherein the ligand having the compound half-life greater than about twelve hours is an agonist, and the second ligand is an agonist.

36. The method of claim 34, wherein the ligand having the compound half-life greater than about twelve hours is an antagonist, and the second ligand is an antagonist.

37. The method of any one of claims 1-36, wherein the administration is repeated at least five times, at least ten times, at least twenty-five times, or at least fifty times.

38. The method of any one of claims 1-37, wherein the dose of the ligand is sufficient to trigger a counteradaptive response, but low enough that direct effects of ligand-receptor binding are low and tolerable to the patient.

39. The method of any one of claims 1-38, wherein a substantial fraction of the first time period occurs while the patient is asleep.

40. The method of any one of claims 1-39, wherein at least 40%; at least 60%; or at least 85% of the first time period occurs while the patient is asleep.

41. The method of any one of claims 1-40, wherein each administration of the ligand is performed within the hour before the patient goes to bed.

42. The method of claim 1-40, wherein each administration of the ligand is performed more than one hour before the patient goes to bed.

43. The method of claim 1-42, wherein each administration of the ligand is performed orally, transdermally, through inhalation, subcutaneously, intravenously, intramuscularly, intraspinally, intrathecally, transmucosally, or using an osmotic pump, a microcapsule, an implant or a suspension.

44. The method of any one of claims 1-43, further comprising the step of:
administering an anxiolytic agent in combination with the ligand.

45. The method of claim 44, wherein the anxiolytic agent affects a GABA pathway.

46. The method of claim 44, wherein the anxiolytic agent is a benzodiazepine.

47. The method of claim 46, wherein the benzodiazepine is selected from the group consisting of diazepam, lorazepam, alprazolam, temazepam, flurazepam, and chlodiazepoxide.

48. The method of any one of claims 1-47, further comprising the step of:
administering a hypnotic agent in combination with the ligand.

49. The method of any one of claims 1-48, further comprising the step of:
administering in combination with the ligand a TCA, an MAOI, an SSRI, an NRI, an SNRI, a CRF modulating agent, a serotonin pre-synaptic autoreceptor antagonist, 5HT₁ agonist, a dynorphin antagonist, a GABA-A modulating agent, a serotonin 5H_{2C} and/or 5H_{2B} modulating agent, a beta-3 adrenoceptor agonist, an NMDA antagonist, a V1B antagonist, a GPCR modulating agent, or a substance P antagonist.

50. The method according to claim 49 wherein SSR1 is selected from the group consisting of fluoxetine (Prozac®), paroxetine (Paxil®), sertraline (Zoloft®), fluvoxamine (Luvox®), citalopram (Celexa®), escitalopram (Lexapro®); SNRIs are selected from the group consisting of venlafaxine (Effexor®) and mefazodone (Serzone®); and NRIs comprise reboxetine (edronax®).

51. The method of any one of claims 1-49, wherein the method is used to address the undesirable mental or neurological condition in a patient.

52. The method of any one of claims 1-51, wherein
the neurotransmitter system is the SP system;
the type of receptor is SP receptors;
the ligand is an SP receptor agonist;
the undesirable mental or neurological condition is positively linked to the receptors; and
the counteradaptation causes a down-regulation of the SP system.

53. The method of claim 52, wherein the counteradaptation is at least one of:
a decrease in the biosynthesis or release of SP, NKA, and/or NKB at the receptor terminals or by the pituitary gland;
a decrease in the number of the receptors and/or binding sites on the receptors; and
a decrease in the sensitivity of the receptors to binding by SP receptor agonists and/or SP, NKA, and/or NKB.

54. The method of any one of claims 52-53, wherein the SP receptor agonist is peptide-based.

55. The method of any one of claims 52-53, wherein the SP receptor agonist is an analogue of SP, NKA, and/or NKB, or a pharmaceutically-accepted salt or derivative thereof.

56. The method of any one of claims 52-53, wherein the SP receptor agonist is Substance P; Substance P, free acid; Biotin-Substance P; [Cys_{3,6}, Tyr₈, Pro₉]-Substance P; (Disulfide bridge: 3-6), [Cys_{3,6}, Tyr₈, Pro₁₀]-Substance P; (Disulfide bridge: 3-6), [4-Chloro-Phe_{7,8}]-Substance P; [4-Benzoyl-Phe₈]-Substance P; [Succinyl-Asp₆, N-Me-Phe₈]-Substance P (6-11)(Senktide); [Tyr₈]-Substance P; [Tyr₈]-Substance P; or Shark Substance P Peptide.

57. The method of any one of claims 52-53, wherein the SP receptor agonist is an NKA or NKB analogue having a C-terminal heptapeptide similar to NKA(4-10) or NKB (4-10), or a pharmaceutically acceptable salt or carrier thereof.

58. The method of any one of claims 52-53, wherein the SP receptor agonist is [Gln⁴]-NKA, [GlN⁴]-NKA(4-10), [Phe⁷]-NKA, [Phe⁷]-NKA(4-10), [Ile⁷]-NKA, [Ile7]-NKA(4-10), [Lys⁵,MeLeu⁹,Nle¹⁰]-NKA(4-10), [Nle¹⁰]-NKA(4-10), β-Ala8]-NKA(4-10), [Ala⁵]-NKA(4-10), [Gln⁴]-NKB, [GlN⁴]-NKB(4-10), [Phe⁷]-NKB, [Phe⁷]-NKB(4-10), [Ile⁷]-NKB, [Ile7]-NKB(4-10), [Lys⁵,MeLeu⁹,Nle¹⁰]-NKB(4-10), [Nle¹⁰]-NKB(4-10), β-Ala8]-NKB(4-10), [Ala⁵]-NKB(4-10), GR 73,632 [delta-Aminovaleryl [Pro9, N-Me-Leu10]-substance P(7-11)], [Glu(OBzl)11]substance P and hemokinin 1 (HK-1) (a substance P homolog) or a pharmaceutically acceptable salt or carrier thereof.

59. The method of any one of claims 52-53, wherein the SP receptor agonist is [Arg]-NKB or a pharmaceutically acceptable salt or carrier thereof.

60. The method of any one of claims 52-53, wherein the SP receptor agonist is an NKA or NKB analogue having Val⁷ replaced with MePhe or a pharmaceutically acceptable salt or carrier thereof.

61. The method of any one of claims 52-60, wherein the initial dosage of the SP receptor agonist is between about 0.1 to 100 ug/kg/day initial dose and 100 to 1000 ug/kg/day for 8 hours slow release.

62. The method of any one of claims 52-60, wherein the initial dosage of the SP receptor agonist is between about 1 to 50 ug/kg/day initial dose and 20 to 50 ug/kg/day for 8 hours slow release.

63. The method of any one of claims 52-62, wherein the undesirable mental or neurological condition is chronic pain, a mood disorder, an eating disorder, an anxiety disorder, a motivational problem, a substance abuse disorder, an inflammatory condition, nausea or emesis, urinary incontinence, skin rashes, erythema, or eruptions.

64. The method of any one of claims 52-62, wherein the undesirable mental or neurological condition is fibromyalgia, chronic fatigue syndrome, chronic back pain, chronic headaches, chronic cancer pain, shingles, reflex sympathetic dystrophy, neuropathy, or inflammatory pain.

65. The method of any one of claims 52-62, wherein the undesirable mental or neurological condition is pain that is anticipated to occur in the future.

66. The method of claim 65, wherein the pain anticipated to occur in the future is pain from a medical procedure, or pain due to physical exertion.

67. The method of any one of claims 52-62 wherein the undesirable mental or neurological condition is a major depressive disorder, post-traumatic depression, temporary depressed mood, manic-depressive disorder, dysthymic disorder, generalized mood disorder, anhedonia, or non-organic sexual disfunction.

68. The method of any one of claims 52-62, wherein the undesirable mental or neurological condition is overeating, obesity, anorexia or bulimia.

69. The method of any one of claims 52-62, wherein the undesirable mental or neurological condition is a generalized anxiety state, a panic disorder, a phobia, obsessive-compulsive disorder, attention deficit hyperactivity disorder, Tourette's Syndrome, a hysteria sleep disorder, or a breathing-related sleep disorder.

70. The method of any one of claims 52-62, wherein the undesirable mental or neurological condition is a lack of motivation due to learning or memory problems.

71. The method of any one of claims 52-62, wherein the undesirable mental or neurological condition is abuse of a substance selected from the group consisting of narcotics, alcohol, nicotine, stimulants, anxiolytics, CNS depressants, hallucinogens and marijuana.

72. The method of any one of claims 52-62, wherein the undesirable mental or neurological condition is asthma, arthritis, rhinitis, conjunctivitis, inflammatory bowel disease, inflammation of the skin or mucosa, or acute pancreatitis.

73. The method of any one of claims 52-62, wherein the undesirable mental or neurological condition is nausea or emesis arising from chemotherapy.

74. The method of any one of claims 52-73, wherein the method is used to address the undesirable mental or neurological condition in a patient.

75. The method of any one of claims 52-74, wherein the method is used as an adjunct treatment for cancer.

76. The method of any one of claims 52-75, wherein SP receptor antagonist is not administered during the first time period associated with each administration.

77. The method of any one of claims 52-76, wherein an SP receptor antagonist is administered during one or more of the second time periods.

78. The method of any one of claims 52-76, wherein the SP receptor antagonist is L-760735, CP-96,345, NKP608, L-AT, MK-869, L-742,694, L-733060, CP-99,994, P-122,721, CP 122,171, GSK 597599, GSK 679769, GSK 823296, Saredutant, Talnetant, Osanetant, and pharmaceutically acceptable salts, analogues, and derivatives thereof.

79. The method of claim 78, wherein initial dosage of SP receptor antagonist is equivalent to 12 mg/kg/hour for 8 hours of L-760735; about 30 ug/kg/hour of CP-96,345; between 0.1 to 10 mg/kg/administration of SSR240600; between 0.01 to 0.1 mg/kg/administration of NKP608 (via po); between 1 to 10 mg/kg/administration of L-AT; between 0.01 to 3 mg/kg/administration of MK-869; between 1 to 30 mg/kg of L-742,694; between 1 to 10 mg/kg/administration of L-733,060; between 3 to 30 mg/kg/administration of CP-99,994 or CP-122,721; and about 100 mg/administration of Saredutant.

80. The method of any one of claims 51-79, wherein the down-regulation of the SP system causes a therapeutic benefit with respect to the undesirable mental or neurological condition.

81. The method of any one of claims 1-51, wherein
the neurotransmitter system is the endogenous endorphin system;
the type of receptor is mu and/or delta opiate receptors;
the ligand is a mu and/or delta opiate receptor antagonist;
the undesirable mental or neurological condition is negatively linked to the receptors; and
the counteradaptation causes an up-regulation of the endogenous endorphin system.

82. The method of claim 81, wherein the counteradaptation is at least one of:
an increase in the biosynthesis or release of endorphins at receptor terminals and/or by the pituitary gland;
an increase in the number of the receptors and/or endorphin binding sites on the receptors; and
an increase in the sensitivity of the receptors to binding by mu and/or delta opiate agonists and/or endorphins.

83. The method of any one of claims 81-82, wherein the counteradaptation is at least one of:
an increase in the biosynthesis or release of endorphins at receptor terminals and by the pituitary gland; and
an increase in the number of the receptors and/or endorphin binding sites on the receptors.

84. The method of any one of claims 81-83, wherein the mu and/or delta opiate receptor antagonist is a specific mu receptor antagonist or a specific delta receptor antagonist.

85. The method of any one of claims 81-84, wherein the mu and/or delta opiate receptor antagonist is a specific mu opiate receptor antagonist selected from the group consisting of clocinnamox mesylate, CTAP, CTOP, etonitazenyl isothiocyanate, β-funaltrexamine hydrochloride, naloxonazine dihydrochloride, Cyprodime, and pharmaceutically acceptable salts, analogues, and derivatives thereof.

86. The method of any one of claims 81-84, wherein the mu and/or delta opiate receptor antagonist is a specific delta opiate receptor antagonist selected from the group consisting of naltrindole, N-benzylnaltrindole HCl, BNTX maleate, ICI-154,129, ICI-174,864, naltriben mesylate, SDM25N HCl, 7-benzylidenenaltrexone, and pharmaceutically acceptable salts, analogues, and derivatives thereof.

87. The method of any one of claims 81-83, wherein the mu and/or delta opiate receptor antagonist is a non-specific opiate antagonist.

88. The method of any one of claims 81-83, wherein the mu and/or delta opiate receptor antagonist is naloxone, naltrexone, nalmefene, or nalbuphine, or a pharmaceutically acceptable salt or derivative thereof.

89. The method of any one of claims 81-88, wherein the initial dosage of the mu and/or delta opiate receptor antagonist is equivalent to between about 2 mg/administration and about 200 mg/administration of naloxone.

90. The method of any one of claims 81-88, wherein the initial dosage of the mu and/or delta opiate receptor antagonist is equivalent to between about 10 mg/administration and about 100 mg/administration of naloxone.

91. The method of any one of claims 81-90, wherein the mu and/or delta opiate receptor antagonist is naloxone.

92. The method of claim 91 wherein each dosage of naloxone is greater than 10 mg/administration; greater than 10.5 mg/administration; greater than 11 mg/administration;
or greater than 15 mg/administration.

93. The method of any one of claims 91-92, wherein the initial dosage of naloxone is between 10 and 50 mg/administration.

94. The method of any one of claims 91-92, wherein the initial dosage of naloxone is between 5 and 500 mg/administration.

95. The method of any one of claims 91-94, wherein the maximum dosage of naloxone is no greater than 3000 mg/administration.

96. The method of any one of claims 81-95, wherein the mu and/or delta opiate receptor antagonist is administered using a time-release or slow-release formulation.

97. The method of any one of claims 81-95, wherein the mu and/or delta opiate receptor antagonist is administered orally, transdermally, intraspinally, intrathecally, via inhalation, subcutaneously, intravenously, intramuscularly, or transmucosally, or via osmotic pump, microcapsule, implant, or suspension.

98. The method of any one of claims 81-95, wherein the mu and/or delta opiate receptor antagonist is administered transdermally.

99. The method of any one of claims 96-98, wherein the mu and/or delta opiate receptor antagonist is released over a time period between 2 and 12 hours in duration; between 2 and 6 hours in duration; or between 6 and 12 hours in duration.

100. The method of any one of claims 81-95, wherein the mu and/or delta opiate receptor antagonist is administered as a rapidly absorbed loading dose.

101. The method of any one of claims 81-95, wherein the mu and/or delta opiate receptor antagonist is administered using both a rapidly absorbed loading dose, and transdermal administration or a time-release or slow-release formulation.

102. The method of any one of claims 81-101, wherein a specific mu and/or delta receptor antagonist and a non-specific mu and/or delta receptor antagonist are administered substantially simultaneously.

103. The method of any one of claims 81-101, wherein a specific mu and/or delta receptor antagonist and a non-specific mu and/or delta receptor antagonist are administered sequentially.

104. The method of any one of claims 81-103, wherein the undesirable mental or neurological condition is pain, a mood disorder, an eating disorder, an anxiety disorder, a motivational problem, a substance abuse disorder, insufficient motivation or performance, an immune system-related condition, and a wound in need of healing.

105. The method of any one of claims 81-103, wherein the undesirable mental or neurological condition is pain that is expected to occur in the future, a chronic pain syndrome, or acute pain.

106. The method of any one of claims 81-103, wherein the undesirable mental or neurological condition is pain expected to occur due to a future operation, pain expected to occur due to future physical exertion, fibromyalgia, chronic fatigue syndrome, chronic back pain, chronic headaches, shingles, reflex sympathetic dystrophy, neuropathy, inflammatory pain, or chronic cancer pain.

107. The method of any one of claims 81-103, wherein the undesirable mental or neurological condition is a major depressive disorder, post traumatic depression, a temporary depressed mood, a manic-depressive disorder, a dysthymic disorder, a generalized mood disorder, anhedonia, or non-organic sexual dysfunction.

108. The method of any one of claims 81-103, wherein the undesirable mental or neurological condition is overeating, obesity, anorexia, or bulimia.

109. The method of any one of claims 81-103, wherein the undesirable mental or neurological condition is a generalized anxiety state, a panic disorder, Tourette's Syndrome, a hysteria sleep disorder, or a breathing-related sleep disorder.

110. The method of any one of claims 81-103, wherein the undesirable mental or neurological condition is a lack of motivation due to learning or memory problems.

111. The method of any one of claims 81-103, wherein the undesirable mental or neurological condition is abuse of a substance selected from the group consisting of narcotics, alcohol, nicotine, stimulants, anxiolytics, CNS depressants, hallucinogens and marijuana.

112. The method of any one of claims 81-103, wherein the undesirable mental or neurological condition is insufficient motivation for a desired mental or physical activity.

113. The method of claim 112, wherein the desired activity is physical training, athletics, studying, or testing.

114. The method of any one of claims 81-113, wherein the method is used to address the undesirable mental or neurological condition in a patient.

115. The method of any one of claims 81-114, wherein the method is used as an adjunct treatment for cancer, infection, AIDS, or a wound.

116. The method of any one of 81-114, wherein a mu and/or delta opiate receptor agonist is not administered during the first time period associated with each administration.

117. The method of any one of claims 81-116, wherein a mu and/or delta opiate receptor agonist is administered during one or more of the second time periods.

118. The method of any one of claims 81-116, wherein the up-regulation of the endogenous endorphin system causes a therapeutic benefit with respect to the undesirable mental or neurological condition.

119. The method of any one of claims 1-51, wherein
the neurotransmitter system is the dynorphin system;
the type of receptor is kappa receptors;
the ligand is a kappa receptor agonist;
the undesirable mental or neurological condition is positively linked to the receptors; and
the counteradaptation causes a down-regulation of the dynorphin system.

120. The method of claim 119, wherein the counteradaptation is at least one of:
a decrease in the biosynthesis or release of dynorphins at the receptor terminals or by the pituitary gland;
a decrease in the number of the kappa receptors and/or binding sites on the kappa receptors; and
a decrease in the sensitivity of the kappa receptors to binding by dappa receptor agonists and/or dynorphins.

121. The method of any one of claims 119-120, wherein the kappa receptor agonist is peptide-based.

122. The method of any one of claims 119-120, wherein the kappa receptor agonist is dynorphin or a pharmaceutically acceptable salt, carrier, or analogue thereof.

123. The method of any one of claims 119-120, wherein the kappa receptor agonist is a nonbenzomorphan; enadoline; PD117302; CAM569; PD123497; GR 89,696; U69,593; TRK-820; trans-3,4-dichloro-N-methyl-N-[1-(1-pyrrolidinyl)cyclohexyl]benzene-acetamide; asimadoline (EMD-61753); benzeneacetamide; thiomorpholine; piperidine; benzo[b]thiophene-4-acetamide; trans-(+/−)-(PD-117302); 4-benzofuranacetamide (PD-129190); 2,6-methano-3-bezazocin-8-ol (MR-1268); morphinan-3-ol (KT-90); GR-45809; 1-piperazinecarboxylic acid (GR-89696); GR-103545; piperzaine; GR-94839; xorphanl; benzeneacetamide (RU-49679); fedotozine; benzeneacetamide (DuP-747); HN-11608; apadoline (RP-60180); spiradoline mesylate; benzeneacetamide trans-U-50488 methane sulfate; 3FLB; FE200665; FE200666; an analogue of MPCB-GRR1 or MPCB-RR1, an analogue of the C-terminal fragment of dynorphin A(1-8), or a pharmaceutically-accepted salt or carrier thereof.

124. The method of any one of claims 119-123, wherein the initial dosage of the kappa receptor agonist is equivalent to between 0.0005 and 0.05 mg/kg/administration of dynorphin; between 5 and 700 mg/administration of enadoline; between 1 and 500 μg/administration of FE 20665; between 0.5 and 100 μg/administration; between 0.01 and 1 mg/kg/administration of U69,593; between 0.05 and 5 mg/kg/administration of TRK 820; between 0.01 and 1 mg/kg/administration U 50 488; or between 0.01 and 1 mg/kg/administration of PD 117302.

125. The method of any one of claims 119-123, wherein the initial dosage of the kappa receptor agonist is equivalent to between 0.005 and 0.02 mg/kg/administration of dynorphin; between 100 and 500 mg/administration of enadoline; between 3 and 100 μg/administration of FE 20665; between 1 and 80 μg/administration of FE 20666; between 0.1 and 0.7 mg/kg/administration of U69,593; between 0.5 and 3 mg/kg/administration of TRK 820; between 0.5 and 7 mg/kg/administration U 50 488 or between 0.1 and 0.7 mg/kg/administration of PD 117302.

126. The method of any one of claims 119-123, wherein the kappa receptor agonist is Salvinorin A.

127. The method of claim 126, wherein the initial dose of Salvinorin A is between 5 and 200 ug/administration.

128. The method of any one of claims 126-127, wherein the maximum dose of Salvinorum A is at least 5000 ug/administration.

129. The method of any one of claims 126-128, wherein the Salvinorin A is administered transmucosally.

130. The method of any one of claims 126-129, wherein the Salvinorum A is administered as a slow-release formulation.

131. The method of claim 130, wherein the Salvinorum A is administered over a period two to six hours in duration.

132. The method of any one of claims 119-131, wherein a peptide-based kappa receptor agonist and a non-peptide-based kappa receptor agonist are administered substantially simultaneously.

133. The method of any one of claims 119-131, wherein a peptide-based kappa receptor agonist and a non-peptide-based kappa receptor agonist are administered sequentially.

134. The method of any one of claims 119-133, wherein the condition is pain, a mood disorder, an eating disorder, an anxiety disorder, a motivational problem, a substance abuse disorder, or insufficient motivation or performance.

135. The method of any one of claims 119-133, wherein the condition is pain that is expected to occur in the future; a chronic pain syndrome; or acute pain.

136. The method of any one of claims 119-133, wherein the condition is pain expected to occur due to a future operation; pain expected to occur due to future physical exertion; fibromyalgia; chronic fatigue syndrome; chronic back pain; chronic headaches; shingles; reflex sympathetic dystrophy; neuropathy; inflammatory pain; or chronic cancer pain.

137. The method of any one of claims 119-133, wherein the condition is a major depressive disorder; post traumatic depression; a temporary depressed mood; a manic-depressive disorder; a dysthymic disorder; a generalized mood disorder; anhedonia; or non-organic sexual dysfunction.

138. The method of any one of claims 119-133, wherein the condition is overeating; obesity; anorexia; or bulimia.

139. The method of any one of claims 119-133, wherein the condition is a generalized anxiety state; a panic disorder; Tourette's Syndrome; a hysteria sleep disorder; or a breathing-related sleep disorder.

140. The method of any one of claims 119-133, wherein the condition is a lack of motivation due to learning or memory problems.

141. The method of any one of claims 119-133, wherein the condition is abuse of a substance selected from the group consisting of narcotics, alcohol, nicotine, stimulants, anxiolytics, CNS depressants, hallucinogens and marijuana.

142. The method of any one of claims 119-133, wherein the condition is insufficient motivation for a desired mental or physical activity.

143. The method of claim 142, wherein the desired activity is physical training; athletics; studying; or testing.

144. The method of any one of claims 119-143, wherein the method is used to address the undesirable mental or neurological condition in a patient.

145. The method of any one of claims 119-144, wherein the method is used as an adjunct treatment for cancer.

146. The method of any one of claims 119-145, wherein kappa receptor antagonist is not administered during the first time period associated with each administration.

147. The method of any one of claims 119-146, wherein a kappa receptor antagonist is administered during one or more of the second time periods.

148. The method of any one of claims 119-147, wherein the down-regulation of the dynorphin system causes a therapeutic benefit with respect to the undesirable mental or neurological condition.

149. The method of any one of claims 1-51, wherein
the neurotransmitter system is the serotonin system; and
the counteradaptation causes an up-regulation of the serotonin system.

150. The method of claim 149, wherein
the type of receptor is serotonin pre-synaptic autoreceptors;
the ligand is a serotonin pre-synaptic autoreceptor agonist; and
the undesirable mental or neurological condition is positively linked to the serotonin pre-synaptic autoreceptors.

151. The method of claim 150, wherein the counteradaptation is at least one of:
an increase in the biosynthesis and/or release of serotonin at the synaptic cleft;
a decrease in the reuptake of serotonin;
a decrease in the number of serotonin pre-synaptic autoreceptors;
a decrease in the sensitivity of the serotonin pre-synaptic autoreceptors to serotonin and/or serotonin pre-synaptic autoreceptor agonists;
an increase in the number of serotonin post-synaptic receptors; or
an increase in the sensitivity of the serotonin post-synaptic receptors to serotonin or serotonin post-synaptic receptor agonists.

152. The method of any one of claims 150-151, wherein the serotonin pre-synapatic autoreceptors are 5HT_{1A} autoreceptors and/or 5HT_{1B} autoreceptors.

153. The method of any one of claims 150-152, wherein the serotonin pre-synaptic autoreceptor agonist is EMD-68843, buspirone, gepirone, ipsapirone, tandospirone, Lesopitron, zalospirone, MDL-73005EF, or BP-554.

154. The method of any one of claims 150-153, wherein the initial dosage of the serotonin pre-synaptic autoreceptor agonist is equivalent to between 1 and 400 mg/administration of EMD-68843, between 1 and 500 mg/administration buspirone between 1 and 500 mg/administration lesopitron, between 1 and 500 mg/administration gepirone, between 5 and 500 mg tandospirone, or between 1 and 200 mg zalospirone.

155. The method of any one of claims 150-154, wherein the initial dosage of the serotonin pre-synaptic autoreceptor agonist is equivalent to between 10 and 100 mg/administration of EMD-68843, between 10 and 100 mg/administration buspirone between 10 and 200 mg/administration lesopitron, between 10 and 100 mg/administration gepirone, between 20 and 200 mg tandospirone, or between 10 and 100 mg zalospirone.

156. The method of any one of claims 150-155, wherein serotonin pre-synaptic autoreceptor antagonist is not administered during the first time period associated with each administration.

157. The method of any one of claims 150-156, wherein a serotonin pre-synaptic autoreceptor antagonist is administered during one or more of the second time periods.

158. The method of claim 149, wherein
the type of receptor is serotonin post-synaptic receptors;
the ligand is a serotonin post-synaptic autoreceptor antagonist; and
the undesirable mental or neurological condition is negatively linked to the serotonin post-synaptic autoreceptors.

159. The method of claim 158, wherein the counteradaptation is at least one of:
an increase in the biosynthesis and/or release of serotonin at the synaptic cleft;
a decrease in the reuptake of serotonin;
an increase in the number of serotonin post-synaptic receptors;
an increase in the sensitivity of the serotonin post-synaptic receptors to serotonin and/or serotonin post-synaptic receptor agonists;
a decrease in the number of serotonin pre-synaptic autoreceptors;
a decrease in the sensitivity of the serotonin pre-synaptic autoreceptors to serotonin and/or serotonin pre-synaptic autoreceptor agonists.

160. The method of any one of claims 158-159, wherein the serotonin post-synapatic receptors are 5HT₁ receptors; 5HT₂ receptors; 5HT₃ receptors; 5HT₄ receptors; 5HT₅ receptors; 5HT₆ receptors; 5HT₇ receptors; or receptors of a subtype thereof.

161. The method of any one of claims 158-160, wherein the serotonin post-synaptic receptor antagonist is (S)-WAY-100135, WAY-100635, buspirone, gepirone, ipsapirone, tandospirone, Lesopitron, zalospirone, MDL-73005EF, or BP-554.

162. The method of any one of claims 158-161, wherein the initial dosage of the serotonin post-synaptic receptor antagonist is equivalent to between about 0.01 and 5 mg/kg/administration of WAY-100635.

163. The method of any one of claims 158-161, wherein the initial dosage of the serotonin post-synaptic receptor antagonist is equivalent to between about 0.025 and 1 mg/kg/administration of WAY-100635.

164. The method of any one of claims 158-163, further comprising the step of
administering a serotonin pre-synaptic autoreceptor agonist in combination with the serotonin post-synaptic receptor antagonist.

165. The method of any one of claims 158-163, wherein the serotonin post-synaptic receptor antagonist is also a serotonin pre-synaptic autoreceptor agonist.

166. The method of any one of 158-164, wherein a serotonin post-synaptic receptor agonist is not administered during the first time period associated with each administration.

167. The method of any one of claims 158-161, wherein a serotonin post-synaptic receptor agonist is administered during one or more of the second time periods.

168. The method of any one of claims 158-167, further comprising the step of
administering a norepinephrine pre-synaptic alpha-2 adrenergic receptor agonist and/or a norepinephrine post-synaptic adrenergic receptor antagonist in combination with the ligand.

169. The method of any one of claims 149-168, wherein the up-regulation of the serotonin system causes a therapeutic benefit with respect to the undesirable mental or neurological condition.

170. The method of any one of claims 1-51, wherein
the neurotransmitter system is the norepinephrine system; and
the counteradaptation causes an up-regulation of the norepinephrine system.

171. The method of claim 170, wherein
the type of receptor is norepinephrine pre-synaptic alpha-2 adrenergic receptors;
the ligand is an norepinephrine pre-synaptic alpha-2 adrenergic receptor agonist; and
the undesirable mental or neurological condition is positively linked to the norepinephrine pre-synaptic alpha-2 adrenergic receptors.

172. The method of claim 171, wherein the counteradaptation is at least one of:
an increase in the biosynthesis and/or release of norepinephrine at the synaptic cleft;
a decrease in reuptake of norepinephrine;
a decrease in the number of norepinephrine pre-synaptic alpha-2 adrenergic receptors;
a decrease in the sensitivity of the norepinephrine pre-synaptic alpha-2 adrenergic receptors to norepinephrine and/or norepinephrine pre-synaptic alpha-2 adrenergic receptor agonists;
an increase in the number of norepinephrine post-synaptic adrenergic receptors; or
an increase in the sensitivity of the norepinephrine post-synaptic adrenergic receptors to norepinephrine and/or norepinephrine post-synaptic adrenergic receptor agonists.

173. The method of any one of claims 171-172, wherein the norepinephrine pre-synaptic alpha-2 adrenergic receptor agonist is clonidine, guanfacine, lofexidine, detomidine, dexmedetomidine, mivazerol, or alpha-methylnoradreniline.

174. The method of any one of claims 171-173, wherein the initial dosage of the norepinephrine pre-synaptic alpha-2 adrenergic receptor agonist is equivalent to between 0.1 and 10 μg/kg/administration of clonidine, between 0.01 and 10 mg/administration guanfacine, between 0.01 and 1 mg/administration lofexidine, between 1 and 100 μg/kg/administration detomidine, between 0.05 and 5 μg/kg/administration dexmedetomidine, between 0.05 and 10 μg/kg/administration mivazerol, or between 5 and 500 ng/kg/administration of alpha-methylnoradreniline.

175. The method of any one of claims 171-173, wherein the initial dosage of the norepinephrine pre-synaptic alpha-2 adrenergic receptor agonist is equivalent to between 0.1 and 0.5 mg/administration of clonidine, between 0.1 and 5 mg/administration guanfacine, between 0.05 and 0.5 mg/administration lofexidine, between 10 and 80 μg/kg/administration detomidine, between 0.1 and 3 μg/kg/administration dexmedetomidine, between 0.5 and 5 μg/kg/administration of mivazerol, or between 10 and 100 ng/kg/administration of alpha-methylnoradreniline.

176. The method of any one of claims 171-173, wherein norepinephrine pre-synaptic alpha-2 adrenergic receptor antagonist is not administered during the first time period associated with each administration.

177. The method of any one of claims 171-174, wherein a norepinephrine pre-synaptic alpha-2 adrenergic receptor antagonist is administered during one or more of the second time periods.

178. The method of claim 170, wherein
the type of receptor is norepinephrine post-synaptic adrenergic receptors;
the ligand is a norepinephrine post-synaptic adrenergic receptor antagonist; and
the undesirable mental or neurological condition is negatively linked to the norepinephrine post-synaptic adrenergic receptors.

179. The method of claim 178, wherein the counteradaptation is at least one of:
an increase in the biosynthesis or release of norepinephrine at the synaptic cleft;
a decrease in the reuptake of norepinephrine;
an increase in the number of norepinephrine post-synaptic adrenergic receptors;
an increase in the sensitivity of the norepinephrine post-synaptic adrenergic receptors to norepinephrine and/or norepinephrine post-synaptic adrenergic receptor agonists;
a decrease in the number of norepinephrine pre-synaptic alpha-2 adrenergic receptors; or
a decrease in the sensitivity of the norepinephrine pre-synaptic alpha-2 adrenergic receptors to norepinephrine and/or norepinephrine pre-synaptic alpha-2 adrenergic receptor agonists.

180. The method of any one of claims 178-179, wherein the norepinephrine post-synapatic adrenergic receptors are alpha receptors; beta receptors; or receptors of a subtype thereof.

181. The method of any one of claims 178-180, wherein the norepinephrine post-synaptic adrenergic receptor antagonist is idazoxan, SKF 104078, or SKF 104856.

182. The method of any one of claims 178-181, wherein the initial dosage of the norepinephrine post-synaptic adrenergic receptor antagonist is equivalent to between 0.5 and 100 mg/administration of idazoxan.

183. The method of any one of claims 178-181, wherein the initial dosage of the norepinephrine post-synaptic adrenergic receptor antagonist equivalent to between 5 and 50 mg/administration of idazoxan.

184. The method of any one of claims 178-183, further comprising the step of
administering a norepinephrine pre-synaptic alpha-2 adrenergic receptor agonist in combination with the norepinephrine post-synaptic adrenergic receptor antagonist.

185. The method of any one of claims 178-183, wherein the norepinephrine post-synaptic adrenergic receptor antagonist is also a norepinephrine pre-synaptic alpha-2 adrenergic receptor agonist.

186. The method of any one of claims 178-185, wherein a norepinephrine post-synaptic adrenergic receptor agonist is not administered during the first time period associated with each administration.

187. The method of any one of claims 178-186, wherein a norepinephrine post-synaptic adrenergic receptor agonist is administered during one or more of the second time periods.

188. The method of any one of claims 170-187, further comprising the step of
administering a serotonin pre-synaptic autoreceptor agonist or a serotonin post-synaptic receptor antagonist in combination with the ligand.

189. The method of any one of claims 149-188, wherein the condition is a mood disorder;
an eating disorder, a pain disorder, a substance abuse disorder, an anxiety disorder, or an obsessive-compulsive disorder.

190. The method of any one of claims 149-188, wherein the method is used to address the undesirable mental or neurological condition in a patient.

191. The method of any one of claims 149-190, wherein the method is used as an adjunct treatment for cancer.

192. The method of any one of claims 170-191, wherein the up-regulation of the norepinephrine system causes a therapeutic benefit with respect to the undesirable mental or neurological condition.

193. A method of inducing a regulation of a neurotransmitter system, the neurotransmitter system including a type of receptors linked to an undesirable mental or neurological condition, the method comprising the step of:
repeatedly administering to the patient a ligand for the type of receptor, each administration having an administration half-life, thereby causing the ligand to bind a substantial fraction of receptors of that type during a first time period associated with each administration, thereby inducing a counteradaptation,
wherein the counteradaptation causes the regulation of the neurotransmitter system during a second time period associated with each administration, the second time period being subsequent to the first time period.

194. The method of claim 193, wherein during each second time period, a substantial fraction of the receptors of the type of receptor remain unbound to the ligand.

195. The method according to claim 1-194 wherein one or more ligands selected from the group consisting substance P, endorphin, dynorphin, serotonin and norepinephrine receptor ligands may be administered simultaneously or sequentially.
